(19) United States
(12) Patent Application Publication
(10) Pub. No.: US 2024/0293339 A1
(43) Pub. Date: Sep. 5, 2024

(54) COMPOUNDS, COMPOSITIONS AND METHODS FOR THE PREVENTION AND/OR TREATMENT OF VARIOUS MITOCHONDRIAL DISEASES OR DISORDERS, INCLUDING FRIEDREICH'S ATAXIA

(71) Applicant: Stealth Bio Therapeutics Inc., Needham, MA (US)

(72) Inventors: Brian D. Gildea, Bedford, MA (US); Martin Redmon, Boston, MA (US); Dennis Keefe, Bedford, MA (US); Guozhu Zheng, Lexington, MA (US)

(73) Assignee: Stealth Bio Therapeutics Inc., Needham, MA (US)

(21) Appl. No.: 18/568,502

(22) PCT Filed: Jun. 9, 2022
(86) PCT No.: PCT/US22/32866

§ 371 Date: Dec. 8, 2023

Related U.S. Application Data
(60) Provisional application No. 63/209,159, filed on Jun. 10, 2021.
(60) Provisional application No. 63/313,149, filed on Feb. 23, 2022.

(51) Int. Cl. A61K 31/122; A61K 31/05; A61P 25/28; C07C 39/19; C07C 50/28

(52) U.S. Cl. CPC A61K 31/122; A61K 31/05; A61P 25/28; C07C 39/19; C07C 50/28; C07B 2200/05

(57) ABSTRACT

The disclosure provides various new and existing compounds for use alone or as formulated in a composition (e.g., medicaments) and related methods and uses for treating, preventing, inhibiting, ameliorating or delaying the onset of a disease, disorder or condition associated with ferroptosis in a mammalian subject. Such ferroptosis related diseases, disorders or conditions can include: Friedreich's ataxia, Leigh syndrome, Leber's Hereditary Optic Neuropathy (LHON), (proliferative, non-proliferative, diabetic or hypertensive) retinopathy, refractory epilepsy, Parkinson's disease (PD), Alzheimer's disease (AD), Huntington's disease (HD), Amyotrophic Lateral Sclerosis (ALS), ischemic stroke, a cardiomyopathy (e.g. cardiac ischemia-reperfusion injury, myocardial infarction, Barth cardiomyopathy, hypertrophic cardiomyopathy or heart failure), renal injury, renal ischemia reperfusion injury or acute renal failure.

### CROSS REFERENCE TO RELATED APPLICATIONS

This application claims the benefit of U.S. Provisional Patent Application Nos. 63/209,159, filed on Jun. 10, 2021, and 63/313,149, filed on Feb. 23, 2022, which are incorporated herein by reference in their entireties for any and all purposes.

### TECHNICAL FIELD

The present application relates generally to methods, medicaments, formulations and uses related to treating, preventing, inhibiting, ameliorating or delaying the onset of a disease, disorder or condition associated with ferroptosis in a mammalian subject suffering from said disease, disorder or condition comprising administering to the subject a therapeutically effective amount of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8), 2-((S,6E,10E)-3-hydroxy-3,7,11,15-tetramethylhexadeca-6,10,14-trien-1-yl)-3,5,6-trimethylbenzene-1,4-diol (Compound 1-2; No Known CAS#), (R,E)-2-(3-hydroxy-3,7,11-trimethyldodeca-6,10-dien-1-yl)-3,5,6-trimethylcyclohexa-2,5-diene-1,4-dione (Compound 2-1; No Known CAS#), (R,E)-2-(3-hydroxy-3,7,11-trimethyldodeca-6,10-dien-1-yl)-3,5,6-trimethylbenzene-1,4-diol (Compound 2-2; No Known CAS#), (S,E)-2-(3-hydroxy-3,7,11-trimethyldodeca-6,10-dien-1-yl)-3,5,6-trimethylcyclohexa-2,5-diene-1,4-dione (Compound 3-1; No Known CAS#), (S,E)-2-(3-hydroxy-3,7,11-trimethyldodeca-6,10-dien-1-yl)-3,5,6-trimethylbenzene-1,4-diol (Compound 3-2; No Known CAS#), (R)-2-(3-hydroxy-3,7-dimethyloct-6-en-1-yl)-3,5,6-trimethylcyclohexa-2,5-diene-1,4-dione (Compound 4-1; No Known CAS#), (R)-2-(3-hydroxy-3,7-dimethyloct-6-en-1-yl)-3,5,6-trimethylbenzene-1,4-diol (Compound 4-2; CAS# 116119-16-5), (S)-2-(3-hydroxy-3,7-dimethyloct-6-en-1-yl)-3,5,6-trimethylcyclohexa-2,5-diene-1,4-dione (Compound 5-1; No Known CAS#), (S)-2-(3-hydroxy-3,7-dimethyloct-6-en-1-yl)-3,5,6-trimethylbenzene-1,4-diol (Compound 5-2; No Known CAS#) or a pharmaceutically acceptable salt, hydrate, solvate, and/or tautomer of any of the foregoing. Said disease, disorder or condition involving ferroptosis may include, but not necessarily be limited to, Friedreich's ataxia, Leigh syndrome, Leber's Hereditary Optic Neuropathy (LHON), (proliferative, non-proliferative, diabetic or hypertensive) retinopathy, refractory epilepsy, Parkinson's disease (PD), Alzheimer's disease (AD), Huntington's disease (HD), Amyotrophic Lateral Sclerosis (ALS), ischemic stroke, a cardiomyopathy (e.g. cardiac ischemia-reperfusion injury, myocardial infarction, Barth cardiomyopathy, hypertrophic cardiomyopathy or heart failure), renal injury, renal ischemia reperfusion injury or acute renal failure.

### INTRODUCTION

The following description is provided to assist the understanding of the reader. None of the information provided or references cited is admitted as being prior art to the compositions and methods disclosed herein.

Friedreich's ataxia (FA) is a fatal, monogenic, autosomal recessive disease caused by mutations in the gene encoding the nuclear encoded mitochondrial protein frataxin. Tissues in both the peripheral and central nervous systems are affected in FA, and include the dentate nucleus, Clark's column, spinocerebellar tract and dorsal root ganglia. Progressive degeneration of these tissues leads to a worsening ataxia which for most patients ends in loss of independent ambulation by the third decade of life.

Symptoms of Friedreich's ataxia typically begin between the ages of 5 and 15 years, although they sometimes appear in adulthood. The first symptom to appear is usually gait ataxia, or difficulty walking. The ataxia gradually worsens and slowly spreads to the arms and the trunk. There is often loss of sensation in the extremities, which may spread to other parts of the body. Other features include loss of tendon reflexes, especially in the knees and ankles. Most people with Friedreich's ataxia develop scoliosis, which often requires surgical intervention for treatment. Dysarthria (slowness and slurring of speech) develops and can get progressively worse. Many individuals with later stages of Friedreich's ataxia develop hearing and vision loss.

Heart disease often accompanies Friedreich's ataxia, such as hypertrophic cardiomyopathy, myocardial fibrosis (formation of fiber-like material in the muscles of the heart), and cardiac (heart) failure. Heart rhythm abnormalities such as tachycardia (fast heart rate) and heart block (impaired conduction of cardiac impulses within the heart) are also common. Other symptoms that may occur include chest pain, shortness of breath, and heart palpitations.

There is no known cure for Friedreich's ataxia. Generally, therapies involve treatment of the symptoms. Because patients with Friedreich's ataxia are at a risk of developing heart disease, they are often prescribed medications such as beta blockers, ACE inhibitors and/or diuretics. Because it is believed that damage caused by oxidative stress is involved in the progression of Friedreich's ataxia, antioxidants such as vitamin E, idebenone and coenzyme Q10 are often co-administered to persons diagnosed or suspected of having Friedrich's ataxia. These compounds have been used in various clinical trials.

Ferroptosis is an iron-dependent type of cell death that is biochemically distinct from apoptosis and typically accompanied by a large amount of iron accumulation and lipid peroxidation during the cell death process. Ferroptosis-inducing factors can directly or indirectly affect glutathione peroxidase through different pathways, resulting in a decrease in antioxidant capacity and accumulation of lipid reactive oxygen species (ROS) in cells, ultimately leading to oxidative cell death. Recent studies have shown that ferroptosis is closely related to the pathophysiological processes of many diseases, such as tumors, nervous system diseases, ischemia-reperfusion injury, kidney injury, and blood diseases. Decreased expression of frataxin (FXN) is associated with mitochondrial dysfunction, mitochondrial iron accumulation, and increased oxidative stress. Recent studies have shown that frataxin, which modulates iron homeostasis and mitochondrial function, is a key regulator of ferroptosis. As such, ferroptosis as has been identified as a therapeutic target for treating Friedreich's ataxia and other diseases/disorders/conditions where ferroptosis appears to be associated with disease/disorder/condition progression. As described above, ferroptosis is associated with glutathione depletion and production of lipid peroxides, which are generated by lipoxygenase enzymes such as lipoxygenase-15. Accordingly, targeting lipoxygenase-15 (and other lipoxygenases) provides a further therapeutic target for Friedreich's ataxia and other diseases/disorders/conditions where excess lipid oxidation appears to be associated with disease/disorder/condition progression.

Currently, EPI-743 (a benzoquinone compound also known as vatiquinone—which compound is 2-[(3R,6E, 10E)-3-Hydroxy-3,7,11,15-tetramethyl-6,10,14-hexadecatrien-1-yl]-3,5,6-trimethyl-2,5-cyclohexadiene-1,4-dione; CAS# 1213269-98-7) is currently recruiting a phase 2/3 clinical trial for the treatment of Friedreich's ataxia. Vatiquinone (EPI-743) has been evaluated in clinical trials for the treatment of various other diseases or disorders, including, refractory epilepsy, Leigh syndrome, respiratory chain diseases, noise-induced hearing loss, Pearson syndrome, Leber's Hereditary Optic Neuropathy (LHON), Autism Spectrum disorder, Tourette Syndrome, Rett Syndrome, Parkinson's disease and other disorders. Vatiquinone is believed to reduce oxidative stress and improve mitochondrial function. It does not appear that the S-enantiomer of vatiquinone (i.e. 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8)), or any of the other compounds listed above as Compounds 1-2, 2-1, 2-2, 3-1, 3-2, 4-1, 4-2, 5-1 or 5-2 have ever been evaluated in the clinic for the treatment of any of these diseases/disorders/conditions.

Several other therapies for the treatment of Friedreich's ataxia are currently in clinical trials but there are no FDA approved drugs. Ferroptosis is a newly described phenomenon and there are no approved drugs that have been developed to address this phenomenon/mode of action as it relates to an indication or medical condition. Hence, there remains a need for better drug candidates to address the needs of patients diagnosed with Friedreich's ataxia and other diseases/disorders/conditions in which ferroptosis and/or lipid peroxidation plays a role in the pathology.

### BRIEF DESCRIPTION OF THE DRAWINGS

FIG. 1 is an illustration of a chemical structure of vatiquinone (CAS# 1213269-98-7), its S-enantiomer 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8)) and 2-((S,6E,10E)-3-hydroxy-3,7,11,15-tetramethylhexadeca-6,10,14-trien-1-yl)-3,5,6-trimethylbenzene-1,4-diol (Compound 1-2; No Known CAS#), the reduced form of Compound 1-1.

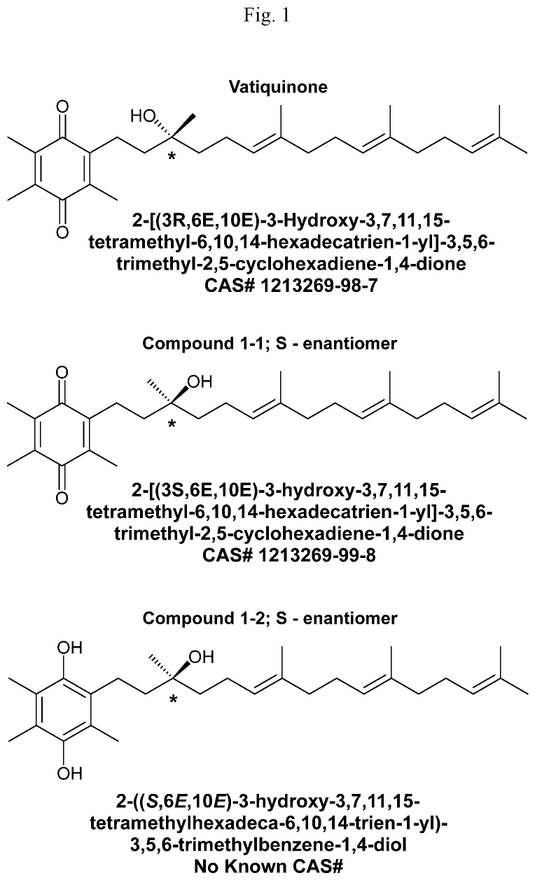

FIG. 2 is an illustration of the chemical structure of (R,E)-2-(3-hydroxy-3,7,11-trimethyldodeca-6,10-dien-1-yl)-3,5,6-trimethylcyclohexa-2,5-diene-1,4-dione (Compound 2-1; No Known CAS#) and (R,E)-2-(3-hydroxy-3,7,11-trimethyldodeca-6,10-dien-1-yl)-3,5,6-trimethylbenzene-1,4-diol (Compound 2-2; No Known CAS#), the reduced form of Compound 2-1.

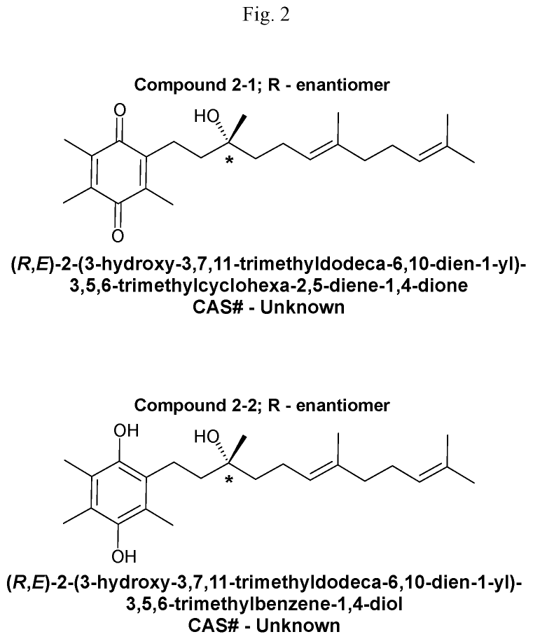

FIG. 3 is an illustration of the chemical structure of (S,E)-2-(3-hydroxy-3,7,11-trimethyldodeca-6,10-dien-1-yl)-3,5,6-trimethylcyclohexa-2,5-diene-1,4-dione (Compound 3-1; No Known CAS#) and (S,E)-2-(3-hydroxy-3,7,11-trimethyldodeca-6,10-dien-1-yl)-3,5,6-trimethylbenzene-1,4-diol (Compound 3-2; No Known CAS#), the reduced form of Compound 3-1.

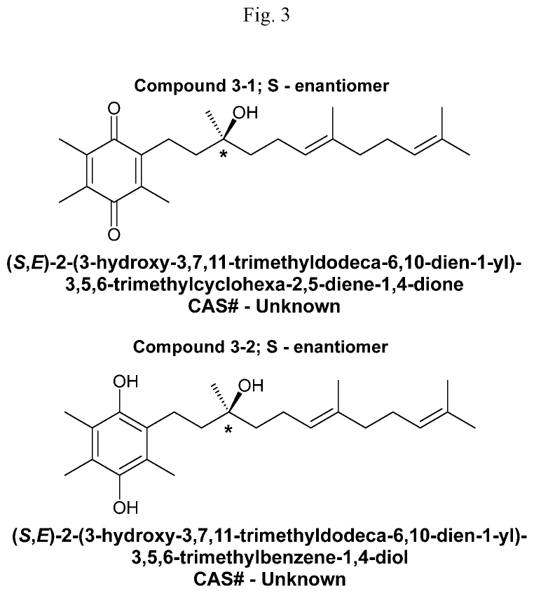

FIG. 4 is an illustration of the chemical structure of (R)-2-(3-hydroxy-3,7-dimethyloct-6-en-1-yl)-3,5,6-trimethylcyclohexa-2,5-diene-1,4-dione (Compound 4-1; No Known CAS#) and (R)-2-(3-hydroxy-3,7-dimethyloct-6-en-1-yl)-3,5,6-trimethylbenzene-1,4-diol (Compound 4-2; CAS# 116119-16-5), the reduced form of Compound 4-1.

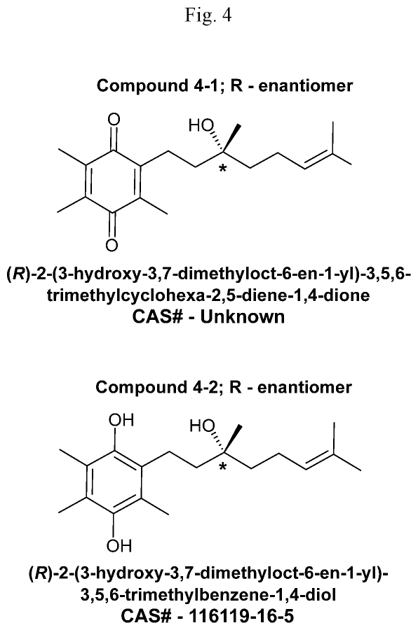

FIG. 5 is an illustration of the chemical structure of (S)-2-(3-hydroxy-3,7-dimethyloct-6-en-1-yl)-3,5,6-trimethylcyclohexa-2,5-diene-1,4-dione (Compound 5-1; No Known CAS#) and (S)-2-(3-hydroxy-3,7-dimethyloct-6-en-1-yl)-3,5,6-trimethylbenzene-1,4-diol (Compound 5-2; No Known CAS#), the reduced form of Compound 5-1.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

### I. Chemical Definitions

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

The abbreviations used herein have their conventional meaning within the chemical and biological arts. The chemical structures and formulae set forth herein are intended to comply with the standard rules of chemical valency known in the chemical arts. When a range of values is listed, it is intended to encompass each value and subrange within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₅, C₃-C₄, C₄-C₆, C₄-C₅, and C₅-C₆ alkyl.

Certain compounds of the present application can exist in unsolvated forms as well as solvated forms, including hydrated forms. Solvated forms can exist, for example, because it is difficult or impossible to remove all the solvent from the compound post synthesis. In general, the solvated forms are equivalent to unsolvated forms and are encompassed within the scope of the present application. Certain compounds of the present application may exist in multiple crystalline or amorphous forms. Certain compounds of the present application may exist in various tautomeric forms. Certain compounds of the present application may exist in various salt forms. In general, all physical forms are equivalent for the uses contemplated by the present application and are intended to be within the scope of the present disclosure.

As used herein, the term “hydrate” refers to a compound which is associated with water. The number of the water molecules contained in a hydrate of a compound may be (or may not be) in a definite ratio to the number of the compound molecules in the hydrate.

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, the term “solvate” refers to forms of the compound that are associated with a solvent, possibly by a solvolysis reaction. This physical association may include hydrogen bonding. Conventional solvents include water, methanol, ethanol, isopropanol, acetic acid, ethyl acetate, acetone, hexane(s), DMSO, THF, diethyl ether, and the like.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

### II. Other Definitions

It is to be appreciated that certain aspects, modes, embodiments, variations and features of the technology are described below in various levels of detail in order to provide a substantial understanding of the present application. The definitions of certain terms as used in this specification are provided below. Unless defined otherwise, all technical and scientific terms used herein generally have the same meaning as commonly understood by one of ordinary skill in the art to which this technology belongs.

As used in this specification and the appended embodiments and/or claims, the singular forms “a”, “an” and “the” include plural references unless the content clearly dictates otherwise. For example, reference to “a cell” includes a combination of two or more cells, and the like.

As used herein, the “administering” or the “administration” of an agent (i.e. therapeutic agent) or compound/drug product/composition to a subject (which subject may be in need thereof) includes any route of introducing or delivering to a subject a compound/drug product to perform its intended function. Administration may be carried out by any suitable route, such as oral administration. Administration can be carried out subcutaneously. Administration can be carried out intravenously. Administration can be carried out intraocularly. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or administration by use of a device (e.g. an infusion pump).

As used herein, to “ameliorate” a disease, disorder or condition associated with ferroptosis (e.g., a disease such as Friedreich's ataxia where ferroptosis is a known to occur in subjects suffering from Friedreich's ataxia) refers to results that, in a statistical sample or specific subject, make the occurrence of the disorder, disorder or condition (or a sign, symptom or condition thereof) better or more tolerable in a sample or subject administered a therapeutic agent relative to a control sample or subject.

As used herein the terms “carrier” and “pharmaceutically acceptable carrier” refer to a diluent, adjuvant, excipient, or vehicle with which a compound/drug product/composition (including a medicament) is administered or formulated for administration. Non-limiting examples of such pharmaceutically acceptable carriers include liquids, such as water, saline, oils, and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E.W. Martin, herein incorporated by reference in its entirety.

As used herein, the phrase “delaying the onset of” refers to, in a statistical sample, postponing, hindering, or causing one or more signs, symptoms or conditions of a disease, disorder or condition associated with ferroptosis to occur more slowly than normal in a treated sample (or subject) relative to an untreated control sample (or subject).

As used herein, the term “effective amount” refers to a quantity of a compound/drug product/composition sufficient to achieve a desired therapeutic and/or prophylactic effect, e.g., an amount that treats, prevents, inhibits, ameliorates, or delays the onset of the disease, disorder or condition associated with ferroptosis or the physiological signs, symptoms or conditions of the disease or disorder. In the context of therapeutic or prophylactic applications, in some embodiments, the amount of a compound/composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/drug products/compositions can also be administered in combination with one or more additional therapeutic compounds (a so called “co-administration” where, for example, the additional therapeutic compounds/drug products/compositions could be administered simultaneously, sequentially or by separate administration). The one or more additional therapeutic compounds could be, for example, a beta blocker, ACE inhibitor and/or diuretic used to treat a patient experiencing at risk of heart disease or heart failure. The one or more additional therapeutic compounds could be, for example, a Szeto-Schiller peptide such as SS-20 or SS-31 (a.k.a. elamipretide or bendavia). The one or more additional therapeutic compounds could be, for example, a peptidomimetic such as those described and claimed in WIPO published application: WO2019/118878.

In the methods described herein, one or more of Compounds 1-1, 1-1, 2-1, 2-2, 3-1, 3-2, 4-1, 4-2, 5-1 and 5-2, or pharmaceutically acceptable salts, tautomers, hydrates, and/or solvates thereof, may be administered to a subject having one or more signs, symptoms, or conditions of a disease or disorder associated with ferroptosis, such as Friedreich's ataxia; e.g., muscle weakness, especially in the arms and legs, loss of coordination, motor control impairment, vision impairment, hearing impairment, slurred speech, curvature of the spine, diabetes, heart and/or ophthalmic conditions or disorders. For example, a “therapeutically effective amount” of the one or more of Compounds 1-1, 1-1, 2-1, 2-2, 3-1, 3-2, 4-1, 4-2, 5-1 and 5-2 so administered may create in-vivo levels at which the presence, frequency, or severity of one or more signs, symptoms, or conditions (e.g. risk factors) of a disease, disorder or condition associated with ferroptosis, is treated, prevented, inhibited, ameliorated or delayed. In some embodiments, administration of a therapeutically effective amount of one or more of Compounds 1-1, 1-1, 2-1, 2-2, 3-1, 3-2, 4-1, 4-2, 5-1 and 5-2 so administered, treats, inhibits, ameliorates, prevents or delays the onset of in a subject, the physiological effects of a disease, disorder or condition associated with ferroptosis.

As used herein, “inhibit” or “inhibiting” refers to the reduction in a sign, symptom or condition (e.g. risk factor) associated with a disease, disorder or condition associated with ferroptosis by an objectively measurable amount or degree compared to a control. In one embodiment, inhibit or inhibiting refers to the reduction by at least a statistically significant amount compared to a control or control subject. In one embodiment, inhibit or inhibiting refers to a reduction by at least 5 percent compared to control or control subject. In various individual embodiments, inhibit or inhibiting refers to a reduction by at least 2, 3, 4, 5, 10, 15, 20, 25, 30, 33, 40, 50, 60, 67, 70, 75, 80, 90, 95, or 99 percent compared to a control or control subject.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

As used herein, the terms “treating” or “treatment” refer to therapeutic treatment, wherein the object is to reduce, alleviate or slow down (lessen) a pre-existing disease, disorder or condition associated with ferroptosis. By way of example, but not by way of limitation, a subject is successfully “treated” for a disease (e.g. Friedreich's ataxia) if, after receiving an effective amount of the compound/drug product/composition or a pharmaceutically acceptable salt, enantiomer, tautomer, hydrate, and/or solvates thereof, according to the methods described herein, the subject shows observable and/or measurable reduction in or absence of one or more signs, symptoms or conditions associated with the disease or disorder (e.g. Friedreich's ataxia). For example, a subject afflicted with Friedreich's ataxia may experience observable and/or measurable reduction in or absence of muscle weakness, especially in the arms and legs, loss of coordination, motor control impairment, vision impairment, hearing impairment, slurred speech, curvature of the spine, diabetes, heart and/or ophthalmic conditions. It is also to be appreciated that the various modes of treatment of medical conditions as described are intended to mean “substantial,” which includes total alleviation of conditions, signs or symptoms of the disease, disorder or condition as well as “partial” where some biologically or medically relevant result is achieved.

As used herein, “prevention” or “preventing” of a disease, disorder or condition associated with ferroptosis refers to results that, in a statistical sample, exhibit a reduction in the occurrence of the disorder, disorder or condition in the a sample or subject administered the therapeutic agent relative to a control sample or subject, or exhibit a delay in the onset of one or more symptoms of the disorder, disorder or condition relative to a control sample or subject. Such prevention is sometimes referred to as a prophylactic treatment.

### III. Chiral/Stereochemistry Considerations

Compounds described herein can comprise one or more asymmetric centers, and thus can exist in various isomeric forms, e.g., enantiomers and/or diastereomers (i.e., stereoisomers). Chiral centers in illustrated structures (including the embodiements and/or claims) may be identified herein by use of an asterisk (*). Except as otherwise stated, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The disclosure of the present application additionally may encompass compounds described herein as individual isomers/enantiomers substantially free of other isomers/enantiomers, and alternatively, as mixtures of various isomers/enantiomers. However, when specific chirality is illustrated, it is intended to denote that the pure compound of that specific chirality is intended as compared with a mixture of enantiomers/diastereoisomers.

As used herein, a pure enantiomeric compound is substantially free from other enantiomers or stereoisomers of the compound (i.e., in enantiomeric excess); as purity is a relative term in the sense that it is exceedingly difficult to achieve 100% purity. In other words, an “S” form of the compound is substantially free from the “R” form of the compound and is, thus, in enantiomeric excess of the “R” form. With respect to amino acids (which are more commonly described in terms of “D” and “L” enantiomer, it is to be understood that in most cases for a “D”-amino acid the configuration is “R” and for an “L”-amino acid, the configuration is “S” (cystine being a known exception). In some embodiments, ‘substantially free’, refers to: (i) an aliquot of an “S” form compound that contains less than 1-2% “R” form; or (ii) an aliquot of an “R” form compound that contains less than 1-2% “S” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the particularly identified enantiomer (e.g. as compared with the other enantiomer). In certain embodiments, the relative weights are based only upon the R and S forms with respect to a certain stereocenter of the compound of interest. In certain embodiments, the relative weights are based upon total weight of all enantiomers, diastereoisomer or other stereoisomers of the compound.

In the compositions provided herein, an enantiomerically pure compound can be present with other active or inactive ingredients. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

### IV. Pharmaceutical Compositions, Routes of Administration, and Dosing

In some embodiments, the present application is directed to a pharmaceutical composition. In some embodiments, the composition comprises a compound of the present application and a pharmaceutically acceptable carrier. In certain embodiments, the pharmaceutical composition comprises a plurality of compounds of the present application and a pharmaceutically acceptable carrier. The pharmaceutical composition can be a medicament.

In certain embodiments, a pharmaceutical composition may further comprise at least one additional therapeutic agent other than a compound or compounds disclosed herein (e.g. Compounds 1-1, 1-2, 2-1, 2-2, 3-1, 3-2, 4-1, 4-2, 5-1 and/or 5-2). The at least one additional therapeutic agent can be an agent useful in the treatment of a disease associated with ferroptosis, such as Friedreich's ataxia. Thus, in some embodiments, pharmaceutical compositions of the present application can be prepared, for example, by combining one or more compounds with a pharmaceutically acceptable carrier and, optionally, one or more additional therapeutical agents.

Pharmaceutical compositions of the present application may contain an effective amount of a therapeutic compound (or compounds) as described herein and may optionally be disbursed (e.g. dissolved, suspended or otherwise) in a pharmaceutically acceptable carrier. The components of the pharmaceutical composition(s) may also be capable of being commingled with the compounds of the present application, and with each other, in a manner such that there is no interaction that would substantially impair the desired pharmaceutical efficiency.

As stated above, an “effective amount” refers to any amount of the active compound (or compounds; alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e. preventative) or therapeutic treatment regimen can be planned which does not cause substantial unwanted toxicity and yet is effective to treat the particular condition or disease of a particular subject. The effective amount for any particular indication can vary depending on such factors as the disease, disorder or condition being treated, the particular compound or compounds being administered, the size of the subject, and/or the severity of the disease, disorder or condition. The effective amount may be determined during pre-clinical trials and/or clinical trials by methods familiar to physicians and clinicians. One of ordinary skill in the art can empirically determine the effective amount of a particular compound and/or other therapeutic agent(s) without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g. a pump).

For any compound described herein the therapeutically effective amount can, for example, be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Compounds (alone or as formulated in a pharmaceutical composition) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing, any of the animal model system known in the art can be used prior to administration to human subjects. In some embodiments, dosing can be tested directly in humans.

Dosage, toxicity and therapeutic efficacy of any therapeutic compounds/agents, compositions (e.g. drug product, formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, in such cases it may be prudent to design a delivery system that targets such compounds to the site of affected tissue in order to minimize potential damage to uninfected cells and, thereby, reduce side effects.

In some embodiments, an effective amount of a therapeutic compound/agent disclosed herein sufficient for achieving a therapeutic or prophylactic effect, can range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 100 mg/kg body weight every day, every two days or every three days or within the range of 1-100 mg/kg every week, every two weeks or every three weeks. In some embodiments, a single dosage of a therapeutic compound/agent disclosed herein ranges from 0.001-10,000 micrograms per kg body weight. In some embodiments, a therapeutic compound/agent disclosed herein dissolved or suspended in a carrier range from 0.2 to 2000 micrograms per delivered milliliter. In some embodiments, the dose regimen meets pharmacokinetic target concentrations in target tissues to achieve a desired therapeutic outcome.

An exemplary treatment regime can entail administration once per day, twice per day, thrice per day, once a week or once a month. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of a therapeutic compound/agent disclosed herein may be defined as a concentration of compound existing at the target tissue of 10⁻¹² to 10⁻⁴ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., oral, systemic, topical, subcutaneous, intra-nasal, parenteral infusion or transdermal application).

In some embodiments, intravenous or subcutaneous administration of a compound (alone or as formulated) may typically be from 0.01 μg/kg/day to 80 mg/kg/day. In some embodiments, intravenous or subcutaneous administration of a compound (alone or as formulated) may typically be from 0.01 μg/kg/day to 100 μg/kg/day. In some embodiments, intravenous or subcutaneous administration of a compound (alone or as formulated) may typically be from 0.1 μg/kg/day to 10 mg/kg/day. In some embodiments, intravenous or subcutaneous administration of a compound (alone or as formulated) may typically be from 10 μg/kg/day to 2 mg/kg/day. In some embodiments, intravenous or subcutaneous administration of a compound (alone or as formulated) may typically be from 500 μg/kg/day to 5 mg/kg/day. In some embodiments, intravenous or subcutaneous administration of a compound (alone or as formulated) may typically be from 1 mg/kg/day to 100 mg/kg/day. In some embodiments, intravenous or subcutaneous administration of a compound (alone or as formulated) may typically be from 1 mg/kg/day to 50 mg/kg/day.

Generally, daily oral doses of a compound (alone or as formulated) will be, for human subjects, from about 0.01 micrograms/kg per day to 250 milligrams/kg per day. In some embodiments, daily oral doses of a compound (alone or as formulated) will be, for human subjects, from about 1 milligrams/kg per day to 100 milligrams/kg per day or from about 10 milligrams/kg per day to 75 milligrams/kg per day or It is expected that oral doses of a compound (alone or as formulated) in the range of 0.1 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For use in therapy, an effective amount of the compound (alone or as formulated) can be administered to a subject by any mode that delivers the compound to the desired surface. Administering a pharmaceutical composition may be accomplished by any means known to the skilled artisan. Routes of administration include but are not limited to oral, topical, intranasal, systemic, intravenous, subcutaneous, intraperitoneal, intradermal, intraocular, ophthalmical, intrathecal, intracerebroventricular, iontophoretical, transmucosal, intravitreal, or intramuscular administration. Administration includes self-administration, the administration by another and administration by a device.

A therapeutic compound/agent disclosed herein can be delivered to the subject in a formulation or medicament (i.e. a pharmaceutical composition). Formulations and medicaments can be prepared by, for example, dissolving or suspending a therapeutic compound/agent disclosed herein in water, solvent, salt solution (e.g. NaCl or sodium phosphate), buffering agents, preservatives, compatible carriers, adjuvants, excipients, vehicles, a pharmaceutically acceptable carrier and optionally other therapeutically acceptable ingredients.

The pharmaceutical compositions (e.g. a formulation or medicament) can include a carrier (e.g. a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars (e.g. trehalose), polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g. a formulation or medicament) used for parenteral, intradermal, subcutaneous or intraocular application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 1, 2, 3, 4, 5, 6, 7 days or more of treatment).

The therapeutic compounds/agents or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound/agent can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g. a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g. a formulation or medicament) can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For oral administration, the compounds can be formulated readily by combining the active compound(s) with pharmaceutically acceptable carriers well known in the art. Such carriers enable the compounds of the present application to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above that may be chemically modified so that oral delivery of the derivative is efficacious. Generally, the chemical modification contemplated is the attachment of at least one moiety to the therapeutic agent(s), ingredient(s), and/or excipient(s), where said moiety permits (a) inhibition of acid hydrolysis; and (b) uptake into the blood stream from the stomach or intestine. Also desired is the increase in overall stability of the therapeutic agent(s), ingredient(s), and/or excipient(s)and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of the compound of the present application (or derivative) or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound/agent or pharmaceutical composition can be included in the formulation as fine multi-particulates in the form of granules or pellets of particle size about 1-2 mm. The formulation of the material for capsule administration could also be as a powder, lightly compressed plugs or even as tablets. The therapeutic compound/agent or pharmaceutical composition could be prepared by compression.

Colorants and flavoring agents may all be included. For example, the compound or pharmaceutical composition of the present application (or derivative) may be formulated and then further contained within an edible product, such as a refrigerated beverage containing colorants and flavoring agents.

One may dilute or increase the volume of the therapeutic compound/agent or pharmaceutical composition with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound or composition into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic compound/agent or composition (e.g. a medicament) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the present application or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Solutions, gels, ointments, creams or suspensions may be administered topically. The compounds may also be formulated in rectal or vaginal compositions such as suppositories or retention enemas, e.g., containing conventional suppository bases such as cocoa butter or other glycerides.

For administration by inhalation, compounds or compositions (e.g. medicament) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament or therapeutic compound/agent can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound/agent and a suitable powder base such as lactose or starch. Alternatively, the active compounds may be in powder form for constitution with a suitable vehicle, e.g., sterile pyrogen-free water, before use.

Nasal delivery of a therapeutic compound/agent or pharmaceutical composition of the present application is also contemplated. Nasal delivery allows the passage of a therapeutic compound/agent or pharmaceutical composition to the blood stream directly after administering the therapeutic compound/agent or pharmaceutical composition to the nose, without the necessity for deposition of the product in the lung. Formulations for nasal delivery include those with dextran or cyclodextran.

For nasal administration, a useful device is a small, hard bottle to which a metered dose sprayer is attached. In some embodiments, the metered dose is delivered by drawing the pharmaceutical composition of the present application solution into a chamber of defined volume, which chamber has an aperture dimensioned to aerosolize and aerosol formulation by forming a spray when a liquid in the chamber is compressed. The chamber is compressed to administer the therapeutic compound/agent or pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound/agent or pharmaceutical composition.

Also contemplated herein is pulmonary delivery of the compounds/agents disclosed herein. The compound/agent or pharmaceutical composition is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5): 143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this technology are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the compound(s)/therapeutic agent(s). Typically, each formulation is specific to the type of device employed and may involve the use of an appropriate propellant material, in addition to the usual diluents, adjuvants and/or carriers useful in therapy. Also, the use of liposomes, microcapsules, microspheres, nanoparticles, nanospheres, inclusion complexes, or other types of carriers is contemplated. Chemically modified compound of the present application may also be prepared in different formulations depending on the type of chemical modification or the type of device employed.

Formulations suitable for use with a nebulizer, either jet or ultrasonic, can, for example, comprise a compound/therapeutic agent (or derivative) dissolved in water at a concentration of about 0.01 to 50 mg of biologically active compound per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the present application caused by atomization of the solution in forming the aerosol.

Formulations for use with a metered-dose inhaler device may generally comprise a finely divided powder containing the compound/agent (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing compound of the present application (or derivative) and may also include a bulking agent, such as lactose, sorbitol, sucrose, or mannitol in amounts which facilitate dispersal of the powder from the device, e.g., 50 to 90% by weight of the formulation. The compound(s)/agent(s) (or derivative(s) thereof) can advantageously be prepared in particulate or nanoparticulate form with an average particle size of less than 10 micrometers (μm), most preferably 0.5 to 5 μm, for most effective delivery to the deep lung.

For ophthalmic or intraocular indications, any suitable mode of delivering the therapeutic compound(s)/agent(s) or pharmaceutical compositions (e.g. medicament(s)) to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C.V. Mosby Co., St. Louis (1983). Nonlimiting examples of pharmaceutical compositions suitable for administration in or near the eye include, but are not limited to, ocular inserts, minitablets, and topical formulations such as eye drops, ointments, and in situ gels. In one embodiment, a contact lens is coated with a pharmaceutical composition comprising a therapeutic compound/agent disclosed herein. In some embodiments, a single dose can comprise from between 0.1 ng to 5000 μg, 1 ng to 500 μg, or 10 ng to 100 μg of the therapeutic compound(s)/agent(s) or pharmaceutical compositions administered to the eye.

Eye drops can comprise a sterile liquid formulation that can be administered directly to the eye. In some embodiments, eye drops comprise at least one therapeutic compound/agent disclosed herein and may further comprise one or more preservatives. In some embodiments, the optimum pH for eye drops equals that of tear fluid and is about 7.4. For eye drops, the therapeutic compound/agent can be present in the drop solution from about 0.1% to about 5% (w/v or v/v depending on the physical nature (i.e. solid or liquid) of the active ingredient). In some embodiments, the therapeutic compound/agent can be present in the drop solution from about 1% to about 3% (w/v or v/v, as appropriate).

In situ gels are viscous liquids, showing the ability to undergo sol-to-gel transitions when influenced by external factors, such as appropriate pH, temperature, and the presence of electrolytes. This property causes slowing of drug drainage from the eyeball surface and increase of the active ingredient bioavailability. Polymers commonly used in in situ gel formulations include, but are not limited to, gellan gum, poloxamer, silicone containing formulations, silica-based formulations and cellulose acetate phthalate. In some embodiments, the therapeutic compound/agent is formulated into an in-situ gel (as the pharmaceutical composition/medicament).

For topical ophthalmic administration, therapeutic compound/agent or pharmaceutical composition may be formulated as solutions, gels, ointments, creams, suspensions, etc. as are well-known in the art. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and are more stable than conventional dosage forms. They also offer advantages such as accurate dosing of one or more therapeutic compounds, slow release of one or more therapeutic compounds/agents with constant speed and limiting of one or more therapeutic compounds'/agents' systemic absorption. In some embodiments, an ocular insert comprises one or more therapeutic compounds as disclosed herein and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers. An ocular insert can comprise silica. An ocular insert can comprise liposomes, nanoparticles or microparticles of degradable or biodegradable polymer (as described in more detail below).

Minitablets are biodegradable, solid drug forms, that transit into gels after application to the conjunctival sac, thereby extending the period of contact between active ingredient (i.e. the therapeutic compound(s)/agent(s) disclosed herein) and the eyeball surface, which in turn increases a therapeutic compounds'/agents' bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets can comprise one or more of the therapeutic compounds disclosed herein and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol® or carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular formulations and medicaments may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

In some embodiments, the viscosity of the ocular formulation comprising one or more therapeutic compounds/agents is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

In addition to the formulations described above, a therapeutic compound/agent disclosed herein may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some embodiments, the therapeutic compound(s)/agent(s) is/are administered as a depot formulation wherein the active therapeutic agent(s) is/are encapsulated by, or disposed within, silica-based microparticles. In some embodiments, the ocular formulation can be injected into the eye, for example as a sol-gel (e.g. a silica sol-gel). In some embodiments, the ocular formulation is a depot formulation such as a controlled release formulation (see below). Such controlled release formulation may comprise particles, such as microparticles or nanoparticles.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, silica/silicone and polymers such as polyethylene glycols.

Suitable liquid or solid pharmaceutical preparation forms can, for example, be aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions can be suitable for use in a variety of drug delivery systems. For a brief review of methods for drug delivery, see Langer R, Science 249:1527-33 (1990).

The therapeutic agent(s), including specifically but not limited to a therapeutic compound/agent disclosed herein, may be provided in particles. Particles as used herein means nanoparticles or microparticles (or in some instances larger particles) which can consist in whole or in part of the therapeutic compound or the other therapeutic agent(s) as described herein. The particles may contain the therapeutic compound(s)/agent(s) in a core surrounded by a coating, including, but not limited to, an enteric coating. The therapeutic compound(s)/agent(s) also may be dispersed throughout the particles. The therapeutic compound(s)/agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic compound(s)/agent(s), any of those materials routinely used in the art of pharmacy and medicine, including, but not limited to, erodible, non-erodible, biodegradable, or nonbiodegradable material or combinations thereof. The particles may be microcapsules which contain the compound(s)/agent(s) in a solution or in a semi-solid state. The particles may be of virtually any shape.

Both non-biodegradable and biodegradable polymeric materials can be used in the manufacture of particles for delivering the therapeutic compound(s)/agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, polyethylene glycols (PEGs), polyvinylalcohols (PVAs), poly(methyl methacrylates), poly(ethyl methacrylates), poly(butylmethacrylate), poly(isobutyl methacrylate), poly-lactic acid (PLA), poly(lactic-co-glycolic) acid (PLGA), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and poly(&-caprolactone) or mixtures of two or more of the foregoing.

Therapeutic compounds/agents or other therapeutic agent(s) or mixtures thereof can be formulated in a carrier system. The carrier can be a colloidal system. The carrier or colloidal system can be a liposome, a phospholipid bilayer vehicle. In one embodiment, therapeutic compound(s)/agent(s) disclosed herein or other therapeutic agent(s) or mixtures thereof can be encapsulated in a liposome while maintaining integrity of the therapeutic compound(s)/agent(s) or other therapeutic agent(s) or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound or other therapeutic agent or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA), poly lactic/glycolic acid (PLGA) or mixtures thereof. The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt. In some embodiments, the nanoparticles or microparticles can be silica-based or silane-based (See for example: WO2002/080977 entitled: “Biodegradable carrier and method for preparation thereof”).

In some embodiments, the therapeutic compound(s)/agent(s) or other therapeutic agent(s) or mixtures thereof are prepared with carriers that will protect the therapeutic compound(s)/agent(s), other therapeutic agent(s) or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compound(s)/agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom to thereby make it available to the subject. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g. a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient(s) for at least 2 days. In some embodiments, the implant or depot formulation is constructed and arranged to deliver therapeutic or prophylactic levels of the active ingredient(s) for at least 7 days. In some embodiments, the implant or depot formulation is constructed and arranged to deliver therapeutic or prophylactic levels of the active ingredient(s) for at least 14 days. In some embodiments, the implant or depot formulation is constructed and arranged to deliver therapeutic or prophylactic levels of the active ingredient(s) for at least 30 days. In some embodiments, the implant or depot formulation is constructed and arranged to deliver therapeutic or prophylactic levels of the active ingredient(s) for at least 60 days. In some embodiments, the implant or depot formulation is constructed and arranged to deliver therapeutic or prophylactic levels of the active ingredient(s) for at least 90 days. In some embodiments, the implant or depot formulation is constructed and arranged to deliver therapeutic or prophylactic levels of the active ingredient(s) for at least 180 days. In some embodiments, the implant or depot formulation is constructed and arranged to deliver therapeutic or prophylactic levels of the active ingredient(s) for at least one year. In some embodiments, the implant or depot formulation is constructed and arranged to deliver therapeutic or prophylactic levels of the active ingredient(s) for 15-30 days. In some embodiments, the implant or depot formulation is constructed and arranged to deliver therapeutic or prophylactic levels of the active ingredient(s) for 30-60 days. In some embodiments, the implant or depot formulation is constructed and arranged to deliver therapeutic or prophylactic levels of the active ingredient(s) for 60-90 days. In some embodiments, the implant or depot formulation is constructed and arranged to deliver therapeutic or prophylactic levels of the active ingredient(s) for 90-120 days. In some embodiments, the implant or depot formulation is constructed and arranged to deliver therapeutic or prophylactic levels of the active ingredient(s) for 120-180 days. In some embodiments, the implant or depot formulation is constructed and arranged to deliver therapeutic or prophylactic levels of the active ingredient(s) for up to one year. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

### V. Compounds & Compositions Useful for Disease, Disorders and/or Conditions Associated with Ferroptosis (e.g., Friedreich's Ataxia)

(a) Therapeutic Compounds (i.e. Therapeutic Agents)

In some embodiments, the present application pertains to compounds and compositions useful for treating disease, disorders and/or conditions associated with ferroptosis (such as Friedreich's ataxia) in a mammalian subject. Said compositions can be medicaments formulated using said compounds and/or other therapeutic agents. Said compositions (comprising said compounds) can be prepared by dissolving, suspending or mixing one or more of the compounds in/with water, solvent, diluent, buffer, adjuvant, vehicle, excipient and/or pharmaceutically acceptable carrier.

In some embodiments, said compound is 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8) having Formula (X);

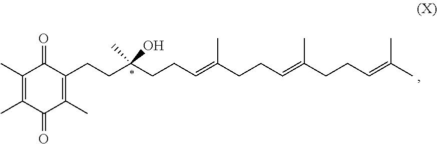 [SMILES: CC(C)=CCC/C(C)=C/CC/C(C)=C/CC[C@](C)(O)CCC1=C(C)C(=O)C(C)=C(C)C1=O]

or a pharmaceutically acceptable salt, hydrate, solvate, and/or tautomer thereof and said compositions comprise said compound, 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8)) or a pharmaceutically acceptable salt, hydrate, solvate, and/or tautomer thereof. Whether alone or used in a composition, Compound 1-1 is generally enantiomerically pure.

In some embodiments, said compound is 2-((S,6E,10E)-3-hydroxy-3,7,11,15-tetramethylhexadeca-6,10,14-trien-1-yl)-3,5,6-trimethylbenzene-1,4-diol (Compound 1-2; No Known CAS#) having Formula (I);

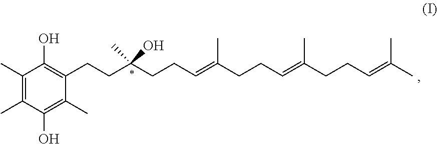 [SMILES: CC(C)=CCC/C(C)=C/CC/C(C)=C/CC[C@](C)(O)CCc1c(C)c(O)c(C)c(C)c1O]

or a pharmaceutically acceptable salt, hydrate, solvate, and/or tautomer thereof and said compositions comprise said compound, 2-((S,6E,10E)-3-hydroxy-3,7,11,15-tetramethylhexadeca-6,10,14-trien-1-yl)-3,5,6-trimethylbenzene-1,4-diol (Compound 1-2; No Known CAS#) or a pharmaceutically acceptable salt, hydrate, solvate, and/or tautomer thereof. Whether alone or used in a composition, Compound 1-2 is generally enantiomerically pure.

In some embodiments, said compound is (R,E)-2-(3-hydroxy-3,7,11-trimethyldodeca-6,10-dien-1-yl)-3,5,6-trimethylcyclohexa-2,5-diene-1,4-dione (Compound 2-1; No Known CAS#) having Formula (II);

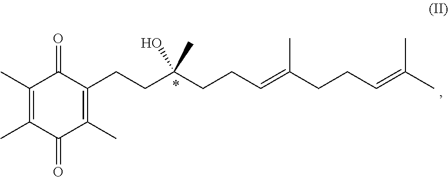 [SMILES: CC(C)=CCC/C(C)=C/CC[C@@](C)(O)CCC1=C(C)C(=O)C(C)=C(C)C1=O]

or a pharmaceutically acceptable salt, hydrate, solvate, and/or tautomer thereof and said compositions comprise said compound, (R,E)-2-(3-hydroxy-3,7,11-trimethyldodeca-6,10-dien-1-yl)-3,5,6-trimethylcyclohexa-2,5-diene-1,4-dione (Compound 2-1; No Known CAS#) or a pharmaceutically acceptable salt, hydrate, solvate, and/or tautomer thereof. Whether alone or used in a composition, Compound 2-1 is generally enantiomerically pure.

In some embodiments, said compound is (R,E)-2-(3-hydroxy-3,7,11-trimethyldodeca-6,10-dien-1-yl)-3,5,6-trimethylbenzene-1,4-diol (Compound 2-2; No Known CAS#) having Formula (III);

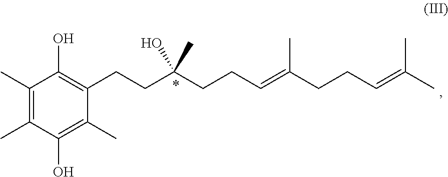 [SMILES: CC(C)=CCC/C(C)=C/CC[C@@](C)(O)CCc1c(C)c(O)c(C)c(C)c1O]

or a pharmaceutically acceptable salt, hydrate, solvate, and/or tautomer thereof and said compositions comprise said compound, (R,E)-2-(3-hydroxy-3,7,11-trimethyldodeca-6,10-dien-1-yl)-3,5,6-trimethylbenzene-1,4-diol (Compound 2-2; No Known CAS#) or a pharmaceutically acceptable salt, hydrate, solvate, and/or tautomer thereof. Whether alone or used in a composition, Compound 2-2 is generally enantiomerically pure.

In some embodiments, said compound is (S,E)-2-(3-hydroxy-3,7,11-trimethyldodeca-6,10-dien-1-yl)-3,5,6-trimethylcyclohexa-2,5-diene-1,4-dione (Compound 3-1; No Known CAS#) having Formula (IV);

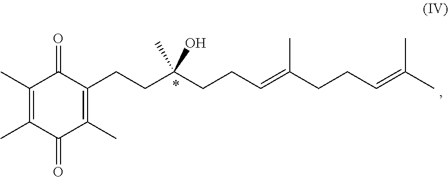 [SMILES: CC(C)=CCC/C(C)=C/CC[C@](C)(O)CCC1=C(C)C(=O)C(C)=C(C)C1=O]

or a pharmaceutically acceptable salt, hydrate, solvate, and/or tautomer thereof and said compositions comprise said compound, (S,E)-2-(3-hydroxy-3,7,11-trimethyldodeca-6,10-dien-1-yl)-3,5,6-trimethylcyclohexa-2,5-diene-1,4-dione (Compound 3-1; No Known CAS#) or a pharmaceutically acceptable salt, hydrate, solvate, and/or tautomer thereof. Whether alone or used in a composition, Compound 3-1 is generally enantiomerically pure.

In some embodiments, said compound is (S,E)-2-(3-hydroxy-3,7,11-trimethyldodeca-6,10-dien-1-yl)-3,5,6-trimethylbenzene-1,4-diol (Compound 3-2; No Known CAS#) having Formula (V);

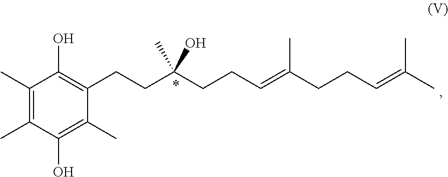 [SMILES: CC(C)=CCC/C(C)=C/CC[C@](C)(O)CCc1c(C)c(O)c(C)c(C)c1O]

or a pharmaceutically acceptable salt, hydrate, solvate, and/or tautomer thereof and said compositions comprise said compound, (S,E)-2-(3-hydroxy-3,7,11-trimethyldodeca-6,10-dien-1-yl)-3,5,6-trimethylbenzene-1,4-diol (Compound 3-2; No Known CAS#) or a pharmaceutically acceptable salt, hydrate, solvate, and/or tautomer thereof. Whether alone or used in a composition, Compound 3-2 is generally enantiomerically pure.

In some embodiments, said compound is (R)-2-(3-hydroxy-3,7-dimethyloct-6-en-1-yl)-3,5,6-trimethylcyclohexa-2,5-diene-1,4-dione (Compound 4-1; No Known CAS#) having Formula (VI);

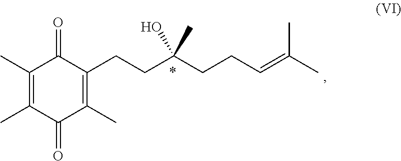 [SMILES: CC(C)=CCC[C@@](C)(O)CCC1=C(C)C(=O)C(C)=C(C)C1=O]

or a pharmaceutically acceptable salt, hydrate, solvate, and/or tautomer thereof and said compositions comprise said compound, (R)-2-(3-hydroxy-3,7-dimethyloct-6-en-1-y1)-3,5,6-trimethylcyclohexa-2,5-diene-1,4-dione (Compound 4-1; No Known CAS#) or a pharmaceutically acceptable salt, hydrate, solvate, and/or tautomer thereof. Whether alone or used in a composition, Compound 4-1 is generally enantiomerically pure.

In some embodiments, said compound is (R)-2-(3-hydroxy-3,7-dimethyloct-6-en-1-yl)-3,5,6-trimethylbenzene-1,4-diol (Compound 4-2; CAS# 116119-16-5) having Formula (Y);

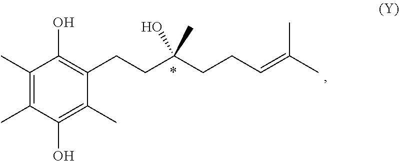 [SMILES: CC(C)=CCC[C@@](C)(O)CCc1c(C)c(O)c(C)c(C)c1O]

or a pharmaceutically acceptable salt, hydrate, solvate, and/or tautomer thereof and said compositions comprise said compound, (R)-2-(3-hydroxy-3,7-dimethyloct-6-en-1-yl)-3,5,6-trimethylbenzene-1,4-diol (Compound 4-2; CAS# 116119-16-5) or a pharmaceutically acceptable salt, hydrate, solvate, and/or tautomer thereof. Whether alone or used in a composition, Compound 4-2 is generally enantiomerically pure.

In some embodiments, said compound is (S)-2-(3-hydroxy-3,7-dimethyloct-6-en-1-yl)-3,5,6-trimethylcyclohexa-2,5-diene-1,4-dione (Compound 5-1; No Known CAS#) having Formula (VII);

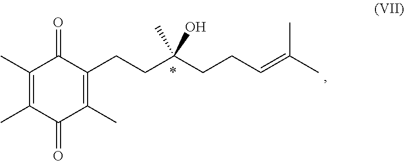 [SMILES: CC(C)=CCC[C@](C)(O)CCC1=C(C)C(=O)C(C)=C(C)C1=O]

or a pharmaceutically acceptable salt, hydrate, solvate, and/or tautomer thereof and said compositions comprise said compound, (S)-2-(3-hydroxy-3,7-dimethyloct-6-en-1-y1)-3,5,6-trimethylcyclohexa-2,5-diene-1,4-dione (Compound 5-1; No Known CAS#) or a pharmaceutically acceptable salt, hydrate, solvate, and/or tautomer thereof. Whether alone or used in a composition, Compound 5-1 is generally enantiomerically pure.

In some embodiments, said compound is (S)-2-(3-hydroxy-3,7-dimethyloct-6-en-1-yl)-3,5,6-trimethylbenzene-1,4-diol (Compound 5-2; No Known CAS#) having Formula (VIII);

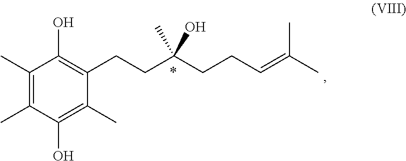 [SMILES: CC(C)=CCC[C@](C)(O)CCc1c(C)c(O)c(C)c(C)c1O]

or a pharmaceutically acceptable salt, hydrate, solvate, and/or tautomer thereof and said compositions comprise said compound, (S)-2-(3-hydroxy-3,7-dimethyloct-6-en-1-yl)-3,5,6-trimethylbenzene-1,4-diol (Compound 5-2; No Known CAS#) or a pharmaceutically acceptable salt, hydrate, solvate, and/or tautomer thereof. Whether alone or used in a composition, Compound 5-2 is generally enantiomerically pure.

Thus, in some embodiments, this application pertains to novel compounds: Compound 1-2 (of Formula I), Compound 2-1 (of Formula II), Compound 2-2 (of Formula III), Compound 3-1 (of Formula IV), Compound 3-2 (of Formula V), Compound 4-1 (of Formula VI), Compound 5-1 (of Formula VII) and Compound 5-2 (of Formula VIII), all of which may be useful in the treatment of disease, disorders and/or conditions associated with ferroptosis.

Further, in some embodiments, this application pertains to compositions (e.g. formulations or medicaments) comprising one or more of Compounds 1-1, 1-2, 2-1, 2-2, 3-1, 3-2, 4-1, 4-2, 5-1 and 5-2, all of which can be formulated in any way suitable for administration to the subject (said formulation being a composition or medicament) for use in the treatment, prevention, inhibition, amelioration or delay in the onset of disease, disorders and/or conditions associated with ferroptosis. All such compositions (e.g. formulations or medicaments) are believed to be novel and inventive because they can be administered for use in the treatment, prevention, inhibition, amelioration or delay in the onset of disease, disorders and/or conditions associated with ferroptosis.

Various possible modes of administration have been previously discussed and all possible modes of administration are contemplated by the present disclosure. Said compounds and compositions can, for example, be formulated as a tablet (for oral administration) or in solution for subcutaneous injection or intravenous injection. In some embodiments, said compounds or compositions can, for example, be prepared as a depot formulation. In some embodiments, said compounds or compositions can, for example, be prepared for ocular treatment, such as formulated into an ointment, eye drops or as a depot formulation for injection into the vitreous of the eye. In some embodiments, said compounds and compositions can, for example, be used to prepare medicaments.

World Intellectual Property Organization (WIPO) published patent application WO 2017/087795 entitled: “Deuterated EPI-743” discloses and claims deuterated versions of vatiquinone and their use as therapeutic agents. More specifically, the published patent application discloses versions of vatiquinone, wherein at least one hydrogen atom (and often substantially more) is substituted with a deuterium atom. Substitution of hydrogen atoms with deuterium atoms does not appear to have any negative effects on the efficacy of the molecule as a therapeutic agent. Accordingly, in some embodiments, this application pertains to deuterated versions of novel compounds: Compound 1-2 (of Formula I), Compound 2-1 (of Formula II), Compound 2-2 (of Formula III), Compound 3-1 (of Formula IV), Compound 3-2 (of Formula V), Compound 4-1 (of Formula VI), Compound 5-1 (of Formula VII) and Compound 5-2 (of Formula VIII), or a pharmaceutically acceptable salt, hydrate, solvate, and/or tautomer thereof, wherein the compound comprises at least one deuterium atom substituted for a hydrogen atom. When a compound disclosed herein is referred to as “substituted”, one or more of the hydrogen atoms of the group has been replaced with a substituent. All such deuterated compounds are believed to be novel compositions of matter and each would be expected to have the same therapeutic efficacy as do unsubstituted compounds (i.e. Compound 1-2 (of Formula I), Compound 2-1 (of Formula II), Compound 2-2 (of Formula III), Compound 3-1 (of Formula IV), Compound 3-2 (of Formula V), Compound 4-1 (of Formula VI), Compound 5-1 (of Formula VII) and Compound 5-2 (of Formula VIII), or a pharmaceutically acceptable salt, hydrate, solvate, and/or tautomer thereof).

### (b) Other Derivatives/Therapeutic Agents

In some embodiments, this application provides for therapeutic compounds that are the reduced forms the hydroquinone versions of Compounds: 1-1, 2-1, 3-1, 4-1 and 5-1 (i.e. Compounds 1-2, 2-2, 3-2, 4-2 and 5-2, respectively). Hydroquinone analogs of known quinone therapeutics used in treating mitochondrial disorders are believed to be suitable for use in treatment, prevention, inhibition, amelioration and delaying onset of a disease, disorder of condition because compounds having a hydroquinone structure, such are vitamin E, have also been shown to be clinically linked with ataxia (See: Imounan et al., Clinical and Genetic Study of Friedreich's ataxia and Ataxia with Vitamin E Deficiency in 44 Moroccan Families, World Journal of Neuroscience, 2014, 4, 299-305; and Abeti et al., Calcium Deregulation: Novel Insights to Understand Friedreich's ataxia Pathophysiology, Frontiers in Cellular Neuroscience: doi: 10.3398/fncel.2018.00264). Furthermore, it is believed that such hydroquinone compounds can themselves be considered therapeutic agents or alternatively as prodrug forms of the quinone therapeutic agents. Specifically, therapeutic quinones are able to enter cells and are believed to be active in the processes affecting the in vivo concentration (e.g., the internal and external mitochondrial concentration) of reactive oxygen species (ROS) and indeed may actively cycle, in vivo, between the reduced form and the oxidized form. Such in vivo cycling between quinone and hydroquinone form is discussed in the literature, for example in Erb et al., PLoSone (April, 2012) 7(4): e36153. In this publication, specific reference is made to compounds having a LogD in the range of 2-7 as being particularly well suited for such in-vivo conversion. For that reason, it is believed that the respective hydroquinones (i.e. Compounds 1-2, 2-2, 3-2, 4-2 and 5-2) should also have therapeutic effect, likely similar effect to what is observed for the respective quinone. Thus, in some embodiments, this application is further directed to novel Compounds 1-2, 2-2, 3-2, and 5-2, as well as any compositions (e.g. formulations or medicaments) comprising any one or more of Compounds 1-2, 2-2, 3-2, 4-2 and 5-2.

Any of the formulations or medicaments disclosed herein could comprise additional therapeutic compounds/agents. The one or more additional therapeutic compounds/agents could be, for example, a beta blocker, ACE inhibitor and/or diuretic used to treat a patient experiencing at risk of heart disease or heart failure. The one or more additional therapeutic compounds could be, for example, a Szeto-Schiller peptide such as SS-20 or SS-31 (a.k.a. SS-31, elamipretide or bendavia). The one or more additional therapeutic compounds could be, for example, a peptidomimetic such as those compounds described in WIPO published patent application WO2019/118878.

### VI. Methods and Uses Related to Compounds 1-1, 1-2, 2-1, 2-2, 3-1, 3-2, 4-1, 4-2, 5-1 and 5-2

### Methods

It has been surprisingly determined that the S-enantiomer (i.e. the opposite enantiomer) of vatiquinone (i.e. 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8)) is active as an anti-ferroptotic agent—which activity appears to be at least equivalent to that of vatiquinone in various test performed and summarized in the Examples section and in Table 1, below. Similarly, novel Compounds 2-1, 3-1, 4-1 and 5-1 exhibit activity as anti-ferroptosis agents. For reasons discussed above, their respective hydroquinones (i.e. Compounds 1-2, 2-2, 3-2, 4-2 and 5-2) are expected to exhibit anti-ferroptosis activity if not in the hydroquinone form then in the quinone form when converted thereto in vivo. Consequently, this application contemplates, based on the data presented, that each of Compounds 1-1, 1-2, 2-1, 2-2, 3-1, 3-2, 4-1, 4-2, 5-1 and 5-2 can be useful in the treatment, prevention, inhibition, amelioration and or delay in the onset of disease, disorders and/or conditions associated with ferroptosis. Similarly, compositions (e.g. formulations or medicaments) comprising one or more of Compounds 1-1, 1-2, 2-1, 2-2, 3-1, 3-2, 4-1, 4-2, 5-1 and 5-2 can be useful in the treatment, prevention, inhibition, amelioration and or delay in the onset of disease, disorders and/or conditions associated with ferroptosis (e.g. Friedreich's ataxia). Hence, said compounds and compositions can be used in methods for treating, preventing, inhibiting, ameliorating and or delay in the onset of disease, disorders and/or conditions associated with ferroptosis (e.g. Friedreich's ataxia).

Compounds with anti-ferroptosis activity are believed to be useful in addressing various diseases, disorders and/or conditions associated an inability to properly regulate cellular iron concentration and/or metabolism as well as forming and/or maintaining iron containing structures/proteins. That Compound 1-1 appears to be at least equivalent to vatiquinone is surprising given that vatiquinone has been known for almost 20 years and been investigated in the clinic for almost 9 years but apparently its stereoisomer has not been given similar consideration as a possible therapeutic agent. Further, given the propensity for in vivo transition between quinone and hydroquinone forms (See: Erb et al., infra), it is expected that administration of 2-((S,6E,10E)-3-hydroxy-3,7,11,15-tetramethylhexadeca-6,10,14-trien-1-yl)-3,5,6-trimethylbenzene-1,4-diol (Compound 1-2; No known CAS#) may be equally effective as is the administration of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8) or vatiquinone. Based on other data presented herein, the same is expected of Compounds 2-1, 2-2, 3-1, 3-2, 4-1, 4-2, 5-1 and 5-2.

Thus, in some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of 2-((S,6E,10E)-3-hydroxy-3,7,11,15-tetramethylhexadeca-6,10,14-trien-1-yl)-3,5,6-rimethylbenzene-1,4-diol (Compound 1-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (R,E)-2-(3-hydroxy-3,7,11-trimethyldodeca-6,10-dien-1-yl)-3,5,6-trimethylcyclohexa-2,5-diene-1,4-dione (Compound 2-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (R,E)-2-(3-hydroxy-3,7,11-trimethyldodeca-6,10-dien-1-yl)-3,5,6-trimethylbenzene-1,4-diol (Compound 2-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (S,E)-2-(3-hydroxy-3,7,11-trimethyldodeca-6,10-dien-1-yl)-3,5,6-trimethylcyclohexa-2,5-diene-1,4-dione (Compound 3-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (S,E)-2-(3-hydroxy-3,7,11-trimethyldodeca-6,10-dien-1-yl)-3,5,6-trimethylbenzene-1,4-diol (Compound 3-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (R)-2-(3-hydroxy-3,7-dimethyloct-6-en-1-yl)-3,5,6-trimethylcyclohexa-2,5-diene-1,4-dione (Compound 4-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (R)-2-(3-hydroxy-3,7-dimethyloct-6-en-1-yl)-3,5,6-trimethylbenzene-1,4-diol (Compound 4-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (S)-2-(3-hydroxy-3,7-dimethyloct-6-en-1-yl)-3,5,6-trimethylcyclohexa-2,5-diene-1,4-dione (Compound 5-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of (S)-2-(3-hydroxy-3,7-dimethyloct-6-en-1-yl)-3,5,6-trimethylbenzene-1,4-diol (Compound 5-2), or a pharmaceutically acceptable salt, hydrate, solvate, and/or tautomer thereof. In some embodiments, the mammalian subject is a human.

Regardless of the indication, any one or more of Compounds 1-1, 1-1, 1-2, 2-1, 2-2, 3-1, 3-2, 4-1, 4-2, 5-1 and 5-2 can be prepared in a composition such as a formulation or medicament prior to administration. The composition can, for example, be prepared by mixing (e.g. to prepare a solution, suspension or dry mixture) any one or more of Compounds 1-1, 1-2, 2-1, 2-2, 3-1, 3-2, 4-1, 4-2, 5-1 and/or 5-2 with water, buffer, diluent, solvent, adjuvant, excipient, vehicle or a pharmaceutically acceptable carrier. Thus, in some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising 2-((S,6E,10E)-3-hydroxy-3,7,11,15-tetramethylhexadeca-6,10,14-trien-1-yl)-3,5,6-trimethylbenzene-1,4-diol (Compound 1-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising (R,E)-2-(3-hydroxy-3,7,11-trimethyldodeca-6,10-dien-1-yl)-3,5,6-trimethylcyclohexa-2,5-diene-1,4-dione (Compound 2-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising (R,E)-2-(3-hydroxy-3,7,11-trimethyldodeca-6,10-dien-1-yl)-3,5,6-trimethylbenzene-1,4-diol (Compound 2-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. medicament) comprising (S,E)-2-(3-hydroxy-3,7,11-trimethyldodeca-6,10-dien-1-yl)-3,5,6-trimethylcyclohexa-2,5-diene-1,4-dione (Compound 3-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising (S,E)-2-(3-hydroxy-3,7,11-trimethyldodeca-6,10-dien-1-yl)-3,5,6-trimethylbenzene-1,4-diol (Compound 3-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising (R)-2-(3-hydroxy-3,7-dimethyloct-6-en-1-yl)-3,5,6-trimethylcyclohexa-2,5-diene-1,4-dione (Compound 4-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising (R)-2-(3-hydroxy-3,7-dimethyloct-6-en-1-yl)-3,5,6-trimethylbenzene-1,4-diol (Compound 4-2), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising (S)-2-(3-hydroxy-3,7-dimethyloct-6-en-1-yl)-3,5,6-trimethylcyclohexa-2,5-diene-1,4-dione (Compound 5-1), or a pharmaceutically acceptable salt, hydrate, solvate, and/or tautomer thereof. In some embodiments, this application pertains to a method for treating, preventing, inhibiting, ameliorating or delaying the onset of a disease, disorder and/or condition associated with ferroptosis in a mammalian subject suffering from a said disease, disorder and/or condition comprising administering to the subject a therapeutically effective amount of a composition (e.g. formulation or medicament) comprising (S)-2-(3-hydroxy-3,7-dimethyloct-6-en-1-yl)-3,5,6-trimethylbenzene-1,4-diol (Compound 5-2), or a pharmaceutically acceptable salt, hydrate, solvate, and/or tautomer thereof. In some embodiments the composition comprises two or more of Compounds 1-1, 1-2, 2-1, 2-2, 3-1, 3-2, 4-1, 4-2, 5-1 and 5-2. In some embodiments, the mammalian subject is a human.

The disease, disorder and/or condition to be addressed by administration of Compounds 1-1, 1-2, 2-1, 2-2, 3-1, 3-2, 4-1, 4-2, 5-1 and/or 5-2 (or a composition comprising one or more of Compounds 1-1, 1-2, 2-1, 2-2, 3-1, 3-2, 4-1, 4-2, 5-1 and 5-2) can be Friedrich's ataxia. The disease, disorder and/or condition to be addressed can be Leigh syndrome. The disease, disorder and/or condition to be addressed can be Leber's Hereditary Optic Neuropathy (LHON). The disease, disorder and/or condition to be addressed can be (proliferative, non-proliferative, diabetic or hypertensive) retinopathy. The disease, disorder and/or condition to be addressed can be refractory epilepsy. The disease, disorder and/or condition to be addressed can be a neurological disorder such as Parkinson's disease, Alzheimer's disease, Huntington's disease or Amyotrophic Lateral Sclerosis (ALS). The disease, disorder and/or condition to be addressed can be a cardiomyopathy (e.g. cardiac ischemia-reperfusion injury, myocardial infarction or heart failure). The disease, disorder and/or condition to be addressed can be a renal disorder such as renal injury, renal ischemia reperfusion injury or acute renal failure.

Compounds 1-1, 1-2, 2-1, 2-2, 3-1, 3-2, 4-1, 4-2, 5-1 and/or 5-2 (or a composition comprising one or more of Compounds 1-1, 1-2, 2-1, 2-2, 3-1, 3-2, 4-1, 4-2, 5-1 and 5-2) can be administered by any suitable route of administration, including: orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly. In some embodiments, administration of the compound or composition is oral. In some embodiments, administration of the compound or composition is intraocular. In some embodiments, administration of the composition is systemic. In some embodiments, administration of the composition is intravenous.

Administration of Compounds 1-1, 1-2, 2-1, 2-2, 3-1, 3-2, 4-1, 4-2, 5-1 and/or 5-2 (or a composition comprising one or more of Compounds 1-1, 1-2, 2-1, 2-2, 3-1, 3-2, 4-1, 4-2, 5-1 and 5-2) can be performed for any suitable duration, such as administration for: 6 weeks or more, 12 weeks or more, 24 weeks or more, 48 weeks or more, 96 weeks or more, 1 year or more, 2 years or more, 5 years or more or 10 years or more. In some embodiments, the compound or composition can be administered from the time of diagnosis or shortly after diagnosis until the subject dies or until its administration is no longer effective. Compounds 1-1, 1-2, 2-1, 2-2, 3-1, 3-2, 4-1, 4-2, 5-1 and/or 5-2 (or a composition comprising one or more of Compounds 1-1, 1-2, 2-1, 2-2, 3-1, 3-2, 4-1, 4-2, 5-1 and 5-2) can be administered by oneself, another or by an instrument/device such as an infusion pump.

In some embodiments, this application pertains to a method for treating, preventing, inhibiting, ameliorating or delaying the onset of Friedreich's ataxia in a mammalian subject suffering from a said disease, comprising administering to the subject a therapeutically effective amount of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of 2-((S,6E,10E)-3-hydroxy-3,7,11,15-tetramethylhexadeca-6,10,14-trien-1-yl)-3,5,6-trimethylbenzene-1,4-diol (Compound 1-2), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of (R,E)-2-(3-hydroxy-3,7,11-trimethyldodeca-6,10-dien-1-yl)-3,5,6-trimethylcyclohexa-2,5-diene-1,4-dione (Compound 2-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of (R,E)-2-(3-hydroxy-3,7,11-trimethyldodeca-6,10-dien-1-yl)-3,5,6-trimethylbenzene-1,4-diol (Compound 2-2), or a pharmaceutically acceptable salt, hydrate, solvate, and/or tautomer thereof. In some embodiments, this application pertain to a method for treating, preventing, inhibiting, ameliorating or delaying the onset of Friedreich's ataxia in a mammalian subject suffering from a said disease, comprising administering to the subject a therapeutically effective amount of (S,E)-2-(3-hydroxy-3,7,11-trimethyldodeca-6,10-dien-1-yl)-3,5,6-trimethylcyclohexa-2,5-diene-1,4-dione (Compound 3-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of (S,E)-2-(3-hydroxy-3,7,11-trimethyldodeca-6,10-dien-1-yl)-3,5,6-trimethylbenzene-1,4-diol (Compound 3-2), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of (R)-2-(3-hydroxy-3,7-dimethyloct-6-en-1-yl)-3,5,6-trimethylcyclohexa-2,5-diene-1,4-dione (Compound 4-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of (R)-2-(3-hydroxy-3,7-dimethyloct-6-en-1-yl)-3,5,6-trimethylbenzene-1,4-diol (Compound 4-2), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of (S)-2-(3-hydroxy-3,7-dimethyloct-6-en-1-yl)-3,5,6-trimethylcyclohexa-2,5-diene-1,4-dione (Compound 5-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease, comprising administering to the subject a therapeutically effective amount of (S)-2-(3-hydroxy-3,7-dimethyloct-6-en-1-yl)-3,5,6-trimethylbenzene-1,4-diol (Compound 5-2), or a pharmaceutically acceptable salt, hydrate, solvate, and/or tautomer thereof. In some embodiments, the mammalian subject is a human.

Any one or more of Compounds 1-1, 1-1, 1-2, 2-1, 2-2, 3-1, 3-2, 4-1, 4-2, 5-1 and 5-2 can be, prior to administration, prepared in a composition such as a formulation or medicament for treatment of Friedreich's ataxia. The composition can, for example, be prepared by mixing (e.g. to prepare a solution, suspension or dry mixture) any one or more of Compounds 1-1, 1-2, 2-1, 2-2, 3-1, 3-2, 4-1, 4-2, 5-1 and/or 5-2 with water, buffer, diluent, solvent, adjuvant, excipient, vehicle or a pharmaceutically acceptable carrier. Thus, in some embodiments, the compound is formulated into a composition or medicament. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising 2-[(3S,6E, 10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising 2-((S,6E,10E)-3-hydroxy-3,7,11,15-tetramethylhexadeca-6,10,14-trien-1-yl)-3,5,6-trimethylbenzene-1,4-diol (Compound 1-2), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (R,E)-2-(3-hydroxy-3,7,11-trimethyldodeca-6,10-dien-1-yl)-3,5,6-trimethylcyclohexa-2,5-diene-1,4-dione (Compound 2-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (R,E)-2-(3-hydroxy-3,7,11-trimethyldodeca-6,10-dien-1-yl)-3,5,6-trimethylbenzene-1,4-diol (Compound 2-2), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (S,E)-2-(3-hydroxy-3,7,11-trimethyldodeca-6,10-dien-1-yl)-3,5,6-trimethylcyclohexa-2,5-diene-1,4-dione (Compound 3-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (S,E)-2-(3-hydroxy-3,7,11-trimethyldodeca-6,10-dien-1-yl)-3,5,6-trimethylbenzene-1,4-diol (Compound 3-2), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (R)-2-(3-hydroxy-3,7-dimethyloct-6-en-1-yl)-3,5,6-trimethylcyclohexa-2,5-diene-1,4-dione (Compound 4-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (R)-2-(3-hydroxy-3,7-dimethyloct-6-en-1-yl)-3,5,6-trimethylbenzene-1,4-diol (Compound 4-2), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (S)-2-(3-hydroxy-3,7-dimethyloct-6-en-1-yl)-3,5,6-trimethylcyclohexa-2,5-diene-1,4-dione (Compound 5-1), or a pharmaceutically acceptable salt, hydrate, solvate, and/or tautomer thereof. In some embodiments, this application pertains to a method for treating, preventing, inhibiting, ameliorating or delaying the onset of Friedreich's ataxia in a mammalian subject suffering from a said disease comprising administering to the subject a therapeutically effective amount of a composition (e.g. formulation or medicament) comprising (S)-2-(3-hydroxy-3,7-dimethyloct-6-en-1-yl)-3,5,6-trimethylbenzene-1,4-diol (Compound 5-2), or a pharmaceutically acceptable salt, hydrate, solvate, and/or tautomer thereof. In some embodiments the composition comprises two or more of Compounds 1-1, 1-2, 2-1, 2-2, 3-1, 3-2, 4-1, 4-2, 5-1 and 5-2. In some embodiments, the mammalian subject is a human.

The aforementioned compounds or compositions (e.g. formulations or medicaments) comprising one or more of Compounds 1-1, 1-1, 1-2, 2-1, 2-2, 3-1, 3-2, 4-1, 4-2, 5-1 and 5-2) suitable for administration to subjects having, or suspected of having, Friedreich's ataxia can be administered by any suitable route of administration, including: orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly. In some embodiments, administration of the compound or composition is oral. In some embodiments, administration of the compound or composition is intraocular. In some embodiments, administration of the composition is systemic. In some embodiments, administration of the composition is intravenous.

Administration of compositions comprising one or more of Compounds 1-1, 1-1, 1-2, 2-1, 2-2, 3-1, 3-2, 4-1, 4-2, 5-1 and 5-2) can be performed for any suitable duration, such as administration for: 6 weeks or more, 12 weeks or more, 24 weeks or more, 48 weeks or more, 96 weeks or more, 1 year or more, 2 years or more, 5 years or more or 10 years or more. In some embodiments, the composition can be administered from the time of diagnosis or shortly after diagnosis until the subject dies or until its administration is no longer effective. Compounds 1-1, 1-2, 2-1, 2-2, 3-1, 3-2, 4-1, 4-2, 5-1 and/or 5-2 (or a composition comprising one or more of Compounds 1-1, 1-1, 1-2, 2-1, 2-2, 3-1, 3-2, 4-1, 4-2, 5-1 and 5-2) can be administered by oneself, another or by an instrument/device such as an infusion pump.

### Related Uses

This application further pertains to the use of a pharmaceutical composition or medicament for treating, preventing, inhibiting, ameliorating or delaying the onset of a disease, disorder or condition associated with ferroptosis in a mammalian subject. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1). In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises 2-((S,6E,10E)-3-hydroxy-3,7,11,15-tetramethylhexadeca-6,10,14-trien-1-yl)-3,5,6-trimethylbenzene-1,4-diol (Compound 1-2), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (R,E)-2-(3-hydroxy-3,7,11-trimethyldodeca-6,10-dien-1-yl)-3,5,6-trimethylcyclohexa-2,5-diene-1,4-dione (Compound 2-1), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (R,E)-2-(3-hydroxy-3,7,11-trimethyldodeca-6,10-dien-1-yl)-3,5,6-trimethylbenzene-1,4-diol (Compound 2-2), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (S,E)-2-(3-hydroxy-3,7,11-trimethyldodeca-6,10-dien-1-yl)-3,5,6-trimethylcyclohexa-2,5-diene-1,4-dione (Compound 3-1), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (S,E)-2-(3-hydroxy-3,7,11-trimethyldodeca-6,10-dien-1-yl)-3,5,6-trimethylbenzene-1,4-diol (Compound 3-2), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (R)-2-(3-hydroxy-3,7-dimethyloct-6-en-1-yl)-3,5,6-trimethylcyclohexa-2,5-diene-1,4-dione (Compound 4-1), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (R)-2-(3-hydroxy-3,7-dimethyloct-6-en-1-yl)-3,5,6-trimethylbenzene-1,4-diol (Compound 4-2), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (S)-2-(3-hydroxy-3,7-dimethyloct-6-en-1-yl)-3,5,6-trimethylcyclohexa-2,5-diene-1,4-dione (Compound 5-1), or a pharmaceutically acceptable salt, hydrate, solvate, and/or tautomer of the forgoing. In some embodiments, this application relates to the use of a pharmaceutical composition or medicament for treating, preventing, inhibiting, ameliorating or delaying the onset of a disease, disorder or condition associated with ferroptosis in a mammalian subject, wherein the pharmaceutical composition or medicament comprises (S)-2-(3-hydroxy-3,7-dimethyloct-6-en-1-yl)-3,5,6-trimethylbenzene-1,4-diol (Compound 5-2), or a pharmaceutically acceptable salt, hydrate, solvate, and/or tautomer of the forgoing. In some embodiments, the mammalian subject is human.

The disease, disorder and/or condition associated with the aforementioned use can be Friedrich's ataxia. The disease disorder and/or condition associated with the aforementioned use can be Leigh syndrome. The disease, disorder and/or condition associated with the aforementioned use can be Leber's Hereditary Optic Neuropathy (LHON). The disease, disorder and/or condition associated with the aforementioned use can be (proliferative, non-proliferative, diabetic or hypertensive) retinopathy. The disease, disorder and/or condition associated with the aforementioned use can be refractory epilepsy. The disease, disorder and/or condition associated with the aforementioned use can be a neurological disorder such as Parkinson's disease, Alzheimer's disease, Huntington's disease or Amyotrophic Lateral Sclerosis (ALS). The disease, disorder and/or condition associated with the aforementioned use can be a cardiomyopathy (e.g. cardiac ischemia-reperfusion injury, myocardial infarction or heart failure). The disease, disorder and/or condition associated with the aforementioned use can be a renal disorder such as renal injury, renal ischemia reperfusion injury or acute renal failure.

With respect to the aforementioned use(s) of the pharmaceutical formulation or medicament comprising one or more of Compounds 1-1, 1-2, 2-1, 2-2, 3-1, 3-2, 4-1, 4-2, 5-1 and/or 5-2 can comprise administration by any suitable route of administration, including: orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly. In some embodiments, administration of the compound or composition is oral. In some embodiments, administration of the compound or composition is intraocular. In some embodiments, administration of the composition is systemic. In some embodiments, administration of the composition is intravenous.

Administration of the pharmaceutical formulation or medicament comprising one or more of Compounds 1-1, 1-1, 1-2, 2-1, 2-2, 3-1, 3-2, 4-1, 4-2, 5-1 and 5-2) can be performed for any suitable duration, such as administration for: 6 weeks or more, 12 weeks or more, 24 weeks or more, 48 weeks or more, 96 weeks or more, 1 year or more, 2 years or more, 5 years or more or 10 years or more. In some embodiments, the composition can be administered from the time of diagnosis or shortly after diagnosis until the subject dies or until its administration is no longer effective. The pharmaceutical formulations or medicaments comprising one or more of Compounds 1-1, 1-1, 1-2, 2-1, 2-2, 3-1, 3-2, 4-1, 4-2, 5-1 and 5-2) can be administered by oneself, another or by an instrument/device such as an infusion pump.

### VII. Method For Making 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8) as well as Compounds 2-1, 3-1, 4-1 and 5-1

2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1) is a known compound having a Chemical Abstract Services Number of 1213269-99-8. However, this chemical composition of matter appears to only have ever been described in WO 2010/030607 (and related counterpart national phase applications). Its synthesis was not reported in said published WIPO patent application.

2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1; CAS# 1213269-99-8) can be (and was) prepared by adaptation of the processes described in published WIPO application WO2021/067836 as more fully described in Example 1, below. In brief, the identical process was used as was described in WO 2010/030607 to prepare vatiquinone (CAS# 1213269-98-7), except that the other enantiomer (i.e. 11a instead of 11b) isolated at Step. e was used in subsequent steps.

This process was extended to prepare Compounds 2-1, 3-1, 4-1 and 5-1, by use of compounds 13a or 13b and the appropriate tail molecule as illustrated in the Examples, below. The detailed synthesis of all of Compounds 1-1, 2-1, 3-1, 4-1 and 5-1 are described in the Examples, below.

### VIII. Method For Making 2-((S,6E,10E)-3-hydroxy-3,7,11,15-tetramethylhexadeca-6,10,14-trien-1-yl)-3,5,6-trimethylbenzene-1,4-diol (Compound 1-2; CAS# Not Known) as well as Compounds 2-2, 3-2, 4-2 and 5-2

2-((S,6E,10E)-3-hydroxy-3,7,11,15-tetramethylhexadeca-6,10,14-trien-1-yl)-3,5,6-trimethylbenzene-1,4-diol (Compound 1-2; No known CAS#) appears to be a novel compound. Compound 1-2 can however be prepared in one step starting from 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1) by following the process for reducing vatiquinone to its reduced form as described in published WIPO application—WO2021/067836 (Example 8, Scheme 9). This process has been observed by applicants to be robust in the conversion of other quinones to hydroquinone equivalents with the caveat that often the hydroquinone compounds are rapidly oxidized back to the quinone form when exposed to molecular O₂ (i.e. air). Generally, to maintain the hydroquinone compound, care must be taken to exclude oxygen (i.e. air) during workup and from containers containing product else the product will oxidize back to the quinone.

### EXAMPLES

### Example 1: Synthesis of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione (Compound 1-1)

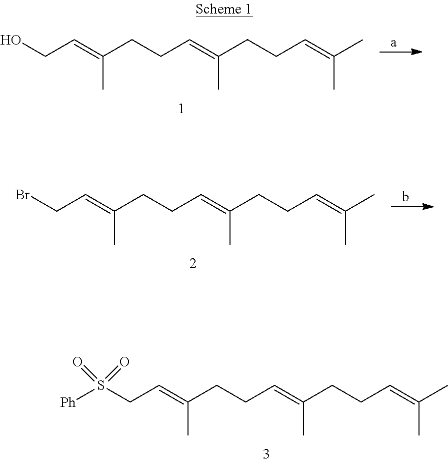 [SMILES: CC(C)=CCC/C(C)=C/CC/C(C)=C/CO ; CC(C)=CCC/C(C)=C/CC/C(C)=C/CBr ; CC(C)=CCC/C(C)=C/CC/C(C)=C/CS(=O)(=O)c1ccccc1]

Step a. Synthesis of (2E,6E)-1-bromo-3,7,11-trimethyldodeca-2,6,10-triene (2)

To a cooled (0° C.) solution of (2E,6E)-3,7,11-trimethyldodeca-2,6,10-trien-1-ol (1, 2 g, 8.99 mmol) in dry tetrahydrofuran (THF, 30 mL) dropwise was added phosphorus tribromide (PBr₃, 1.02 mL, 10.8 mmol) and the reaction mixture was stirred at 0° C. for 1 hour (hr. or h.). The reaction mixture was poured on ice (50 g) and extracted with diethyl ether (Et₂O, 3×80 mL). The combined organic phases were dried over anhydrous (anh.) Na₂SO₄ and concentrated under reduced pressure to give 2 (2.5 g) in 98% yield.

Step b. Synthesis of (((2E,6E)-3,7,11-trimethyldodeca-2,6,10-trien-1-yl)sulfonyl)benzene (3)

To a cooled (0° C.) solution of 2 (2.5 g, 8.81 mmol) in dry N,N-dimethylformamide (DMF, 30 mL) was added phenylsulfinic acid sodium salt (1.89 g, 11.5 mmol) in one portion and the reaction mixture was stirred at room temperature (r.t.) overnight. The reaction mixture was quenched by addition of saturated aqueous (sat. aq.) NH₄Cl (80 mL) and ethyl acetate (EtOAc, 500 mL) and stirred at r.t. for 15 minutes (min.). The aqueous phase was removed and the organic phase was washed with saturated NaCl (brine, 4×80 mL), dried over anh. Na₂SO₄ and concentrated under reduced pressure. The crude product was purified by silica gel flash column chromatography using a mixture of petroleum ether and EtOAc (1:0 →10:1) as an eluent to give 3 (1.36 g) in 45% yield.

¹H-NMR (CDCl₃, 400 MHZ): δ=7.90-7.83 (m, 2H), 7.68-7.48 (m, 3H), 5.24-5.00 (m, 3H), 3.84-3.77 (m, 2H), 2.11-1.93 (m, 8H), 1.62-1.53 (m, 9H), 1.32-1.29 ppm (m, 3H).

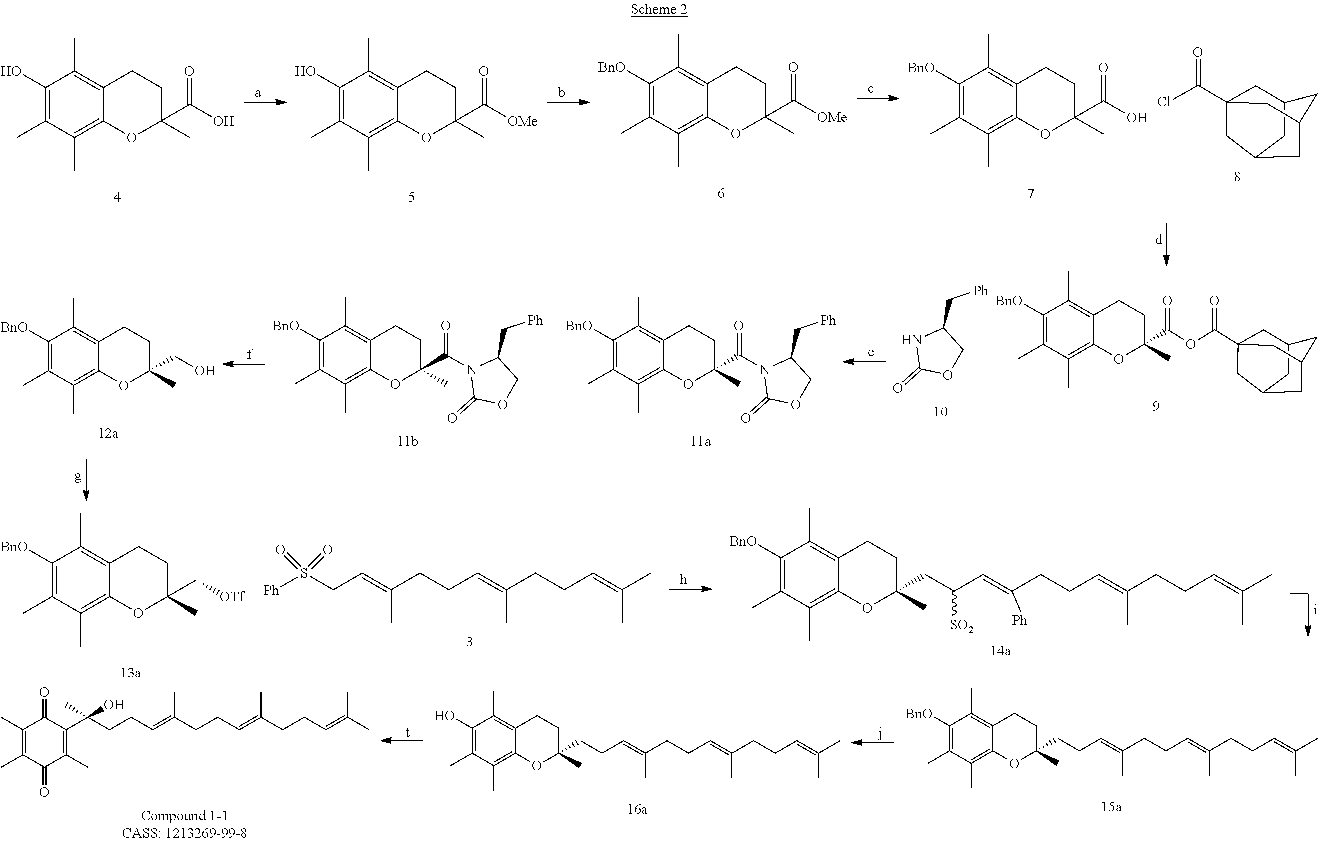

Step a. Synthesis of Methyl 6-hydroxy-2,5,7,8-tetramethylchromane-2-carboxylate (5)

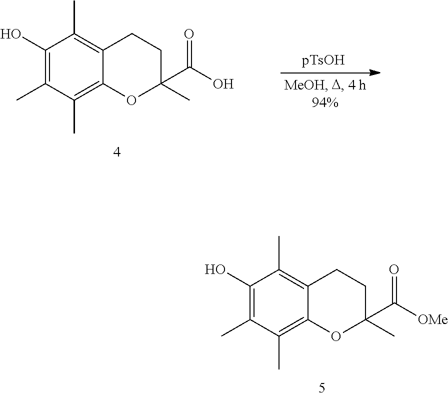 [SMILES: Cc1c(C)c2c(c(C)c1O)CCC(C)(C(=O)O)O2 ; COC(=O)C1(C)CCc2c(C)c(O)c(C)c(C)c2O1]

To a mixture of 4 (10 g, 40 mmol) and p-toluenesulfonic acid (3.8 g, 20 mmol) was added dry methanol (80 mL) and the reaction mixture was stirred under reflux for 4 hr. After cooling to r.t., methanol (MeOH) was removed under reduced pressure. Then, EtOAc (600 mL) and sat. aq. sodium bicarbonate (Na₂CO₃, 200 mL) were added and the resulting mixture was stirred at r.t. for 15 min. The aqueous phase was separated and the organic phase was washed with sat. aq. Na₂CO₃ (3×100 mL) and brine (100 mL) and dried over anh. Na₂SO₄. After evaporation of volatile matters under reduced pressure, 5 (9.9 g) was obtained in 94% yield.

¹H-NMR (DMSO-d₆, 400 MHz): δ=7.45 (br s, 1H), 3.59 (s, 3H), 2.62-2.46 (m, 1H), 2.42-2.23 (m, 2H), 2.06 (s, 3H), 2.04 (s, 3H), 1.97 (s, 3H), 1.86-1.71 (m, 1H), 1.51 ppm (s, 3H).

Step b. Synthesis of Methyl 6-(benzyloxy)-2,5,7,8-tetramethylchromane-2-carboxylate (6)

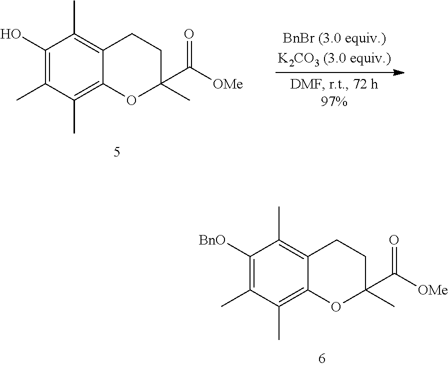 [SMILES: COC(=O)C1(C)CCc2c(C)c(O)c(C)c(C)c2O1 ; COC(=O)C1(C)CCc2c(C)c(OCc3ccccc3)c(C)c(C)c2O1]

To a suspension of 5 (5.0 g, 18.9 mmol) and potassium carbonate (K₂CO₃, 7.8 g, 56.7 mmol) in dry N,N-dimethylformamide (DMF, 30 mL) was added benzyl bromide (6.7 mL, 56.7 mmol) and the reaction mixture was stirred at r.t. for 72 hr. The volatile matters were removed under reduced pressure and the residue was quenched by the addition of EtOAc (600 mL), water (100 mL), and brine (100 mL) and stirred at r.t. for 15 min. After separation of the aqueous phase, the organic phase was washed with brine (3×150 mL), dried over anh. Na₂SO₄, and concentrated under reduced pressure. The crude product was purified by silica gel flash column chromatography using a mixture of petroleum ether and EtOAc (20:1) as an eluent to give 6 (6.5 g) in 97% yield.

¹H-NMR (CDCl₃, 400 MHZ): δ=7.53-7.29 (m, 5H), 4.69 (s, 2H), 3.69 (s, 3H), 2.70-2.38 (m, 3H), 2.23 (s, 3H), 2.18 (s, 3H), 2.13 (s, 3H), 1.94-1.81 (m, 1H), 1.62 ppm (s, 3H).

Step c. Synthesis of 6-(Benzyloxy)-2,5,7,8-tetramethylchromane-2-carboxylic Acid (7)

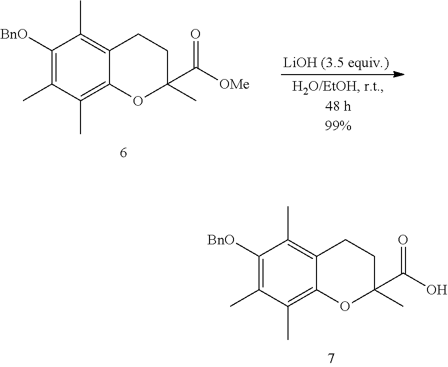 [SMILES: COC(=O)C1(C)CCc2c(C)c(OCc3ccccc3)c(C)c(C)c2O1 ; Cc1c(C)c2c(c(C)c1OCc1ccccc1)CCC(C)(C(=O)O)O2]

To a suspension of 6 (3.6 g, 10.2 mmol) in a mixture of water/ethanol (H₂O/EtOH, 70 mL; 1:2.5) was added lithium hydroxide (LiOH, 1.50 g, 35.7 mmol) and the resulting mixture was stirred at r.t. for 48 hr. After removal of ethanol under reduced pressure, the residual aqueous solution was diluted with water (10 mL) and acidified to pH 2. The resulting white precipitate was filtered, washed with water (3×15 mL), and dried under vacuum to give 7 (3.44 g) in 99% yield.

¹H-NMR (DMSO-d₆, 400 MHZ): δ=7.52-7.29 (m, 5H), 4.62 (s, 2H), 2.66-2.24 (m, 3H), 2.14 (s, 3H), 2.09-2.03 (m, 6H), 1.81-1.68 (m, 1H), 1.51 ppm (s, 3H).

Steps d, e. Synthesis of (S)-4-benzyl-3-((S)-6-(benzyloxy)-2,5,7,8-tetramethylchromane-2-carbonyl)oxazolidin-2-one (Mixture of 11a and 11b)

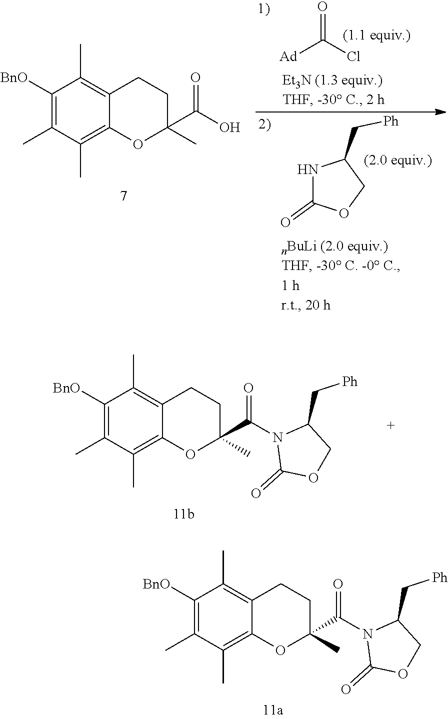

To a cooled (−30° C.) solution of 7 (6.62 g, 19.5 mmol) in dry tetrahydrofuran (THF, 120 mL) under argon atmosphere was added solution of 1-adamantanecarbonyl chloride (4.25 g, 21.4 mmol) in dry THF (20 mL) and triethylamine (Et₃N, 3.5 mL, 25.4 mmol) and the resulting mixture was stirred at the same temperature for 2 hr. Then, the reaction mixture was cooled to −78° C. To a cooled (−78° C.) solution of (S)-4-benzyl-2-oxazolidinone (6.91 g, 39 mmol) in dry THF (100 mL), dropwise was added n-butyl lithium (ⁿBuLi, 2.3 M in hexanes, 17 mL, 39 mmol) and the resulting mixture was transferred by cannula to the activated acid solution over 10 min. Then, the reaction mixture was placed in ice bath and stirred for 1 h. After removal of cooling bath, the reaction mixture was stirred at r.t. for 20 hr. The reaction mixture was quenched by the addition of EtOAc (500 mL) and water (100 mL) and the resulting mixture was stirred at r.t. for 15 min. After separation of the aqueous phase, the organic phase was washed with sat. aq. Na₂CO₃ (3×100 mL) and brine (100 mL), dried over anh. Na₂SO₄, and concentrated under reduced pressure. The obtained crude mixture of diastereomers was separated by silica gel flash column chromatography using a mixture of petroleum ether and EtOAc as an eluent. The required less polar diastereomer 11a came out of the column by PE/EtOAc (40:3), but the more polar 11b by PE/EtOAc (10:1). Thus, 11b (2.9 g) was obtained in 60% yield and the less polar 11a (3.4 g) in 70% yield.

¹H-NMR of 11a: (CDCl₃, 400 MHZ): 7.50-7.15 (m, 10H), 4.68 (s, 2H), 4.34-4.24 (m, 1H), 4.06-3.99 (m, 1H), 3.86-3.78 (m, 1H), 3.35-3.25 (m, 1H), 2.88-2.70 (m, 2H), 2.66-2.56 (m, 2H), 2.21 (s, 3H), 2.17 (s, 3H), 2.13 (s, 3H), 2.02-1.85 (m, 1H), 1.90 ppm (s, 3H).

¹H-NMR of 11b: (CDCl₃, 400 MHZ): δ=7.50-7.17 (m, 8H), 7.09-7.04 (m, 2H), 4.72-4.60 (m, 3H), 4.16-4.08 (m, 1H), 3.95-3.89 (m, 1H), 3.12-3.04 (m, 1H), 2.91-2.82 (m, 1H), 2.69-2.49 (m, 2H), 2.26 (s, 3H), 2.20 (s, 3H), 2.19-2.07 (m, 4H), 2.01-1.91 (m, 1H), 1.87 ppm (s, 3H).

Step f. Synthesis of (R)-(6-(benzyloxy)-2,5,7,8-tetramethylchroman-2-yl)methanol (12a)

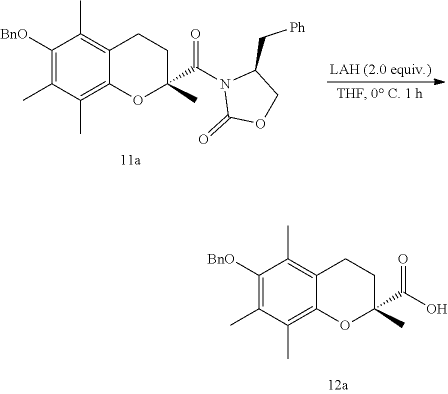 [SMILES: Cc1c(C)c2c(c(C)c1OCc1ccccc1)CC[C@](C)(C(=O)N1C(=O)OC[C@@H]1Cc1ccccc1)O2 ; Cc1c(C)c2c(c(C)c1OCc1ccccc1)CC[C@](C)(C(=O)O)O2]

To a cooled (0° C.) solution of 11a (14.4 g, 28.8 mmol) in dry THF (150 mL) dropwise was added lithium aluminum hydride solution in THF (1 M, 58 mL, 57.6 mmol) and the reaction mixture was stirred at 0° C. for 1 hr. Under continuous cooling, ethanol was added dropwise until excess of lithium aluminum hydride was quenched. Then, 1 M aqueous (aq.) NaOH (230 mL) was added dropwise and stirred at r.t. for 15 min. After re-cooling to 0° C., 1M aq. Hydrochloric acid (HCl) was added until the resulting mixture reached about pH 4. Then, dichloromethane (DCM, 500 mL) was added and the resulting mixture was stirred at r.t. for 15 min. After separation of the organic phase, the aqueous phase was extracted with DCM (2×150 mL). The combined organic phases were washed with brine (150 mL), dried over anh. Na₂SO₄ and concentrated under reduced pressure. The crude product was purified by silica gel flash column chromatography using a mixture of petroleum ether and EtOAc (1:0→40:7) as an eluent to give 12a (7.5 g) in 80% yield.

¹H-NMR (CDCl₃, 400 MHZ): δ=7.53-7.47 (m, 2H), 7.45-7.30 (m, 3H), 4.70 (s, 2H), 3.70-3.57 (m, 2H), 2.70-2.61 (m, 2H), 2.23 (s, 3H), 2.18 (s, 3H), 2.11 (s, 3H), 2.08-1.95 (m, 1H), 1.80-1.69 (m, 1H), 1.25-1.23 ppm (m, 3H).

Step g. Synthesis of (R)-(6-(benzyloxy)-2,5,7,8-tetramethylchroman-2-yl)methyl Trifluoromethanesulfonate (13a)

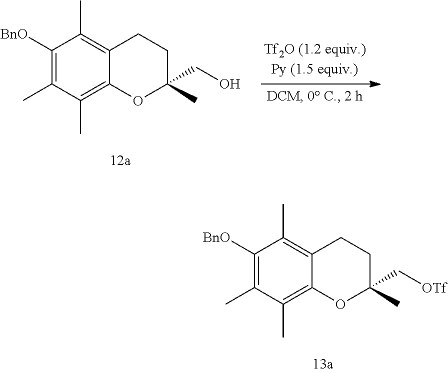 [SMILES: Cc1c(C)c2c(c(C)c1OCc1ccccc1)CC[C@](C)(CO)O2 ; Cc1c(C)c2c(c(C)c1OCc1ccccc1)CC[C@](C)(COS(=O)(=O)C(F)(F)F)O2]

To a solution of 12a (9.1 g, 27.9 mmol) in dry dichloromethane (DCM, 100 mL) was added pyridine (3.4 ml, 41.9 mmol). After cooling to 0° C., trifluoromethanesulfonic anhydride (5.6 mL, 33.5 mmol) was added dropwise and the reaction mixture was stirred at 0° C. for 2 hr. The reaction mixture was quenched by the addition of ice (30 g) and allowed to warm to r.t. Then, EtOAc (600 mL) and water (150 mL) were added and the resulting mixture was stirred at r.t. for 15 min. The aqueous phase was separated and the organic phase was washed with brine (2×150 mL) and dried over anh. Na₂SO₄. After removal of volatile matters, 13a (11.8 g) was obtained in 92% yield.

¹H-NMR (CDCl₃, 400 MHZ): δ=7.55-7.30 (m, 5H), 4.70 (s, 2H), 4.53-4.4 (m, 2H), 2.72-2.61 (m, 2H), 2.23 (s, 3H), 2.18 (s, 3H), 2.10 (s, 3H), 2.04-1.93 (m, 1H), 1.92-1.80 (m, 1H), 1.38 ppm (s, 3H).

Step h. Synthesis of (2R)-6-(benzyloxy)-2,5,7,8-tetramethyl-2-((3E,7E)-4,8,12-trimethyl-2-(phenylsulfonyl)trideca-3,7,11-trien-1-yl)chromane (14a)

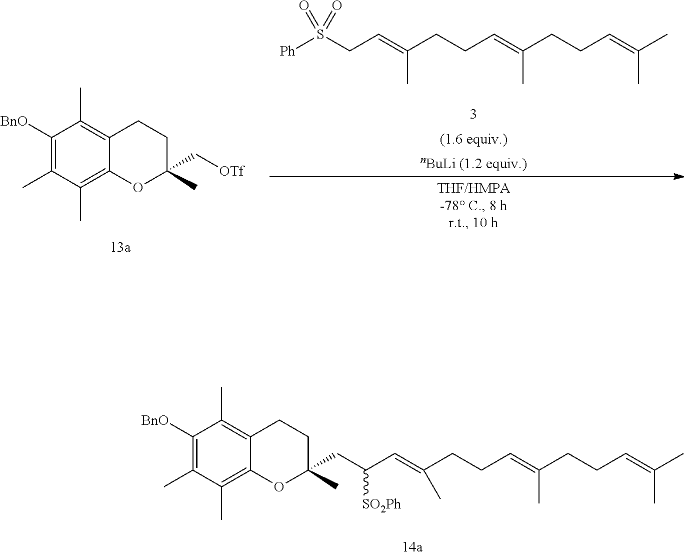

To a solution of 3 (3.64 g, 10.5 mmol, prepared as described in Scheme 1 and the accompanying description) in dry THF (35 mL) under argon was added hexamethylphosphoramide (8.6 mL) and the resulting mixture was cooled to −78° C. Then, ⁿBuLi (2.3 M in hexanes, 3.4 mL, 7.85 mmol) was added dropwise and the reaction mixture was stirred at the same temperature for 30 min. After dropwise addition of 13a (3.00 g, 6.54 mmol) solution in dry THF (35 mL), the reaction mixture was stirred at −78° C. for 8 hr. and stirring was continued at r.t. for 20 hr. The reaction mixture was diluted with Et₂O (500 mL), washed with brine (2×100 mL), dried over anh. Na₂SO₄ and concentrated under reduced pressure. The crude product was purified by silica gel flash column chromatography using a mixture of petroleum ether and EtOAc (1:0→20:3) as an eluent and re-purified by reversed phase flash chromatography (Acetonitrile (MeCN)/H₂O; 80-95%) to give 14a (3.46 g) as a mixture of diastereomers in 81% yield.

¹H-NMR (CDCl₃, 400 MHZ): δ=7.89-7.71 (m, 4H), 7.67-7.28 (m, 16H), 5.23-4.96 (m, 6H), 4.68 (s, 2H), 4.20-3.99 (m, 2H), 2.64-2.45 (m, 4H), 2.23-1.45 (m, 56H), 1.34-1.09 ppm (m, 16H).

Step i. Synthesis of (S)-6-(benzyloxy)-2,5,7,8-tetramethyl-2-((3E,7E)-4,8,12-trimethyltrideca-3,7,11-trien-1-yl)chromane (15a)

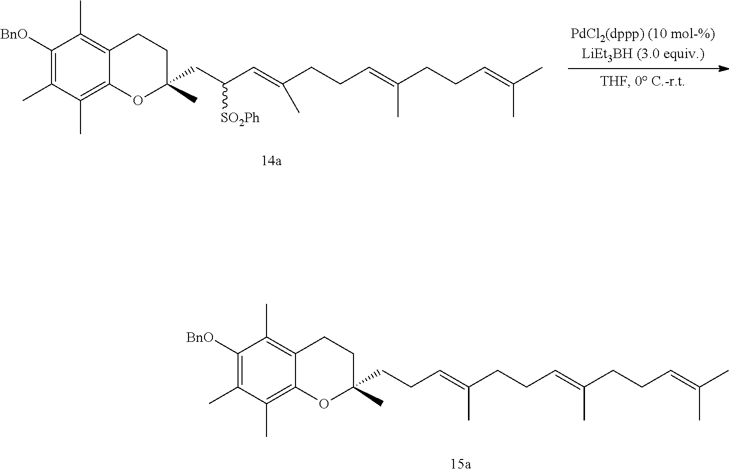

To a mixture of 14a (3.46 g, 5.28 mmol) and bis(diphenylphosphino)ferrocene]palladium(II) dichloride (PdCl₂dppp, 311 mg, 0.528 mmol) under argon was added dry THF (150 mL) and the resulting suspension was cooled to 0° C. Then, lithium triethylborohydride (LiEt₃BH, 1.7 M in THF, 9.3 mL, 15.8 mmol) was added dropwise and the reaction mixture was stirred at 0° C. for 5 hr. and overnight at r.t. The reaction mixture was diluted with Et₂O (500 mL) and successively washed with 1 M aq. sodium cyanide (NaCN, 100 mL), water (100 mL), and brine (100 mL). After drying over anh. Na₂SO₄, the volatile matters were removed under reduced pressure and the crude product was purified by silica gel flash column chromatography using a mixture of petroleum ether and EtOAc (1:0→50:1) as an eluent to give 15a (2.39 g) in 88% yield.

¹H-NMR (CDCl₃, 400 MHZ): δ=7.53-7.47 (m, 2H), 7.43-7.30 (m, 3H), 5.18-5.06 (m, 3H), 4.70 (s, 2H), 2.64-2.57 (m, 2H), 2.22 (s, 3H), 2.19-2.02 (m, 12H), 2.02-1.92 (m, 4H), 1.89-1.73 (m, 2H), 1.70-1.52 (m, 14H), 1.27 ppm (s, 3H).

Step j. Synthesis of (S)-2,5,7,8-tetramethyl-2-((3E,7E)-4,8,12-trimethyltrideca-3,7,11-trien-1-yl)chroman-6-ol (16a)

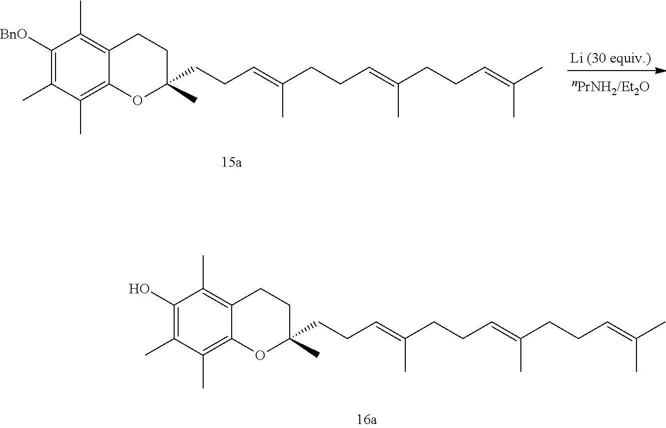

To a cooled (0° C.) suspension of lithium (966 mg, 139 mmol) in n-propylamine (70 mL) and diethyl ether (40 mL) under argon was added a solution of 15a (2.39 g, 4.64 mmol) in Et₂O (30 mL) and the reaction mixture was stirred at r.t. for 3 hr. After re-cooling to 0° C., the reaction mixture was carefully quenched by the addition of sat. aq. NH₄Cl (50 mL) and methanol (50 mL). The organic solvents were removed under reduced pressure at r.t. and the residue was diluted with water (50 mL) and extracted with Et₂O (3×200 mL). The combined organic phases were washed with brine (50 mL), dried over anh. Na₂SO₄ and concentrated under reduced pressure. The crude product was purified by silica gel flash column chromatography using a mixture of petroleum ether and EtOAc (1:0→20:1) as an eluent and re-purified by reversed phase flash chromatography (MeCN/H₂O; 80-100%) to give 16a (1.6 g) in 81% yield.

¹H-NMR (CDCl₃, 400 MHZ): δ=5.17-5.05 (m, 3H), 4.16 (br s, 1H), 2.66-2.56 (m, 2H), 2.18-1.93 (m, 19H), 1.87-1.72 (m, 2H), 1.70-1.49 (m, 14H), 1.25 ppm (s, 3H).

Step t. Synthesis of 2-((S,6E,10E)-3-hydroxy-3,7,11,15-tetramethylhexadeca-6,10,14-trien-1-yl)-3,5,6-trimethylcyclohexa-2,5-diene-1,4-dione (Compound 1-1)

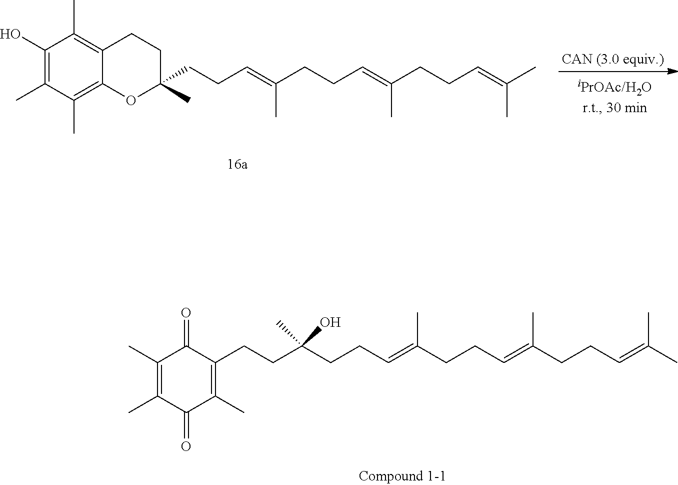

To a solution of 16a (1.6 g, 3.77 mmol) in isopropyl acetate (ⁱPrOAc, 48 mL) at r.t. was added solution of ammonium cerium(IV) nitrate (CAN, 6.19 g, 11.3 mmol) in water (11 mL) and the reaction mixture was stirred at r.t. for 25 min. After cooling to 0° C., the reaction mixture was quenched by the addition of brine (100 mL) and EtOAc (500 mL). The resulting mixture was stirred at r.t. for 5 min. Then, the aqueous phase was separated and the organic phase was washed with brine (50 mL), dried over anh. Na₂SO₄ and concentrated under reduced pressure. The crude product was purified by silica gel flash column chromatography using a mixture of petroleum ether and EtOAc (1:0→40:3) as an eluent and repurified by reversed phase flash chromatography (100% MeCN) to give Compound 1-1 (0.88 g) in 53% yield.

¹H-NMR (CDCl₃, 400 MHZ): δ=5.89-5.80 (m, 1H), 5.19-5.08 (m, 2H), 4.99 (d, ²J_{(H,F)}=47.6 Hz, 2H), 4.86 (d, ²J_{(H,F)}=47.6 Hz, 2H), 2.60-2.49 (m, 2H), 2.33-2.21 (m, 2H), 2.15-1.94 (m, 17H), 1.64 (s, 3H), 1.60 (s, 3H), 1.58-1.45 (m, 4H), 1.25 ppm (s, 3H).

### Example 2: Synthesis of (S,E)-2-(3-hydroxy-3,7,11-trimethyldodeca-6,10-dien-1-yl)-3,5,6-trimethylcyclohexa-2,5-diene-1,4-dione (Compound 3-1)

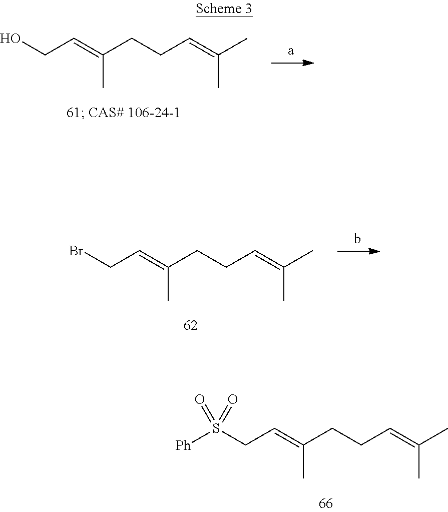 [SMILES: CC(C)=CCC/C(C)=C/CO ; CC(C)=CCC/C(C)=C/CBr ; CC(C)=CCC/C(C)=C/CS(=O)(=O)c1ccccc1]

Step a. Synthesis of (E)-1-bromo-3,7-dimethylocta-2,6-diene (62)

To a cooled (0° C.) solution of 61 (10 g, 64.8 mmol) in dry THF (100 mL) dropwise was added phosphorus tribromide (PBr₃, 7.39 mL, 77.8 mmol) and the reaction mixture was stirred at 0° C. for 40 minutes (min.). The reaction mixture was poured on ice (200 g) and extracted with diethyl ether (Et₂O; 3×200 mL). The combined organic phases were dried over anh. Na₂SO₄ and concentrated under reduced pressure to give 62 (13.4 g) in 95% yield.

Step b. Synthesis of (E)-((3,7-dimethylocta-2,6-dien-1-yl)sulfonyl)benzene (63)

To a cooled (0° C.) solution of 62 (13.4 g, 61.7 mmol) in dry DMF (130 mL) was added phenyl sulfinic acid sodium salt (13.2 g, 80.2 mmol) in one portion and the reaction mixture was stirred at room temperature (r.t.) overnight. The reaction mixture was quenched by the addition of sat. aq. NH₄Cl (200 mL) and EtOAc (1200 mL) and stirred at r.t. for 15 min. The aqueous phase was removed and the organic phase was washed with brine (4×150 mL), dried over anh. Na₂SO₄ and concentrated under reduced pressure. The crude product was purified by silica gel flash column chromatography using a mixture of petroleum ether and EtOAc (1:0→10:1) as an eluent to give 63 (13.4 g) in 78% yield.

¹H-NMR (CDCl₃, 400 MHZ): δ=7.90-7.83 (m, 2H), 7.67-7.59 (m, 1H), 7.57-7.48 (m, 2H), 5.22-5.14 (m, 1H), 5.07-4.98 (m, 1H), 3.80 (d, J=8.0 Hz, 2H), 2.03-1.96 (m, 4H), 1.70-1.65 (m, 3H), 1.59-1.57 (m, 3H), 1.32-1.29 ppm (m, 3H).

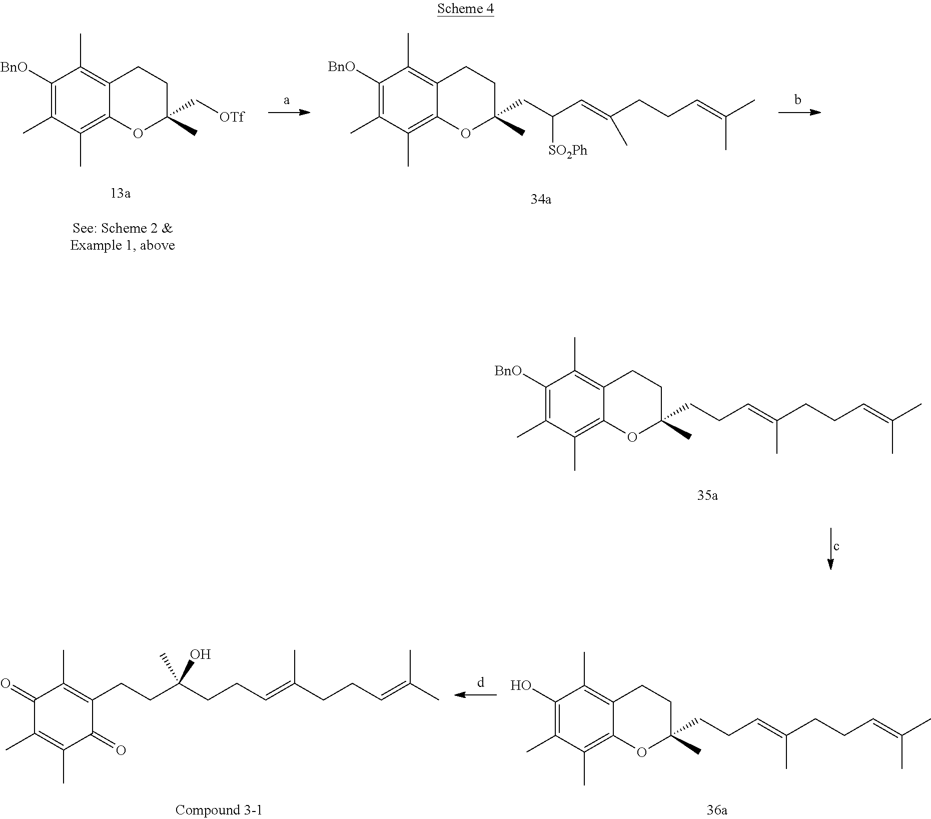

Step a. Synthesis of (2R)-6-(benzyloxy)-2-((E)-4,8-dimethyl-2-(phenylsulfonyl)nona-3,7-dien-1-yl)-2,5,7,8-tetramethylchromane (34a)

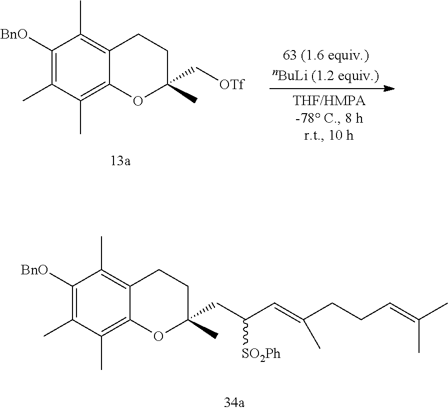

To a solution of 63 (4.25 g, 15.3 mmol; prepared as described in Scheme 3 and the related description, above) in dry THF (60 mL) under argon was added hexamethylphosphoramide (15.9 mmol) and the resulting mixture was cooled to −78° C. Then, ⁿBuLi (2.3 M in hexanes, 5.4 mL, 12.4 mmol) was added dropwise and the reaction mixture was stirred at the same temperature for 30 min. After dropwise addition of 13a (4.38 g, 9.56 mmol; prepared as described in Scheme 2, above) solution in dry THF (60 mL), the reaction mixture was stirred at −78° C. for 8 hours and stirring was continued at r.t. for 20 hours. The reaction mixture was diluted with Et₂O (1000 mL), washed with water (2×150 mL), brine (150 mL), dried over anh. Na₂SO₄ and concentrated under reduced pressure. The crude product was purified by silica gel flash column chromatography using a mixture of petroleum ether and EtOAc (1:0→5:1) as an eluent to give 34a (6.79 g) as a mixture of diastereomers, containing premix of the remaining 63 (about 0.5 equiv.). This crude product was used in the next step without further purification.

Step b. Synthesis of (S,E)-6-(benzyloxy)-2-(4,8-dimethylnona-3,7-dien-1-yl)-2,5,7,8-tetramethylchromane (35a)

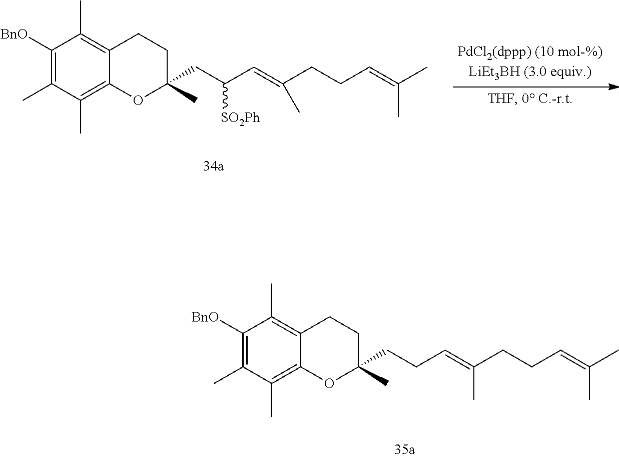

To a mixture of 34a (containing approx. 0.5 equivalents of 63) (6.79 g, approx. 11.5 mmol) and PdCl₂dppp (682 mg, 1.11 mmol) under argon was added dry THF (100 mL) and the resulting suspension was cooled to 0° C. Then, LiEt₃BH (1.7 M in THF, 20.4 ml, 34.7 mmol) was added dropwise and the reaction mixture was stirred at 0° C. for 5 h and overnight at r.t. The reaction mixture was diluted with Et₂O (800 mL) and successively washed with 1 M aq. NaCN (100 mL), water (100 mL), and brine (100 mL). After drying over anh. Na₂SO₄, the volatile matters were removed under reduced pressure and the crude product was purified by silica gel flash column chromatography using a mixture of petroleum ether and EtOAc (1:0→40:1) as an eluent to give 35a (3.9 g).

¹H-NMR (CDCl₃, 400 MHZ): δ=7.52-7.49 (m, 2H), 7.43-7.32 (m, 3H), 5.17-5.07 (m, 2H), 4.70 (s, 2H), 2.63-2.59 (m, 2H), 2.23 (s, 3H), 2.17-2.04 (m, 10H), 2.00-1.96 (m, 2H), 1.89-1.75 (m, 2H), 1.70-1.53 (m, 11H), 1.28 ppm (s, 3H).

Step c. Synthesis of (S,E)-2-(4,8-dimethylnona-3,7-dien-1-yl)-2,5,7,8-tetramethylchroman-6-ol (36a)

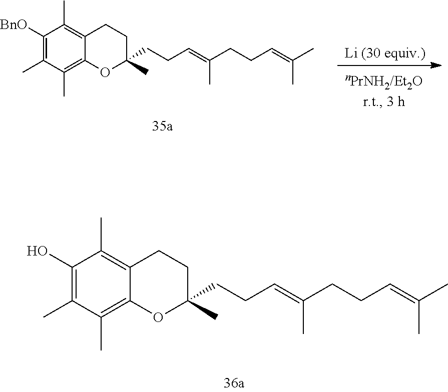

To a cooled (0° C.) suspension of lithium (1.8 g, 260 mmol) in n-propylamine (70 mL) and diethylether (70 mL) under argon was added solution of 35a (3.9 g, 8.71 mmol) in diethylether (30 mL) and the reaction mixture was stirred at r.t. for 3 h. After re-cooling to 0° C., the reaction mixture was carefully quenched by the addition of sat. aq. NH₄Cl (50 mL) and methanol (50 mL). The organic solvents were removed under reduced pressure at r.t. and the residue was diluted with water (100 mL) and extracted with diethylether (3×200 mL). The combined organic phases were washed with brine (100 mL), dried over anh. Na₂SO₄ and concentrated under reduced pressure. The crude product was purified by silica gel flash column chromatography using a mixture of petroleum ether and EtOAc (1:0→40:3) as an eluent to give 36a (2.6 g) in 83% yield.

¹H-NMR (CDCl₃, 400 MHZ): δ=5.16-5.07 (m, 2H), 4.18 (s, 1H), 2.64-2.60 (m, 2H), 2.16-2.03 (m, 13H), 1.99-1.95 (m, 2H), 1.87-1.74 (m, 2H), 1.68-1.51 (m, 11H), 1.26 ppm (s, 3H).

Step d. Synthesis of (S,E)-2-(3-hydroxy-3,7,11-trimethyldodeca-6,10-dien-1-yl)-3,5,6-trimethylcyclohexa-2,5-diene-1,4-dione (Compound 3-1)

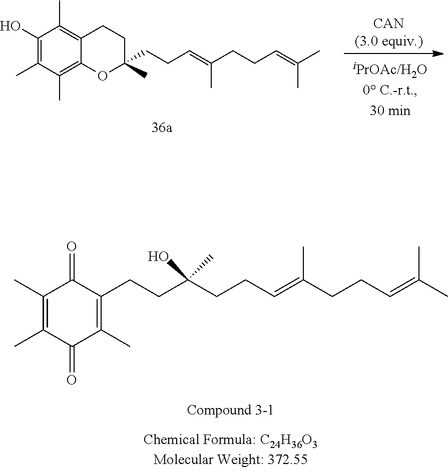

To a cooled (ice bath) solution of 36a (300 mg, 0.56 mmol) in ⁱPrOAc (7 mL) was added solution of ammonium cerium(IV) nitrate (1380 mg, 1.68 mmol) in water (1.8 mL) and the reaction mixture was stirred at r.t. for 25 min. After cooling to 0° C., the reaction mixture was quenched by the addition of brine (60 mL) and EtOAc (300 mL). The resulting mixture was stirred at r.t. for 5 min. Then, aqueous phase was separated and the organic phase was washed with water (80 mL) and brine (80 mL), dried over anh. Na₂SO₄ and concentrated under reduced pressure. The crude product was purified by silica gel flash column chromatography using a mixture of petroleum ether and EtOAc (1:0→10:1) as an eluent and repurified by reversed phase flash chromatography (100% MeCN) to give Compound 3-1 (236 mg) in 75% yield.

¹H-NMR (CDCl₃, 400 MHZ): δ=5.17-5.06 (m, 2H), 2.57-2.53 (m, 2H), 2.13-1.97 (m, 15H), 1.71-1.50 (m, 14H), 1.25 ppm (s, 3H).

### Example 3: Synthesis of (S)-2-(3-hydroxy-3,7-dimethyloct-6-en-1-yl)-3,5,6-trimethylcyclohexa-2,5-diene-1,4-dione (Compound 5-1)

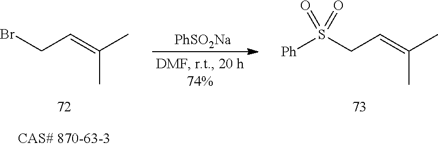

To a cooled (0° C.) solution of 72 (20 g, 134 mmol) in dry DMF (200 mL) was added phenyl sulfinic acid sodium salt (28.6 g, 174 mmol) in one portion and the reaction mixture was stirred at r.t. overnight. Most of the solvent was removed under reduced pressure and the residue was quenched by the addition of sat. aq. NH₄Cl (200 mL) and EtOAc (1200 mL) and stirred at r.t. for 15 min. The aqueous phase was removed and the organic phase was washed with water (4×150 mL) and brine (100 mL), dried over anh. Na₂SO₄ and concentrated under reduced pressure. The crude product was purified by silica gel flash column chromatography using a mixture of petroleum ether and EtOAc (1:0→20:3) as an eluent to give 73 (20.9 g) in 74% yield.

¹H-NMR (CDCl₃, 400 MHz): δ=7.90-7.82 (m, 2H), 7.67-7.59 (m, 1H), 7.57-7.49 (m, 2H), 5.22-5.14 (m, 1H), 3.81-3.75 (m, 2H), 1.73-1.68 (m, 3H), 1.32-1.29 ppm (m, 3H).

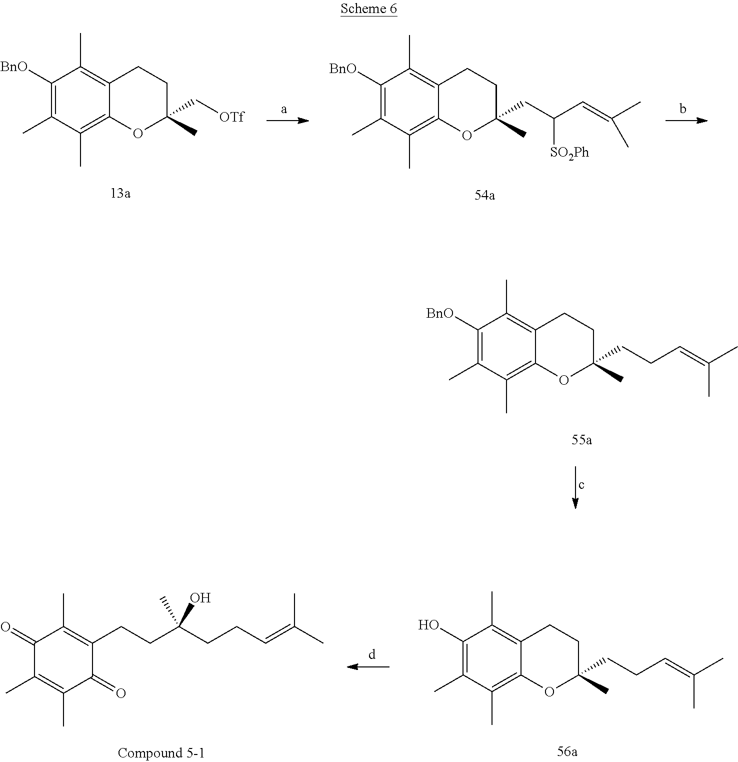

Step a. Synthesis of (2R)-6-(benzyloxy)-2,5,7,8-tetramethyl-2-(4-methyl-2-(phenylsulfonyl)pent-3-en-1-yl)chromane (54a)

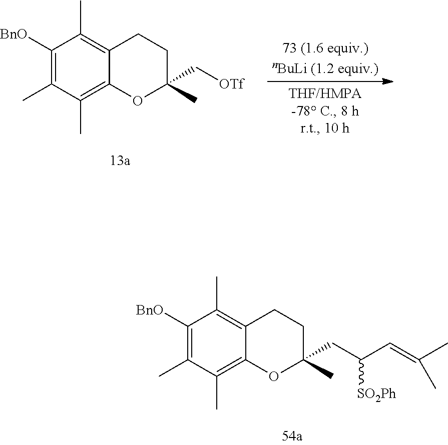

To a solution of 73 (3.21 g, 15.3 mmol; prepared as described in Scheme 5 and the related description) in dry THF (60 mL) under argon was added hexamethylphosphoramide (15.9 mmol) and the resulting mixture was cooled to −78° C. Then, ⁿBuLi (2.3 M in hexanes, 5.4 ml, 12.4 mmol) was added dropwise and the reaction mixture was stirred at the same temperature for 30 min. After dropwise addition of 13a (4.38 g, 9.56 mmol) solution in dry THF (60 mL), the reaction mixture was stirred at −78° C. for 8 h and stirring was continued at r.t. for 20 h. The reaction mixture was diluted with Et₂O (800 mL), washed with water (2×150 mL) and brine (150 mL), dried over anh. Na₂SO₄ and concentrated under reduced pressure. The crude product was purified by silica gel flash column chromatography using a mixture of petroleum ether and EtOAc (1:0→5:1) as an eluent to give 54a (6 g) as a mixture of diastereomers, containing premix of the remaining 73 (about 1.3 equiv.). This material was used in the next step without further purification.

Step b. Synthesis of (S)-6-(benzyloxy)-2,5,7,8-tetramethyl-2-(4-methylpent-3-en-1-yl)chromane (55a)

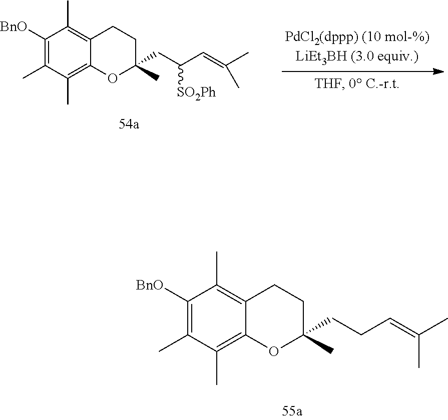

To a mixture of 54a (containing approx. 1.3 equivalents of 73) (6 g, approx. 11.6 mmol) and PdCl₂dppp (680 mg, 1.15 mmol) under argon was added dry THF (100 mL) and the resulting suspension was cooled to 0° C. Then, LiEt₃BH (1.7 M in THF, 20.4 mL, 34.7 mmol) was added dropwise and the reaction mixture was stirred at 0° C. for 5 h. and overnight at r.t. The reaction mixture was diluted with Et₂O (800 mL) and successively washed with 1 M aq. NaCN (100 mL), water (100 mL), and brine (100 mL). After drying over anh. Na₂SO₄, the volatile matters were removed under reduced pressure and the crude product was purified by silica gel flash column chromatography using a mixture of petroleum ether and EtOAc (1:0→40:1) as an eluent to give 55a (2.2 g).

¹H-NMR (CDCl₃, 400 MHZ): δ=7.52-7.50 (m, 2H), 7.43-7.39 (m, 2H), 7.36-7.32 (m, 1H), 5.16-5.12 (m, 1H), 4.70 (s, 2H), 2.63-2.59 (m, 2H), 2.23 (s, 3H), 2.17-2.10 (m, 8H), 1.89-1.75 (m, 2H), 1.69-1.53 (m, 8H), 1.27 ppm (s, 3H).

Step c. Synthesis of (S)-2,5,7,8-tetramethyl-2-(4-methylpent-3-en-1-yl)chroman-6-ol (56a)

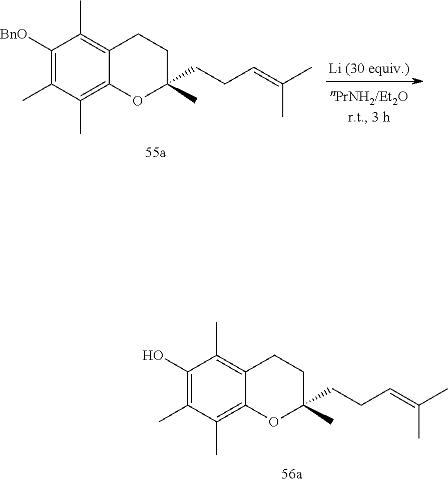 [SMILES: CC(C)=CCC[C@@]1(C)CCc2c(C)c(OCc3ccccc3)c(C)c(C)c2O1 ; CC(C)=CCC[C@@]1(C)CCc2c(C)c(O)c(C)c(C)c2O1]

To a cooled (0° C.) suspension of lithium (1.2 g, 174 mmol) in n-propylamine (50 mL) and diethylether (20 mL) under argon was added solution of 55a (2.2 g, 5.8 mmol) in diethylether (30 mL) and the reaction mixture was stirred at r.t. for 3 h. After re-cooling to 0° C., the reaction mixture was carefully quenched by the addition of sat. aq. NH₄Cl (30 mL) and methanol (30 mL). The organic solvents were removed under reduced pressure at r.t. and the residue was diluted with water (100 mL) and extracted with diethylether (3×200 mL). The combined organic phases were washed with brine (100 mL), dried over anh. Na₂SO₄ and concentrated under reduced pressure. The crude product was purified by silica gel flash column chromatography using a mixture of petroleum ether and EtOAc (1:0→40:3) as an eluent to give 56a (1.55 g) in 92% yield.

¹H-NMR (CDCl₃, 400 MHZ): δ=5.14-5.10 (m, 1H), 4.17 (s, 1H), 2.63-2.60 (m, 2H), 2.16-2.09 (m, 11H), 1.87-1.73 (m, 2H), 1.68-1.50 (m, 8H), 1.25 ppm (s, 3H).

Step d. Synthesis of (S)-2-(3-hydroxy-3,7-dimethyloct-6-en-1-yl)-3,5,6-trimethylcyclohexa-2,5-diene-1,4-dione (Compound 5-1)

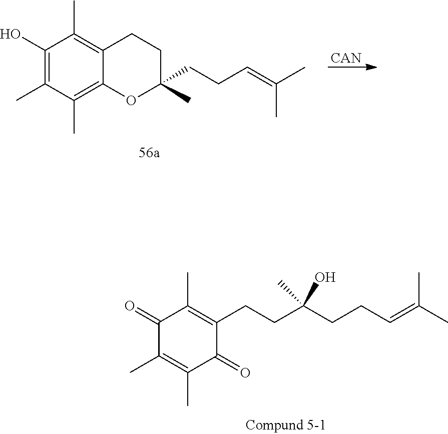

To a cooled (ice bath) solution of 56a (300 mg, 1.04 mmol) in ⁱPrOAc (15 mL) was added solution of ammonium cerium(IV) nitrate (1.7 g, 3.12 mmol) in water (3 mL) and the reaction mixture was stirred at r.t. for 25 min. After cooling to 0° C., the reaction mixture was quenched by the addition of brine (60 mL) and EtOAc (350 mL). The resulting mixture was stirred at r.t. for 5 min. Then, the aqueous phase was separated and the organic phase was washed with water (60 mL) and brine (60 mL), dried over anh. Na₂SO₄ and concentrated under reduced pressure. The crude product was purified by silica gel flash column chromatography using a mixture of petroleum ether and EtOAc (1:0→10:1) as an eluent to give Compound 5-1 (268 mg) in 84% yield.

¹H-NMR (CDCl₃, 400 MHZ): δ32 5.16-5.12 (m, 1H), 2.57-2.53 (m, 2H), 2.11-2.01 (m, 11H), 1.69 (s, 3H), 1.64 (s, 3H), 1.56-1.48 (m, 5H), 1.25 ppm (s, 3H).

### Example 4: Synthesis of (R,E)-2-(3-hydroxy-3,7,11-trimethyldodeca-6,10-dien-1-yl)-3,5,6-trimethylcyclohexa-2,5-diene-1,4-dione (Compound 2-1)

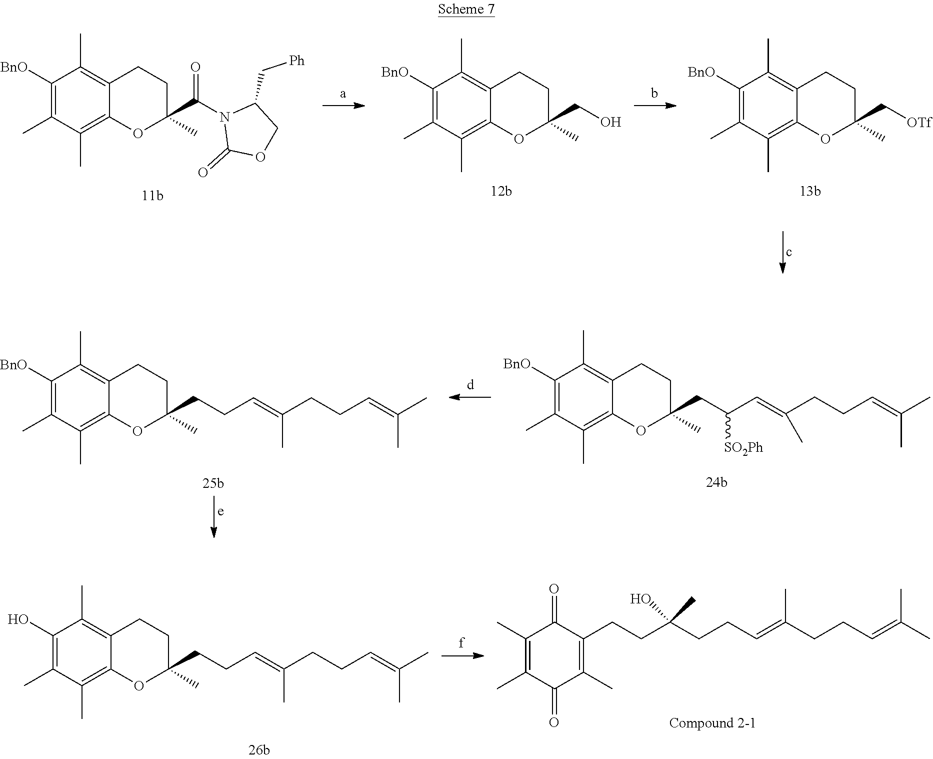

Step a. Synthesis of (S)-(6-(benzyloxy)-2,5,7,8-tetramethylchroman-2-yl)methanol (12b)

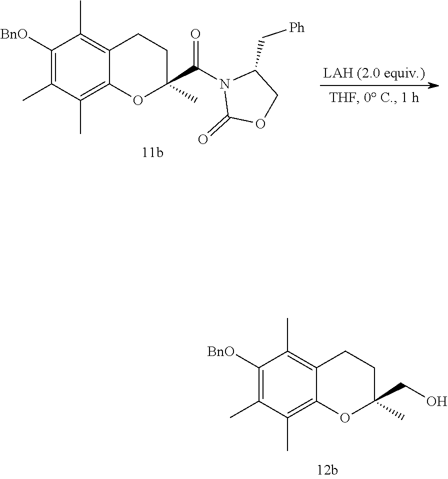 [SMILES: Cc1c(C)c2c(c(C)c1OCc1ccccc1)CC[C@@](C)(C(=O)N1C(=O)OC[C@H]1Cc1ccccc1)O2 ; Cc1c(C)c2c(c(C)c1OCc1ccccc1)CC[C@@](C)(CO)O2]

Compound 11b was prepared according to Scheme 2, above and converted to 12b using lithium aluminum hydride in THE essentially as described above in Scheme 2, step. f.

Step b. Synthesis of (S)-(6-(benzyloxy)-2,5,7,8-tetramethylchroman-2-yl)methyl Trifluoromethanesulfonate (13b)

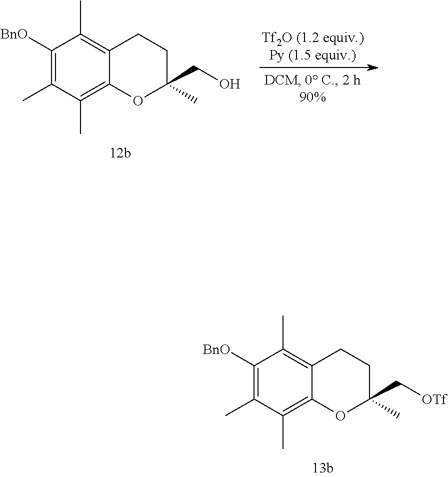 [SMILES: Cc1c(C)c2c(c(C)c1OCc1ccccc1)CC[C@@](C)(CO)O2 ; Cc1c(C)c2c(c(C)c1OCc1ccccc1)CC[C@@](C)(COS(=O)(=O)C(F)(F)F)O2]

To a solution of 12b (8.6 g, 26.3 mmol) in dry DCM (150 mL) was added pyridine (3.2 mL, 39.5 mmol). After cooling to 0° C., trifluorometanesulfonic anhydride (5.3 mL, 31.6 mmol) was added dropwise and the reaction mixture was stirred at 0° C. for 2 h. The reaction mixture was quenched with ice (30 g) and allowed to warm to r.t. Then, EtOAc (600 mL) and water (150 mL) were added and the resulting mixture was stirred at r.t. for 15 min. The aqueous phase was separated and the organic phase was washed with brine (2×150 ml) and dried over anh. Na₂SO₄. After removal of volatile matters, 13b (10.9 g) was obtained in 90% yield.

¹H-NMR (CDCl₃, 400 MHZ): δ=7.55-7.30 (m, 5H), 4.70 (s, 2H), 4.53-4.40 (m, 2H), 2.72-2.61 (m, 2H), 2.23 (s, 3H), 2.18 (s, 3H), 2.10 (s, 3H), 2.04-1.93 (m, 1H), 1.92-1.80 (m, 1H), 1.38 ppm (s, 3H).

Step c. Synthesis of (2S)-6-(benzyloxy)-2-((E)-4,8-dimethyl-2-(phenylsulfonyl)nona-3,7-dien-1-yl)-2,5,7,8-tetramethylchromane (24b)

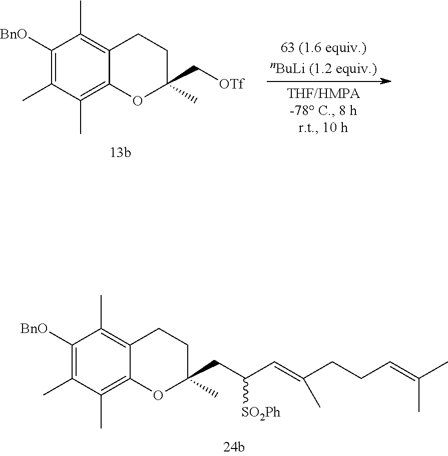

To a solution of 63 (4.84 g, 17.4 mmol; prepared as described above) in dry THF (60 mL) under argon was added hexamethylphosphoramide (15.9 mmol) and the resulting mixture was cooled to −78° C. Then, ⁿBuLi (2.3 M in hexanes, 5.7 mL, 13.1 mmol) was added dropwise and the reaction mixture was stirred at the same temperature for 30 min. After dropwise addition of 13b (5.00 g, 10.9 mmol) solution in dry THF (60 mL), the reaction mixture was stirred at −78° C. for 8 h. and stirring was continued at r.t. for 20 h. The reaction mixture was diluted with Et₂O (1000 mL), washed with water (2×150 mL), brine (150 mL), dried over anh. Na₂SO₄ and concentrated under reduced pressure. The crude product was purified by silica gel flash column chromatography using a mixture of petroleum ether and EtOAc (1:0→5:1) as an eluent to give 24b (7.73 g) as a mixture of diastereomers, containing premix of the remaining 63 (about 0.5 equiv.). This crude product was used in the next step without further purification.

Step d. Synthesis of (R,E)-6-(benzyloxy)-2-(4,8-dimethylnona-3,7-dien-1-yl)-2,5,7,8-tetramethylchromane (25b)

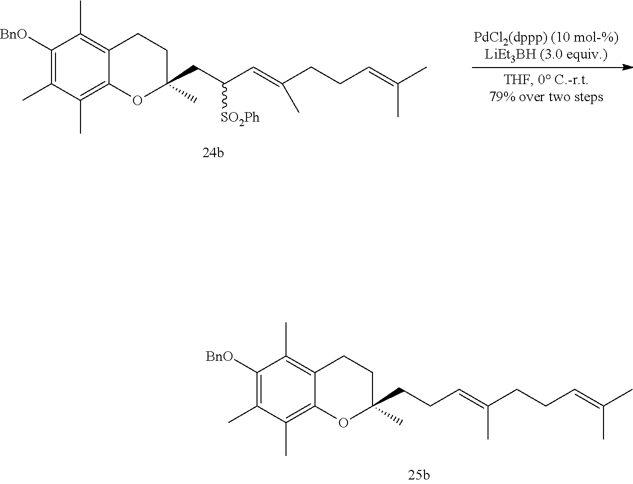

To a mixture of 24b (containing approx. 0.5 equivalents of 63) (7.73 g, approx. 13.2 mmol) and PdCl₂dppp (779 mg, 1.32 mmol) under argon was added dry THF (100 mL) and the resulting suspension was cooled to 0° C. Then, LiEt₃BH (1.7 M in THF, 23.3 mL, 39.6 mmol) was added dropwise and the reaction mixture was stirred at 0° C. for 5 h. and overnight at r.t. The reaction mixture was diluted with Et₂O (800 mL) and successively washed with 1 M aq. NaCN (100 mL), water (100 mL), and brine (100 mL). After drying over anh. Na₂SO₄, the volatile matters were removed under reduced pressure and the crude product was purified by silica gel flash column chromatography using a mixture of petroleum ether and EtOAc (1:0→40:1) as an eluent to give 25b (3.83 g) in 79% yield over two steps.

¹H-NMR (CDCl₃, 400 MHZ): δ=7.56-7.50 (m, 2H), 7.46-7.29 (m, 3H), 5.21-5.08 (m, 2H), 4.73 (s, 2H), 2.67-2.59 (m, 2H), 2.25 (s, 3H), 2.23-2.05 (m, 10H), 2.05-1.96 (m, 2H), 1.93-1.76 (m, 2H), 1.75-1.54 (m, 11H), 1.30 ppm (s, 3H).

Step e. Synthesis of (R,E)-2-(4,8-dimethylnona-3,7-dien-1-yl)-2,5,7,8-tetramethylchroman-6-ol (26b)

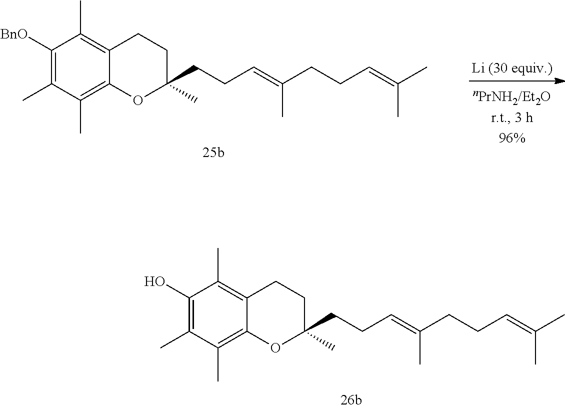 [SMILES: CC(C)=CCC/C(C)=C/CC[C@]1(C)CCc2c(C)c(OCc3ccccc3)c(C)c(C)c2O1 ; CC(C)=CCC/C(C)=C/CC[C@]1(C)CCc2c(C)c(O)c(C)c(C)c2O1]

To a cooled (0° C.) suspension of lithium (1.77 g, 255 mmol) in n-propylamine (100 mL) and diethylether (60 mL) under argon was added a solution of 25b (3.8 g, 8.51 mmol) in diethylether (40 mL) and the reaction mixture was stirred at r.t. for 3 h. After re-cooling to 0° C., the reaction mixture was carefully quenched by the addition of sat. aq. NH₄Cl (50 mL) and methanol (50 mL). The organic solvents were removed under reduced pressure at r.t. and the residue was diluted with water (100 mL) and extracted with diethylether (3×200 mL). The combined organic phases were washed with brine (100 mL), dried over anh. Na₂SO₄ and concentrated under reduced pressure. The crude product was purified by silica gel flash column chromatography using a mixture of petroleum ether and EtOAc (1:0→40:3) as an eluent to give 25b (2.92 g) in 96% yield.

¹H-NMR (CDCl₃, 400 MHZ): δ=5.18-5.06 (m, 2H), 4.19 (s, 1H), 2.67-2.58 (m, 2H), 2.21-2.02 (m, 13H), 2.01-1.94 (m, 2H), 1.90-1.73 (m, 2H), 1.73-1.49 (m, 11H), 1.26 ppm (s, 2H).

Step f. Synthesis of (R,E)-2-(3-hydroxy-3,7,11-trimethyldodeca-6,10-dien-1-yl)-3,5,6-trimethylcyclohexa-2,5-diene-1,4-dione (Compound 2-1)

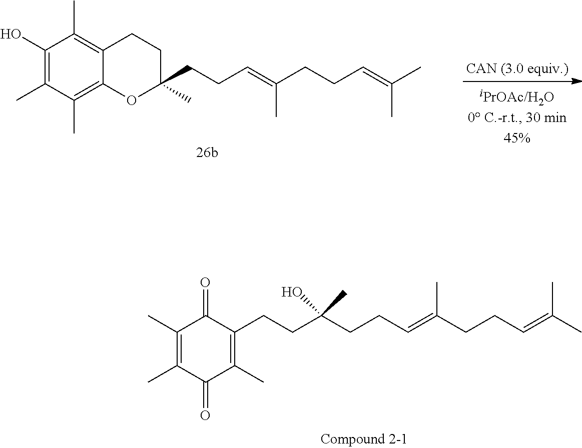

To a cooled (ice bath) solution of 26b (216 mg, 0.606 mmol) in ⁱPrOAc (7.5 mL) was added solution of ammonium cerium(IV) nitrate (998 mg, 1.82 mmol) in water (1.8 mL) and the reaction mixture was stirred at r.t. for 25 min. After cooling to 0° C., the reaction mixture was quenched by the addition of brine (60 mL) and EtOAc (300 mL). The resulting mixture was stirred at r.t. for 5 min. Then, aqueous phase was separated and the organic phase was washed with water (80 mL) and brine (80 mL), dried over anh. Na₂SO₄ and concentrated under reduced pressure. The crude product was purified by silica gel flash column chromatography using a mixture of petroleum ether and EtOAc (1:0→10:1) as an eluent and repurified by reversed phase flash chromatography (100% MeCN) to give Compound 2-1 (101 mg) in 45% yield.

¹H-NMR (CDCl₃, 400 MHZ): δ=5.20-5.04 (m, 2H), 2.60-2.50 (m, 2H), 2.21-1.92 (m, 15H), 1.73-1.44 (m, 13H), 1.25 ppm (s, 3H).

### Example 5: Synthesis of (R)-2-(3-hydroxy-3,7-dimethyloct-6-en-1-yl)-3,5,6-trimethylcyclohexa-2,5-diene-1,4-dione (Compound 4-1)

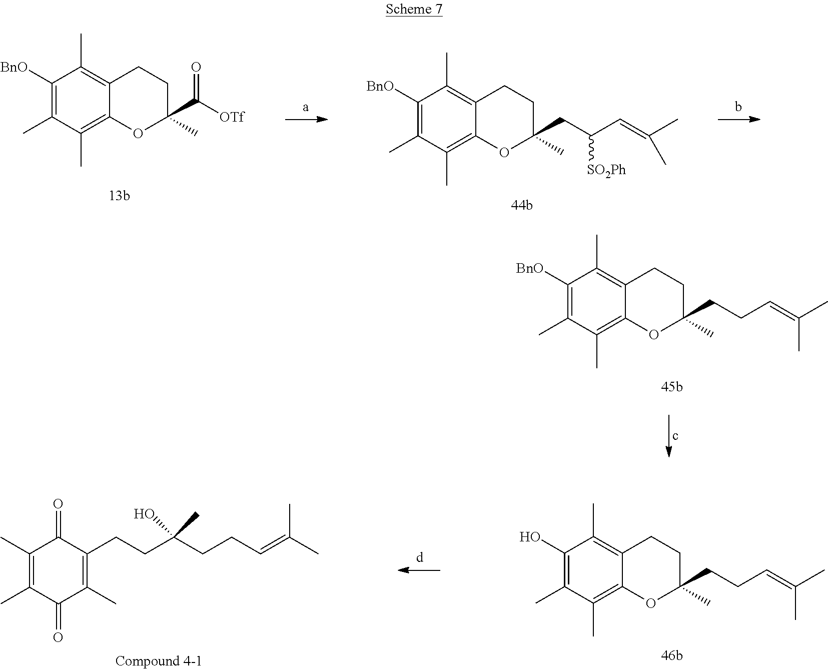

Step a. Synthesis of (2S)-6-(benzyloxy)-2,5,7,8-tetramethyl-2-(4-methyl-2-(phenylsulfonyl)pent-3-en-1-yl)chromane (44b)

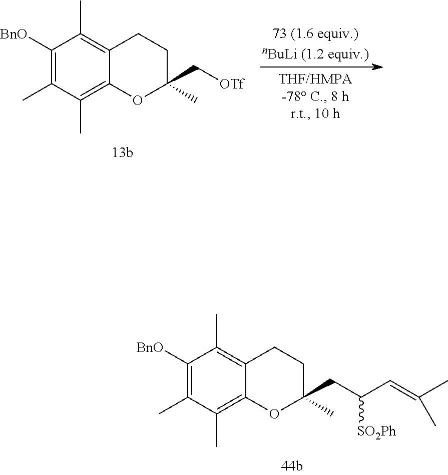

To a solution of 73 (3.66 g, 17.4 mmol; prepared as described above) in dry THF (60 mL) under argon was added hexamethylphosphoramide (15.9 mmol) and the resulting mixture was cooled to −78° C. Then, ⁿBuLi (2.3 M in hexanes, 5.7 mL, 13.1 mmol) was added dropwise and the reaction mixture was stirred at the same temperature for 30 min. After dropwise addition of 13b (5.00 g, 10.9 mmol) solution in dry THF (60 mL), the reaction mixture was stirred at −78° C. for 8 h. and stirring was continued at r.t. for 20 h. The reaction mixture was diluted with Et₂O (1000 mL), washed with water (2×150 mL), brine (150 mL), dried over anh. Na₂SO₄ and concentrated under reduced pressure. The crude product was purified by silica gel flash column chromatography using a mixture of petroleum ether and EtOAc (1:0→5:1) as an eluent to give 44b (4.95 g) as a mixture of diastereomers, containing premix of the remaining 73 (about 1.3 equiv.). This crude product was used in the next step without further purification.

Step b. Synthesis of (R)-6-(benzyloxy)-2,5,7,8-tetramethyl-2-(4-methylpent-3-en-1-yl)chromane (45b)

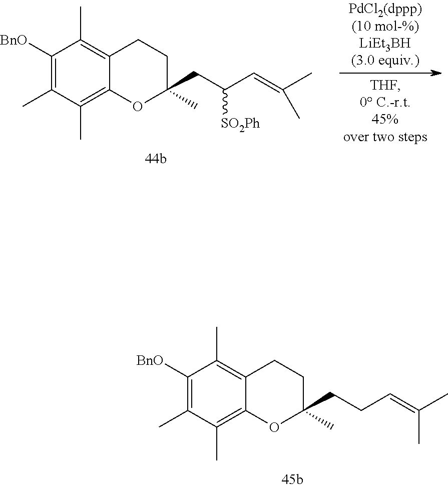

To a mixture of 44b (containing approx. 1.3 equivalents of 73) (4.95 g, approx. 14.5 mmol) and PdCl₂dppp (855 mg, 1.45 mmol) under argon was added dry THF (100 mL) and the resulting suspension was cooled to 0° C. Then, LiEt₃BH (1.7 M in THF, 25.6 mL, 43.5 mmol) was added dropwise and the reaction mixture was stirred at 0° C. for 5 h. and overnight at r.t. The reaction mixture was diluted with Et₂O (800 mL) and successively washed with 1 M aq. NaCN (100 mL), water (100 mL), and brine (100 mL). After drying over anh. Na₂SO₄, the volatile matters were removed under reduced pressure and the crude product was purified by silica gel flash column chromatography using a mixture of petroleum ether and EtOAc (1:0→40:1) as an eluent to give 45b (1.87 g) in 45% yield over two steps.

¹H-NMR (CDCl₃, 400 MHZ): δ=7.55-7.50 (m, 2H), 7.45-7.39 (m, 2H), 7.39-7.32 (m, 1H), 5.19-5.12 (m, 1H), 4.72 (s, 2H), 2.66-2.58 (m, 2H), 2.24 (s, 3H), 2.21-2.10 (m, 8H), 1.93-1.75 (m, 2H), 1.73-1.53 (m, 8H), 1.29 ppm (s, 3H).

Step c. Synthesis of (R)-2,5,7,8-tetramethyl-2-(4-methylpent-3-en-1-yl)chroman-6-ol (46b)

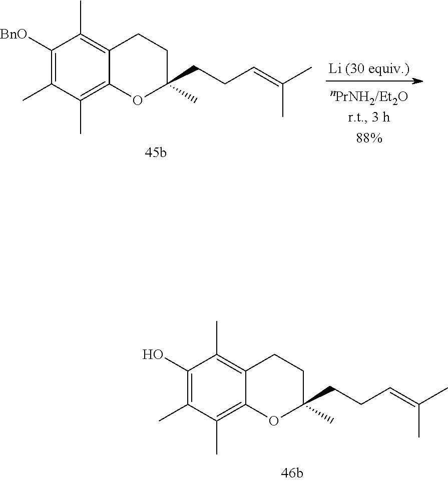 [SMILES: CC(C)=CCC[C@]1(C)CCc2c(C)c(OCc3ccccc3)c(C)c(C)c2O1 ; CC(C)=CCC[C@]1(C)CCc2c(C)c(O)c(C)c(C)c2O1]

To a cooled (0° C.) suspension of lithium (1.03 g, 148 mmol) in n-propylamine (50 mL) and diethylether (20 mL) under argon was added solution of 45b (1.87 g, 4.94 mmol) in diethylether (30 mL) and the reaction mixture was stirred at r.t. for 3 h. After re-cooling to 0° C., the reaction mixture was carefully quenched by the addition of sat. aq. NH₄Cl (30 mL) and methanol (30 mL). The organic solvents were removed under reduced pressure at r.t. and the residue was diluted with water (100 mL) and extracted with diethylether (3×200 mL). The combined organic phases were washed with brine (100 mL), dried over anh. Na₂SO₄ and concentrated under reduced pressure. The crude product was purified by silica gel flash column chromatography using a mixture of petroleum ether and EtOAc (1:0→40:3) as an eluent to give 46b (1.25 g) in 88% yield.

¹H-NMR (CDCl₃, 400 MHZ): δ=5.18-5.10 (m, 1H), 4.21 (s, 1H), 2.68-2.58 (m, 2H), 2.22-2.07 (m, 11H), 1.90-1.73 (m, 2H), 1.72-1.50 (m, 8H), 1.26 ppm (s, 3H).

Step d. Synthesis of (R)-2-(3-hydroxy-3,7-dimethyloct-6-en-1-yl)-3,5,6-trimethylcyclohexa-2,5-diene-1,4-dione (Compound 4-1)

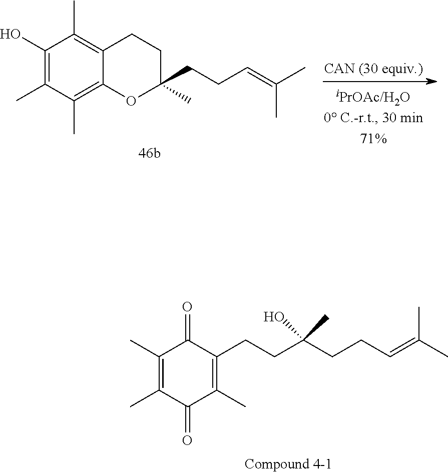

To a cooled (ice bath) solution of 46b (148 mg, 0.513 mmol) in ⁱPrOAc (6.4 mL) was added solution of ammonium cerium(IV) nitrate (844 mg, 1.54 mmol) in water (1.5 mL) and the reaction mixture was stirred at r.t. for 25 min. After cooling to 0° C., the reaction mixture was quenched by the addition of brine (30 mL) and EtOAc (150 mL). The resulting mixture was stirred at r.t. for 5 min. Then, the aqueous phase was separated and the organic phase was washed with water (30 mL) and brine (30 mL), dried over anh. Na₂SO₄ and concentrated under reduced pressure. The crude product was purified by silica gel flash column chromatography using a mixture of petroleum ether and EtOAc (1:0→10:1) as an eluent and repurified by reversed phase flash chromatography (100% MeCN) to give Compound 4-1 (110 mg) in 71% yield.

¹H-NMR (CDCl₃, 400 MHz): δ=5.17-5.10 (m, 1H), 2.59-2.51 (m, 2H), 2.13-1.98 (m, 11H), 1.72-1.67 (m, 3H), 1.66-1.62 (m, 3H), 1.59-1.46 (m, 4H), 1.25 ppm (s, 3H).

### Example 6: Frataxin Deficient Fibroblast Viability Assay (the “BSO Assay”)

Reference: Matthias L. Jauslin, Thomas Wirth, Thomas Meier and Fabrice Schoumacher, A cellular model for Friederichs Ataxia reveals small-molecule glutathione peroxidase mimetics as novel treatment strategy, Human Molecular Genetics, 2002, Vol. 11 (24): 3055-3063.

This Example was used to evaluate the various new compositions for their potential efficacy in the treatment of Friedreich's Ataxia (FA or FRDA) based on their potential to inhibit ferroptosis based on this BSO assay—performed substantially as described in the above cited Reference (Matthias et al.). This data can be used to select candidates for further testing, including animal studies directed to development of active therapeutic agents.

### Introduction

The assay utilizes frataxin deficient fibroblasts (i.e., fibroblasts from Friedreich's ataxia (FRDA) patient material) as a means to assay cell viability by determining how compounds of interest can potentially inhibit/delay/prevent L-buthionine-sulfoximine-induced (BSO-induced) cell death in diseased and control (healthy) cells.

### Test Articles

The stock solutions of the test articles (and control compounds) were prepared in dimethyl sulfoxide (DMSO at 10 mM). Working stock solutions (2 times concentrated) were prepared on the day of the experiment in respective cell culture medium to be used for assay (detailed medium description below). A listing of compounds used as test articles in this Example 12 are found in Table 1, below.

### Assay Experimental

A patient derived frataxin deficient fibroblast cell line was obtained from Coriell Institute. More specifically, the following cell lines/DNA samples were obtained from the NIGMS Human Genetic Cell Repository at the Coriell Institute for Medical Research: GM03665. To evaluate importance of various growth conditions on cell susceptibility to BSO toxicity cells were grown on:

MEM (Sigma-Aldrich) 15% Fetal Bovine Serum (FBS) without growth factors;

MEM (Sigma-Aldrich) 15% FBS with growth factors (Catalogue number 100-18B, Recombinant Human FGF-basic (154 a.a.) and catalogue number AF-100-15, Animal-Free Recombinant Human EGF from Peprotech);

MEM199/MEM EBS (Bioconcept Ltd.) 10% FBS, insulin 10 μg/ml, L-glutamine 2 mM with growth factors (Catalogue number 100-18B, Recombinant Human FGF-basic (154 a.a.) and catalogue number AF-100-15, Animal-Free Recombinant Human EGF from Peprotech).

To conduct an experiment, fibroblast cells (from all growth conditions) were seeded (100 μL (cells 3×10∧3/well) on 96-well plates in MEM199/MEM EBS medium 10% FBS, insulin 10 μg/mL, L-glutamine 2 mM with growth factors and allowed to grow on plate for 24 hrs. After 24 hrs., media was removed and then test compounds of interest were added (100 μL, 2 times concentrated stock) to 96-well plates (end DMSO concentration not exceeding 0.5%) and incubated for 24 hours. Then L-buthionine-sulfoximine (BSO, from Acros Organics, Cat. No. 235520010) was added (100 μL, 2 times concentrated stock) at end concentrations ranging from 1 to 10 mM. Both test compounds and BSO were dissolved in MEM199/MEM EBS medium 10% FBS, insulin 10 μg/mL, L-glutamine 2 mM with growth factors. Cell viability was monitored and either after 24- or 48-hours cell viability was assayed by MTT (Thiazolyl blue tetrazolium bromide) assay. For the MTT assay, media was removed and 100 μL of MTT 1 mg/mL was added, incubated for 2 hours at +37° C., then medium was removed and 100 μL of isopropanol added to dissolve sediment. Absorption was measured at 570 (OD570) and 650 (OD650) nm wavelengths. Control cells (vehicle instead of BSO and compound) and vehicle control (with BSO, but vehicle instead of test compound) were treated in the same manner as cells with BSO and compound. All cell media contained penicillin and streptomycin 100 U/mL each.

### Calculations

Absorption readouts were processed as follows: Value_{Δ}=Value_{OD570}-Value_{OD650} and the obtained values were used to calculate cell viability as % of control cell (i.e., no BSO, no compound, just vehicle) viability.

### Replicates

Except as otherwise indicated in Table 1, all samples were run in at least 3 replicates for test compounds and at least 8 replicates for controls (i.e., both control (no BSO, no compound) and vehicle control (i.e., with BSO, no compound). Exact N per data points are indicated in the figures/table legends. Results for the compounds (test articles and controls) tested are listed in Table 1, below.

### Discussion

This is a cell-based assay measuring cytotoxicity which results from oxidative stress subsequent to depletion of endogenous glutathione defense mechanisms. The cells were primary Friedreich Ataxia (FA) patient fibroblasts which were incubated for 48 hrs. in BSO, an inhibitor of gamma glutamylsynthetase, an enzyme required for glutathione production. Relative to healthy control fibroblasts, FA patient fibroblasts are more susceptible to the BSO induced cell death due to the loss of frataxin and subsequent accumulation of cytosolic iron, which accelerates the process of ROS driven lipid peroxidation. In the assay, cells were pretreated with drug at decreasing doses from 250 nM down to 6.125 nM the day before the BSO was added at a fixed dose of 10 mM. Cytotoxicity was measured with MTT assay 48 hrs. later and reported as the percent of MTT absorbance normalized to cells grown in media without BSO for 48 hrs. Unless otherwise indicated, each data point was performed in triplicate wells of a 96 well plate. Data obtained is presented below in Table 1.

In summary, the data in Table 1 for this assay suggests that Compounds 1-1, 3-1 and 5-1 may be as good as vatiquinone in addressing a disease, disorder or condition associated with ferroptosis. It is noteworthy that each of Compounds 1-1, 3-1 and 5-1 are S-enantiomers (as compared with vatiquinone, Compound 2-1 and 4-1—which possesses the R-configuration). The data also suggests that while 2-1 and 4-1 appear to be not quite as effective as vatiquinone, they are still somewhat potent. Indeed, this assay suggests that vatiquinone and all of Compounds 1-1, 2-1, 3-1, 4-1 and 5-1 are protective against ferroptosis.

### Example 7: Study of Concentration of Therapeutic Agents in Blood and Brain Tissue in Rat Following Oral Administration

### Purpose

This study was intended to examine and compare the concentration of the various therapeutic agents tested in blood and brain tissue shortly after oral administration to rats.

### Protocol

The experimental procedures were carried out in accordance with the guidelines of the European Community and local laws and policies and were approved by the Latvian Animal Protection Ethical Committee, Food and Veterinary Service, Riga, Latvia. Animals were housed in the animal facilities at the Latvian Institute of Organic Synthesis (LIOS) under standard conditions (21-23° C., 12 h light-dark cycle) with unlimited access to food (R70 diet, Lantmännen Lantbruk, Sweden) and water. Male Sprague Dawley (SD) rats (8 weeks old at the time of purchase, from Envigo) were used for the study and were allowed an acclimatization period of at least 1 week prior to treatment. No drugs or vaccines were administered before the start of the study. Only healthy animals were included in the study. Animals were weighed on the day of treatment (before treatment) to calculate the required volume of working solution of compound (i.e. therapeutic agent) administered. No drugs except the therapeutic agent to be tested were administered during the study. Rats were dosed orally with compound (1 times weight μL, solution 10 mg/mL, dissolved in sesame oil).

Animals were anaesthetized using 5% isoflurane inhalation at selected time points after administration of test compounds. Then chest was opened and blood drawn from the heart and collected in EDTA coated microtubes (Sarstedt). Transcardial perfusion was performed afterwards by perfusing ice-cold PBS solution for 4 minutes, ˜55 mL total perfusion volume. Afterwards brain tissues were collected in microtubes and frozen in liquid nitrogen. Blood was centrifuged for 3 min at 10000 g within 15 minutes of collection. Plasma was collected in low-binding microtubes (Nerbe plus) by adding 95 μL of plasma to 5 μL of 2% HCOOH to obtain 100 μL of plasma solution with HCOOH end concentration 0.1%. Plasma was frozen in liquid nitrogen and stored at −80° C.

Tissue samples were weighed in 2 mL micro centrifuge tubes. Microtubes were kept on ice throughout the procedure. Tissue homogenates were prepared using acidified (0.1% HCOOH) water by adding 4 times volume to weighed samples. Two metal beads were placed in each micro centrifuge tube and brain tissues homogenized using Bead Ruptor (Omni International, USA) for 60 s using speed 4 ms per cycle. Homogenates (96 uL) were transferred to 1.5 mL low binding micro centrifuge tubes. Homogenates were frozen and kept at −80° C. until sample extraction for LC/MS/MS analysis.

Extraction of spiked SD Rat EDTA plasma or brain homogenate standards was performed by adding to 100 μL of spiked rat plasma or brain homogenate standard sample of 400 μL of deproteinization solution, 1% formic acid in MeOH with IS (reserpine 15 ng/mL). Samples were vortex mixed for 10 sec at 2800 rpm and incubated on orbital shaker for 10 min at 200 rpm. Then vortex mixed for 10 sec at 2800 rpm and centrifugated at 12000 rpm for 30 min in 5° C. Supernatants were transferred to LC vials and used for analysis. Extraction of rat EDTA plasma or brain homogenate analytical samples was performed by adding to 96 μL of rat plasma or brain homogenate analytical sample of 4 μL of DMSO, vortex mix the samples. Then 400 μL of deproteinization solution, 1% formic acid in MeOH with IS (reserpine 15 ng/mL) were added. Samples were vortex mixed for 10 sec at 2800 rpm and incubated on orbital shaker for 10 min at 200 rpm. Then vortex mixed for 10 sec at 2800 rpm and centrifugated at 12000 rpm for 30 min in 5° C. Supernatants were transferred to LC vials and used for analysis.

Data collected for each of the various therapeutic agents tested (i.e. vatiquinone and Compounds 1-1, 2-1, 3-1, 4-1 and 5-1 are summarized in Table 1, below.

### Analysis of the Data

In summary, the data in Table 1 for the blood concentration analysis somewhat surprisingly suggests that as the molecular weight of the compound decreases, the gut absorption decreases. Often gut absorption is inversely proportional to size but in this case, as size increases, gut absorption does also. For example, vatiquinone and Compound 1-1 (MW 440.67) both exhibit a plasma concentration of about 500 ng/mL. The plasma concentration for Compounds 2-1 and 3-1 (MW 372.55) both drop to about 100 ng/mL (approximately a 5 fold drop). The plasma concentration for Compounds 4-1 and 5-1 (MW 304.43) drop significantly again (approximately a 20 fold drop). There does not appear to be any significant differentiation in gut uptake between R- and S-configurations of the same molecule.

In summary, the data in Table 1 for the brain concentration data suggests that Compounds 2-1 and 3-1 exhibit brain levels comparable to those observed with vatiquinone. This is surprising given the almost 5 times lower plasma concentration. Because the brain concentration is comparable to that observed for vatiquinone (despite the lower plasma concentration), this data suggests that Compounds 2-1 and 3-1 may more easily pass through the blood brain barrier (BBB). Given the very low concentrations of Compounds 4-1 and 5-1 in the blood, it is not surprising that there was no concentration of these compounds detected in the brain samples. Collectively the blood and brain concentration data suggest that both 2-1 and 3-1 (but particularly Compound 3-1) may be competitive with vatiquinone in treating neurological disease (e.g. Friedreich's ataxia) where ferroptosis is a contributing factor. Because the plasma concentration in Example 7 is dependent on gut uptake of the therapeutic agent being tested in the subjects, Compounds 2-1 and 3-1 may also be superior to vatiquinone if administered by another route that does not require gut uptake (e.g. subcutaneous or intravenous injection) because of their apparent superior ability to cross the BBB. Furthermore, because only gut absorption (i.e. oral administration) was examined in this experiment, the other compound tested (e.g. Compounds 1-1, 4-1 and 5-1) may also prove useful given the BSO assay results and LogD data provided—if administered by other means.

TABLE 1

| Com- | | | | | BSO Assay | Brain | Plasma |
|---|---|---|---|---|---|---|---|
| pound | Stereo | | | | Mean | level* | Cₘₐₓ |
| ID | Config | MW | Log D¹ | Structure | EC₅₀ (nM) | ng/g | ng/mL |
| N/A | R | 440.67 | 7.81 | 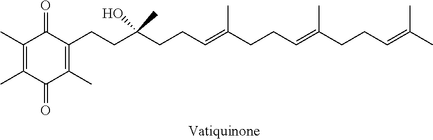 [SMILES: CC(C)=CCC/C(C)=C/CC/C(C)=C/CC[C@@](C)(O)CCC1=C(C)C(=O)C(C)=C(C)C1=O] 2-((R,6E,10E)-3-hydroxy-3,7,11,15-tetramethylhexadeca- 6,10,14-trien-1-yl)-3,5,6-trimethylcyclohexa-2,5-diene-1,4- dione | 40.7 +/− 18.7 (n = 18) | 20.8 | 457 |
| 1-1 | S | 440.67 | 7.81 | 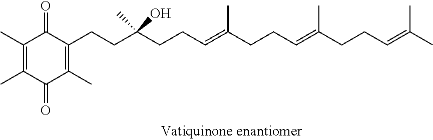 [SMILES: CC(C)=CCC/C(C)=C/CC/C(C)=C/CC[C@](C)(O)CCC1=C(C)C(=O)C(C)=C(C)C1=O] 2-((S,6E,10E)-3-hydroxy-3,7,11,15-tetramethylhexadeca- 6,10,14-trien-1-yl)-3,5,6-trimethylcyclohexa-2,5-diene-1,4- dione | 16.6 +/− 6.3 | 8.3 | 503 |
| 2-1 | R | 372.55 | 6.15 | 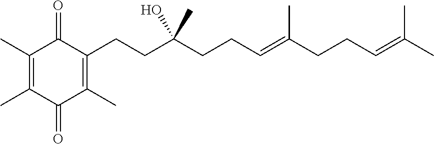 [SMILES: CC(C)=CCC/C(C)=C/CC[C@@](C)(O)CCC1=C(C)C(=O)C(C)=C(C)C1=O] (R,E)-2-(3-hydroxy-3,7,11-trimethyldodeca-6,10-dien- 1-yl)-3,5,6-trimethylcyclohexa-2,5-diene-1,4-dione | 140.9 +/− 32.9 (n = 3) | 24.5 | 121 |
| 3-1 | S | 372.55 | 6.15 | 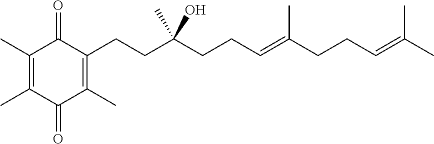 [SMILES: CC(C)=CCC/C(C)=C/CC[C@](C)(O)CCC1=C(C)C(=O)C(C)=C(C)C1=O] (S,E)-2-(3-hydroxy-3,7,11-trimethyldodeca-6,10-dien- 1-yl)-3,5,6-trimethylcyclohexa-2,5-diene-1,4-dione | 41.2 +/− 23.9 (n = 2) | 22.0 | 98 |
| 4-1 | R | 304.43 | 4.49 | 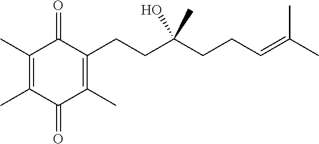 [SMILES: CC(C)=CCC[C@@](C)(O)CCC1=C(C)C(=O)C(C)=C(C)C1=O] (R)-2-(3-hydroxy-3,7-dimethyloct-6- en-1-yl)-3,5,6-trimethylcyclohexa- 2,5-diene-1,4-dione | 90.5 +/− 30.3 (n = 4) | 0 | 5.5 |
| 5-1 | S | 304.43 | 4.49 | 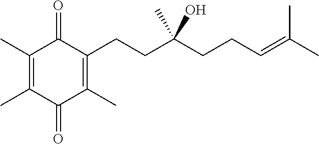 [SMILES: CC(C)=CCC[C@](C)(O)CCC1=C(C)C(=O)C(C)=C(C)C1=O] (S)-2-(3-hydroxy-3,7-dimethyloct-6- en-1-yl)-3,5,6-trimethylcyclohexa-2,5- diene-1,4-dione | 39.0 (n = 1) | 0 | 3.2 |

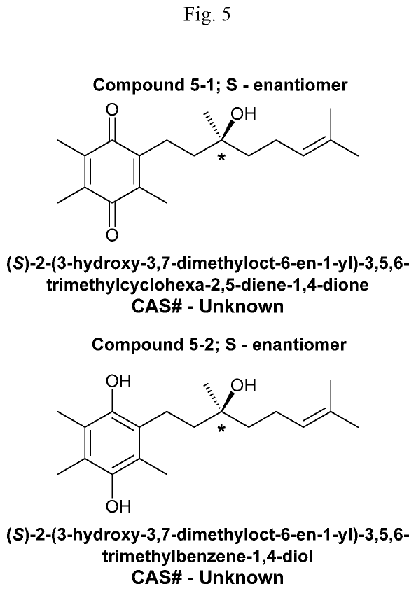

1. This column is included based on the teachings of Erb et al., PLoSONE, (2012), 7:4e36153 (Fig. 5) which suggest that the optimal Log D is between about 2-7.

### Example 8: Synthesis of 2-((S,6E,10E)-3-hydroxy-3,7,11,15-tetramethylhexadeca-6,10,14-trien-1-yl)-3,5,6-trimethylbenzene-1,4-diol (Compound 2; No known CAS#)—Prophetic Example—Follows the Procedure Described in WO2021/067836 (Example 8, Scheme 9) for Reduction of Vatiquinone to its Hydroquinone Analog.

This process was performed previously on vatiquinone and on various analogs of similar composition and found to be suitable in all cases. This procedure is a simple reduction and should be applicable to each of Compounds 1-1, 2-1, 3-1, 4-1 and 5.1. Generally the reduced compounds (i.e. the hydroquinone form—Compounds 1-2, 2-2, 3-2, 4-2 and 5-2) are easily oxidized back to their quinones in the presence of molecular oxygen so care should be taken during workup, purification and storage of the hydroquinone form to exclude the presence of oxygen.

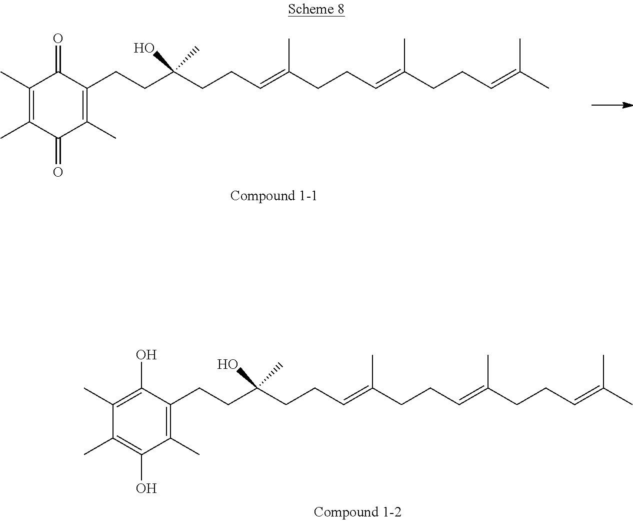

Compound 1-1 (about 45 μmol) is dissolved in mixture of MeOH/THF (1 mL+1 mL) under an argon atmosphere. Then NaBH₄ (3 mg) is added and the reaction mixture is stirred at r.t. for 1 hr. Next, the reaction mixture is quenched by the addition of Et₂O (30 mL) and aq. NH₄Cl (10 mL) with stirring at r.t. for 2 min. The aqueous phase is separated and the organic phase is washed with brine (10 mL), dried over anhydrous Na₂SO₄ and concentrated under reduced pressure. The crude product is purified by column chromatography to yield about 0.015 g (74% yield) of Compound 1-2.

Expected: ¹H-NMR (CDCl₃, 400 MHZ): δ=5.17-5.08 (m, 3H), 2.74-2.71 (m, 2H), 2.19-2.16 (m, 9H), 2.11-1.96 (m, 11H), 1.71-1.68 (m, 5H), 1.62-1.56 (m, 11H), 1.25 (s, 4H).

Expected: ¹³C-NMR (101 MHz, Chloroform-d) δ 145.9, 145.5, 136.1, 135.3, 131.4, 125.9, 124.5, 124.2, 124.0, 121.8, 120.7, 119.1, 74.0, 41.9, 41.0, 40.0, 30.5, 26.9, 26.7, 26.7, 25.8, 23.0, 20.7, 17.8, 16.2, 16.2, 12.5, 12.4, 12.2.

Expected: HRMS, [M+H]: C₂₉H₄₇O₃ (calculated: 443.3525). Found: 443.3516.

### EQUIVALENTS

The present application is not to be limited in terms of the particular embodiments described in this application, which are intended as single illustrations of individual aspects of the present technology. Many modifications and variations of the present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present application is to be limited only by the terms of the appended claims, along with the full scope of equivalents to which such claims are entitled. It is to be understood that the present application is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible sub-ranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for treating, preventing, inhibiting, ameliorating or delaying the onset of a disease, disorder or condition associated with ferroptosis in a mammalian subject suffering from a said disease, disorder or condition comprising administering to the subject a therapeutically effective amount of one or more of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione, 2-((S,6E,10E)-3-hydroxy-3,7,11,15-tetramethylhexadeca-6,10,14-trien-1-yl)-3,5,6-trimethylbenzene-1,4-diol, (R,E)-2-(3-hydroxy-3,7,11-trimethyldodeca-6,10-dien-1-yl)-3,5,6-trimethylcyclohexa-2,5-diene-1,4-dione, (R,E)-2-(3-hydroxy-3,7,11-trimethyldodeca-6,10-dien-1-yl)-3,5,6-trimethylbenzene-1,4-diol, (S,E)-2-(3-hydroxy-3,7,11-trimethyldodeca-6,10-dien-1-yl)-3,5,6-trimethylcyclohexa-2,5-diene-1,4-dione, (S,E)-2-(3-hydroxy-3,7,11-trimethyldodeca-6,10-dien-1-yl)-3,5,6-trimethylbenzene-1,4-diol, (R)-2-(3-hydroxy-3,7-dimethyloct-6-en-1-yl)-3,5,6-trimethylcyclohexa-2,5-diene-1,4-dione, (R)-2-(3-hydroxy-3,7-dimethyloct-6-en-1-yl)-3,5,6-trimethylbenzene-1,4-diol, (S)-2-(3-hydroxy-3,7-dimethyloct-6-en-1-yl)-3,5,6-trimethylcyclohexa-2,5-diene-1,4-dione, or (S)-2-(3-hydroxy-3,7-dimethyloct-6-en-1-yl)-3,5,6-trimethylbenzene-1,4-diol, or a pharmaceutically acceptable salt, hydrate, solvate, and/or tautomer of any of the foregoing.

2. The method of claim 1, wherein the disease, disorder or condition is Friedreich's ataxia.

3. The method of claim 1, wherein the disease, disorder or condition is Leigh syndrome.

4. The method of claim 1, wherein the disease, disorder or condition is Leber's Hereditary Optic Neuropathy (LHON).

5. The method of claim 1, wherein the disease, disorder or condition is (proliferative, non-proliferative, diabetic or hypertensive) retinopathy.

6. The method of claim 1, wherein the disease, disorder or condition is refractory epilepsy.

7. The method of claim 1, wherein the disease, disorder or condition is a neurological disease or disorder selected from Parkinson's disease (PD), Alzheimer's disease (AD), Huntington's disease (HS) and Amyotrophic Lateral Sclerosis (ALS).

8. The method of claim 1, wherein the disease, disorder or condition is ischemic stroke, or a cardiomyopathy selected from cardiac ischemia-reperfusion injury, myocardial infarction, Barth cardiomyopathy, hypertrophic cardiomyopathy and heart failure.

9. The method of claim 1, wherein the disease, disorder or condition is renal injury, renal ischemia reperfusion injury or acute renal failure.

10. The method of claim 1, wherein the effective amount of the one or more of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione, 2-((S,6E,10E)-3-hydroxy-3,7,11,15-tetramethylhexadeca-6,10,14-trien-1-yl)-3,5,6-trimethylbenzene-1,4-diol, (R,E)-2-(3-hydroxy-3,7,11-trimethyldodeca-6,10-dien-1-yl)-3,5,6-trimethylcyclohexa-2,5-diene-1,4-dione, (R,E)-2-(3-hydroxy-3,7,11-trimethyldodeca-6,10-dien-1-yl)-3,5,6-trimethylbenzene-1,4-diol, (S,E)-2-(3-hydroxy-3,7,11-trimethyldodeca-6,10-dien-1-yl)-3,5,6-trimethylcyclohexa-2,5-diene-1,4-dione, (S,E)-2-(3-hydroxy-3,7,11-trimethyldodeca-6,10-dien-1-yl)-3,5,6-trimethylbenzene-1,4-diol, (R)-2-(3-hydroxy-3,7-dimethyloct-6-en-1-yl)-3,5,6-trimethylcyclohexa-2,5-diene-1,4-dione, (R)-2-(3-hydroxy-3,7-dimethyloct-6-en-1-yl)-3,5,6-trimethylbenzene-1,4-diol, (S)-2-(3-hydroxy-3,7-dimethyloct-6-en-1-yl)-3,5,6-trimethylcyclohexa-2,5-diene-1,4-dione, or (S)-2-(3-hydroxy-3,7-dimethyloct-6-en-1-yl)-3,5,6-trimethylbenzene-1,4-diol, or a pharmaceutically acceptable salt, hydrate, solvate, and/or tautomer of any of the foregoing is administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

11. The method of claim 1, wherein the effective amount of the one or more of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione, 2-((S,6E,10E)-3-hydroxy-3,7,11,15-tetramethylhexadeca-6,10,14-trien-1-yl)-3,5,6-trimethylbenzene-1,4-diol, (R,E)-2-(3-hydroxy-3,7,11-trimethyldodeca-6,10-dien-1-yl)-3,5,6-trimethylcyclohexa-2,5-diene-1,4-dione, (R,E)-2-(3-hydroxy-3,7,11-trimethyldodeca-6,10-dien-1-yl)-3,5,6-trimethylbenzene-1,4-diol, (S,E)-2-(3-hydroxy-3,7,11-trimethyldodeca-6,10-dien-1-yl)-3,5,6-trimethylcyclohexa-2,5-diene-1,4-dione, (S,E)-2-(3-hydroxy-3,7,11-trimethyldodeca-6,10-dien-1-yl)-3,5,6-trimethylbenzene-1,4-diol, (R)-2-(3-hydroxy-3,7-dimethyloct-6-en-1-yl)-3,5,6-trimethylcyclohexa-2,5-diene-1,4-dione, (R)-2-(3-hydroxy-3,7-dimethyloct-6-en-1-yl)-3,5,6-trimethylbenzene-1,4-diol, (S)-2-(3-hydroxy-3,7-dimethyloct-6-en-1-yl)-3,5,6-trimethylcyclohexa-2,5-diene-1,4-dione, or (S)-2-(3-hydroxy-3,7-dimethyloct-6-en-1-yl)-3,5,6-trimethylbenzene-1,4-diol, or a pharmaceutically acceptable salt, hydrate, solvate, and/or tautomer of any of the foregoing is administered for 6 weeks or more, 12 weeks or more, 24 weeks or more, 48 weeks or more, 96 weeks or more, 1 year or more, 2 years or more, 5 years or more or 10 years or more.

12. The method of claim 1, wherein the mammalian subject is a human.

13. A method for treating, preventing, inhibiting, ameliorating or delaying the onset of Friedreich's ataxia in a mammalian subject, comprising administering to the subject a therapeutically effective amount of one or more of 2-[(3S,6E,10E)-3-hydroxy-3,7,11, 15-tetramethyl-6,10,14-hexadecatrien-1-yl]-3,5,6-trimethyl-2,5-cyclohexadiene-1,4-dione, 2-((S,6E,10E)-3-hydroxy-3,7,11,15-tetramethylhexadeca-6,10,14-trien-1-yl)-3,5,6-trimethylbenzene-1,4-diol, (R,E)-2-(3-hydroxy-3,7,11-trimethyldodeca-6,10-dien-1-yl)-3,5,6-trimethylcyclohexa-2,5-diene-1,4-dione, (R,E)-2-(3-hydroxy-3,7,11-trimethyldodeca-6,10-dien-1-yl)-3,5,6-trimethylbenzene-1,4-diol, (S,E)-2-(3-hydroxy-3,7,11-trimethyldodeca-6, 10-dien-1-yl)-3,5,6-trimethylcyclohexa-2,5-diene-1,4-dione, (S,E)-2-(3-hydroxy-3,7,11-trimethyldodeca-6,10-dien-1-yl)-3,5,6-trimethylbenzene-1,4-diol, (R)-2-(3-hydroxy-3,7-dimethyloct-6-en-1-yl)-3,5,6-trimethylcyclohexa-2,5-diene-1,4-dione, (R)-2-(3-hydroxy-3,7-dimethyloct-6-en-1-yl)-3,5,6-trimethylbenzene-1,4-diol, (S)-2-(3-hydroxy-3,7-dimethyloct-6-en-1-yl)-3,5,6-trimethylcyclohexa-2,5-diene-1,4-dione, or (S)-2-(3-hydroxy-3,7-dimethyloct-6-en-1-yl)-3,5,6-trimethylbenzene-1,4-diol, or a pharmaceutically acceptable salt, hydrate, solvate, and/or tautomer of any of the foregoing.

14. The method of claim 13, wherein the mammalian subject is human.

15. The method of claim 13, wherein the effective amount of the one or more of 2-[(3S,6E,10E)-3-hydroxy-3,7,11,15-tetramethyl-6,10,14-hexadecatrien-1-yl]-3,5,6-trimethyl-2,5-cyclohexadiene-1,4-dione, 2-((S,6E,10E)-3-hydroxy-3,7,11,15-tetramethylhexadeca-6,10,14-trien-1-yl)-3,5,6-trimethylbenzene-1,4-iol, (R,E)-2-(3-hydroxy-3,7,11-trimethyldodeca-6,10-dien-1-yl)-3,5,6-trimethylcyclohexa-2,5-diene-1,4-dione, (R,E)-2-(3-hydroxy-3,7,11-trimethyldodeca-6,10-dien-1-yl)-3,5,6-trimethylbenzene-1,4-diol, (S,E)-2-(3-hydroxy-3,7,11-trimethyldodeca-6,10-dien-1-yl)-3,5,6-trimethylcyclohexa-2,5-diene-1,4-dione, (S,E)-2-(3-hydroxy-3,7,11-trimethyldodeca-6,10-dien-1-yl)-3,5,6-trimethylbenzene-1,4-diol, (R)-2-(3-hydroxy-3,7-dimethyloct-6-en-1-yl)-3,5,6-trimethylcyclohexa-2,5-diene-1,4-dione, (R)-2-(3-hydroxy-3,7-dimethyloct-6-en-1-yl)-3,5,6-trimethylbenzene-1,4-diol, (S)-2-(3-hydroxy-3,7-dimethyloct-6-en-1-yl)-3,5,6-trimethylcyclohexa-2,5-diene-1,4-dione, or (S)-2-(3-hydroxy-3,7-dimethyloct-6-en-1-yl)-3,5,6-trimethylbenzene-1,4-diol, or a pharmaceutically acceptable salt, hydrate, solvate, and/or tautomer of any of the foregoing is administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

16. The method of claim 13, wherein the effective amount of the one or more of 2-[(3S,6E,10E)-3-hydroxy-3,7,11,15-tetramethyl-6,10, 14-hexadecatrien-1-yl]-3,5,6-trimethyl-2,5-cyclohexadiene-1,4-dione, 2-((S,6E,10E)-3-hydroxy-3,7,11,15-tetramethylhexadeca-6,10,14-trien-1-yl)-3,5,6-trimethylbenzene-1,4-diol, (R,E)-2-(3-hydroxy-3,7,11-trimethyldodeca-6,10-dien-1-yl)-3,5,6-trimethylcyclohexa-2,5-diene-1,4-dione, (R,E)-2-(3-hydroxy-3,7,11-trimethyldodeca-6,10-dien-1-yl)-3,5,6-trimethylbenzene-1,4-diol, (S,E)-2-(3-hydroxy-3,7,11-trimethyldodeca-6,10-dien-1-yl)-3,5,6-trimethylcyclohexa-2,5-diene-1,4-dione, (S,E)-2-(3-hydroxy-3,7,11-trimethyldodeca-6,10-dien-1-yl)-3,5,6-trimethylbenzene-1,4-diol, (R)-2-(3-hydroxy-3,7-dimethyloct-6-en-1-yl)-3,5,6-trimethylcyclohexa-2,5-diene-1,4-dione, (R)-2-(3-hydroxy-3,7-dimethyloct-6-en-1-yl)-3,5,6-trimethylbenzene-1,4-diol, (S)-2-(3-hydroxy-3,7-dimethyloct-6-en-1-yl)-3,5,6-trimethylcyclohexa-2,5-diene-1,4-dione, or (S)-2-(3-hydroxy-3,7-dimethyloct-6-en-1-yl)-3,5,6-trimethylbenzene-1,4-diol, or a pharmaceutically acceptable salt, hydrate, solvate, and/or tautomer of any of the foregoing is administered for 6 weeks or more, 12 weeks or more, 24 weeks or more, 48 weeks or more, 96 weeks or more, 1 year or more, 2 years or more, 5 years or more or 10 years or more.

17. (canceled)

18. A pharmaceutical formulation or medicament for treating, preventing, inhibiting, ameliorating or delaying the onset of a disease, disorder or condition associated with ferroptosis in a mammalian subject, said pharmaceutical formulation or medicament comprising an effective amount of one or more of 2-[(3S,6E, 10E)-3-hydroxy-3,7, 11,15-tetramethyl-6,10,14-hexadecatrien-1-yl]-3,5,6-trimethyl-2,5-cyclohexadiene-1,4-dione, 2-((S,6E,10E)-3-hydroxy-3,7,11,15-tetramethylhexadeca-6,10,14-trien-1-yl)-3,5,6-trimethylbenzene-1,4-diol, (R,E)-2-(3-hydroxy-3,7,11-trimethyldodeca-6,10-dien-1-yl)-3,5,6-trimethylcyclohexa-2,5-diene-1,4-dione, (R,E)-2-(3-hydroxy-3,7,11-trimethyldodeca-6,10-dien-1-yl)-3,5,6-trimethylbenzene-1,4-diol, (S,E)-2-(3-hydroxy-3,7,11-trimethyldodeca-6,10-dien-1-yl)-3,5,6-trimethylcyclohexa-2,5-diene-1,4-dione, (S,E)-2-(3-hydroxy-3,7,11-trimethyldodeca-6,10-dien-1-yl)-3,5,6-trimethylbenzene-1,4-diol, (R)-2-(3-hydroxy-3,7-dimethyloct-6-en-1-yl)-3,5,6-trimethylcyclohexa-2,5-diene-1,4-dione, (R)-2-(3-hydroxy-3,7-dimethyloct-6-en-1-yl)-3,5,6-trimethylbenzene-1,4-diol, (S)-2-(3-hydroxy-3,7-dimethyloct-6-en-1-yl)-3,5,6-trimethylcyclohexa-2,5-diene-1,4-dione, or (S)-2-(3-hydroxy-3,7-dimethyloct-6-en-1-yl)-3,5,6-trimethylbenzene-1,4-diol, or a pharmaceutically acceptable salt, hydrate, solvate, and/or tautomer of any of the foregoing.

19. The pharmaceutical formulation or medicament of claim 18, wherein the disease, disorder or condition is Friedreich's ataxia.

20. The pharmaceutical formulation or medicament of claim 18, wherein the disease, disorder or condition is Leigh syndrome.

21. The pharmaceutical formulation or medicament of claim 18, wherein the disease, disorder or condition is Leber's Hereditary Optic Neuropathy (LHON).

22. The pharmaceutical formulation or medicament of claim 18, wherein the disease, disorder or condition is (proliferative, non-proliferative, diabetic or hypertensive) retinopathy.

23. The pharmaceutical formulation or medicament of claim 18, wherein the disease, disorder or condition is refractory epilepsy.

24. The pharmaceutical formulation or medicament of claim 18, wherein the disease or disorder is a neurological disease, disorder or condition selected from Parkinson's disease (PD), Alzheimer's disease (AD), Huntington's disease (HS) and Amyotrophic Lateral Sclerosis (ALS).

25. The pharmaceutical formulation or medicament of claim 18, wherein the disease, disorder or condition is ischemic stroke, or a cardiomyopathy selected from cardiac ischemia-reperfusion injury, myocardial infarction, Barth cardiomyopathy, hypertrophic cardiomyopathy and heart failure.

26. The pharmaceutical formulation or medicament of claim 18, wherein the disease, disorder or condition is renal injury, renal ischemia reperfusion injury or acute renal failure.

27. The pharmaceutical formulation or medicament of claim 18, wherein the pharmaceutical formulation or medicament is administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

28. The pharmaceutical formulation or medicament of claim 18, wherein the pharmaceutical formulation or medicament is administered for 6 weeks or more, 12 weeks or more, 24 weeks or more, 48 weeks or more, 96 weeks or more, 1 year or more, 2 years or more, 5 years or more or 10 years or more.

29. The pharmaceutical formulation or medicament of claim 18, wherein the mammalian subject is a human.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A compound of Formula I, Formula II, Formula III, Formula IV, Formula V, Formula VI, Formula VII, or Formula VIII: 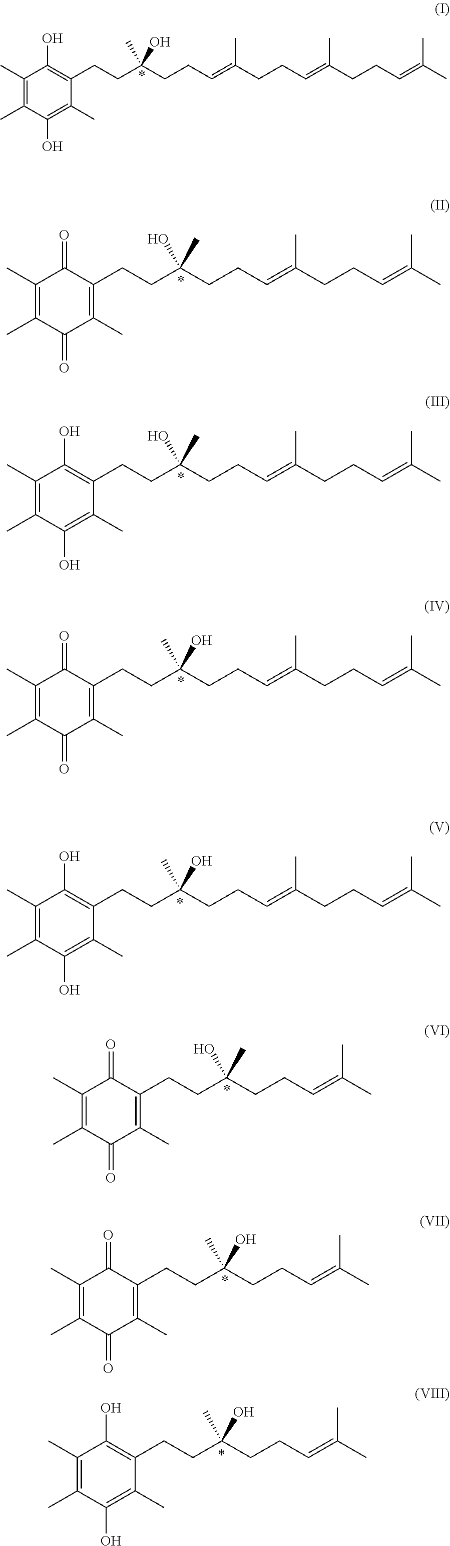
or a pharmaceutically acceptable salt, hydrate, solvate, and/or tautomer thereof of any of the foregoing, wherein the compound optionally comprises at least one deuterium atom substituted for a hydrogen atom.

47. (canceled)

48. (canceled)

49. (canceled)

50. (canceled)

51. (canceled)

52. (canceled)

53. (canceled)

54. The compound of claim 46, wherein the compound is at least 80% enantiomerically pure.

55.-70. (Canceled)
